(19) United States
(12) Patent Application Publication
(10) Pub. No.: US 2026/0027104 A1
(43) Pub. Date: Jan. 29, 2026

(54) NOVEL NAPHTHYL AND ISOQUINOLINE SULFONAMIDE DERIVATIVES

(71) Applicant: Hoffmann-La Roche Inc., Little Falls, NJ (US)

(72) Inventors: Guido GALLEY, Rheinfelden (DE); Luca Claudio GOBBI, Buus (CH); Wolfgang GUBA, Muellheim (DE); Dmitry MAZUNIN, Grenzach-Wyhlen (DE); Emmanuel PINARD, Linsdorf (FR); Antonio RICCI, Biel-Benken (CH)

(73) Assignee: Hoffmann-La Roche Inc., Little Falls, NJ (US)

(21) Appl. No.: 18/993,966

(22) PCT Filed: Jul. 18, 2023
(86) PCT No.: PCT/EP2023/069850

§ 371 Date: Jan. 13, 2025

(30) Foreign Application Priority Data
Jul. 20, 2022 (EP) 22185897.0
Aug. 2, 2022 (EP) 22188190.7

(51) Int. Cl. A61K 31/4709; A61K 31/4375; A61K 31/44; A61K 31/4433; A61K 31/47; A61K 31/472; A61K 31/4725; A61K 31/506; A61K 47/02; A61K 47/14; A61K 47/36; A61K 47/38; C07C 303/38; C07C 311/21; C07D 213/76; C07D 215/36; C07D 401/12; C07D 405/12; C07D 405/14; C07D 471/04

(52) U.S. Cl. CPC A61K 31/4709; A61K 31/4375; A61K 31/44; A61K 31/4433; A61K 31/47; A61K 31/472; A61K 31/4725; A61K 31/506; A61K 47/02; A61K 47/14; A61K 47/36; A61K 47/38; C07C 303/38; C07C 311/21; C07D 213/76; C07D 215/36; C07D 401/12; C07D 405/12; C07D 405/14; C07D 471/04

(57) ABSTRACT

The invention relates to novel compounds having the general formula (I) wherein R¹, R², X¹, X², X³ and W are as described herein, composition including the compounds and methods of using the compounds.

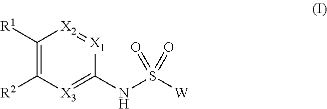

The present invention relates to organic compounds useful for therapy and/or prophylaxis in a mammal, and in particular to compounds that modulate GPR17 activity.

The present invention provides novel compounds of formula I

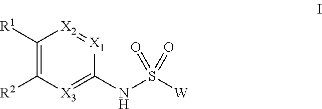

wherein,
R¹ is selected from cyanoalkyl, haloalkoxy, haloalkyl, cyclopropyl and oxetanyl;
R² is selected from alkoxy, H or halo;
X₁ is N and X₂ is CR⁴ and X₃ is N, or
X₁ is CR³, X₂ is CR⁴, and X₃ is N or CR⁵, or
X₁ is CR³, X₂ is N, and X₃ is CR⁵;
R³ is selected from alkoxy, H, halo, haloalkoxy
R⁴ is selected from alkoxy, H, halo
R⁵ is selected from H, halo
W is selected from Ring A, Ring B, or Ring C

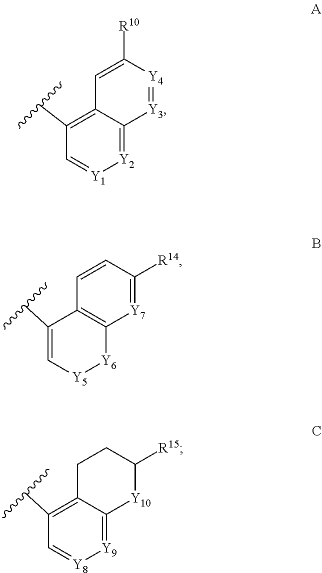

Y₁ is CR⁶, Y₂ is CR⁷, Y₃ is CR⁸ and Y₄ is CR⁹, or
Y₁ is N, Y₂ is CR⁷, Y₃ is CR⁸ and Y₄ is CR⁹, or
Y₁ is N, Y₂ is CR⁷, Y₃ is N and Y₄ is CR⁹, or
Y₁ is CR⁶, Y₂ is N, Y₃ is CR⁸ and Y₄ is CR⁹, or
Y₁ is CR⁶, Y₂ is CR⁷, Y₃ is N and Y₄ is CR⁹, or
Y₁ is CR⁶, Y₂ is CR⁷, Y₃ is CR⁸ and Y₄ is N;
R⁶ is H or alkylamino;
R⁷ is alkoxy, dimethylamino, H, methyl, methylamino, OH;
R⁸ is cyano, dimethylamino, H, halo;
R⁹ is alkoxy, cyclopropyl, haloalkyl dimethylamino, H, halo, alkyl;
R¹⁰ is H or halo;
Y₅ is NR¹¹, Y₆ is —C(═O)—, and Y₇ is CR¹³ or N, or
Y₅ is —C(═O)—, Y₆ is NR¹² and Y₇ is CR¹³;
R¹¹ is alkyl or H;
R¹² is alkyl or H;
R¹³ is H, alkyl or halo;
R¹⁴ is H, alkyl, halo, or haloalkyl;
Y₈ is —CH— or —N—;
Y₉ is —CH— or —N—;
Y₁₀ is —CH₂— or —O—;
R¹⁵ is H, alkyl, or haloalkyl;
and pharmaceutically acceptable salts thereof.

Furthermore, the invention includes all racemic mixtures, all their corresponding enantiomers and/or optical isomers.

### BACKGROUND OF THE INVENTION

Myelination is a process that occurs robustly during development and despite the abundant presence of oligodendrocyte precursor cells (OPCs) throughout the adult CNS, the transition to myelinating oligodendrocytes and the production of restorative myelin sheaths around denuded axons is impaired in chronic demyelinating diseases. During development, myelination proceeds in a very orderly manner, with OPCs, characterized by expression of markers such as neural/glial antigen 2 (NG2) and platelet-derived growth factor alpha (PDGFRα), differentiating into oligodendrocytes which lose NG2 and PDGFRα expression and gain the expression of markers such as myelin basic protein (MBP) and myelin oligodendrocyte glycoprotein (MOG). The production of myelin by oligodendrocytes is a very tightly regulated process and in the CNS, this can be controlled by interactions with axons, well-understood in the peripheral but not in the central nervous system (Macklin, W. B. (2010). Sci. Signal. 3, pe32-pe32, “The myelin brake: When Enough Is Enough”). Myelination can also be controlled by internal brakes within oligodendrocytes themselves, through the transcription factor EB (TFEB)-PUMA axis or through GPR17 antagonism (Chen, Y., et al. (2009). Nat Neurosci 12, 1398-1406, “The oligodendrocyte-specific G protein-coupled receptor GPR17 is a cell-intrinsic timer of myelination”) (Sun, L. O., et al. (2018). Cell 175, 1811-1826.e21, “Spatiotemporal Control of CNS Myelination by Oligodendrocyte Programmed Cell Death through the TFEB-PUMA Axis”). Myelin serves not only to protect axons and facilitate neuronal transmission, but oligodendrocytes have also been shown to play an important role in metabolism of axons as well as in maintaining the electrolyte balance around axons (Schirmer, L., et al. (2014). Ann Neurol 75, 810-828, “Differential loss of KIR4.1 immunoreactivity in multiple sclerosis lesions”) (Simons, M., and Nave, K.-A. (2015). Cold Spring Harb Perspect Biol. 22, “Oligodendrocytes: Myelination and Axonal Support”).

GPR17 is a Class A orphan G protein-coupled receptor (GPCR). GPCRs are 7 domain transmembrane proteins that couple extracellular ligands with intracellular signaling via their intracellular association with small, heterotrimeric G-protein complexes consisting of G_{α}, G_{β}, G_{γ} subunits. It is the coupling of the GPCR to the Ga subunit that confers results in downstream intracellular signaling pathways. GPR17 is known to be coupled directly to G_{αi/o}, which leads to inhibition of adenylate cyclase activity, resulting in a reduction in cyclic AMP production (cAMP). GPR17 has also been shown to couple to G_{q/11}, that targets phospholipase C. Activation of phospholipase C leads to the cleavage of phosphatidylinositol 4,5-bisphosphate which produces inositol triphosphate (IP₃) and diacylglycerol (DAG). IP₃ consequently binds to the IP₃ receptor on the endoplasmic reticulum and causes an increase in intracellular calcium levels (Hanlon, C. D., and Andrew, D. J. (2015). J Cell Sci. 128, 3533-3542, “Outside-in signaling—a brief review of GPCR signaling with a focus on the Drosophila GPCR family”) (Inoue, A., et al. (2019), Cell 177, 1933-1947.e25, “Illuminating G-Protein-Coupling Selectivity of GPCRs”).

The role of GPR17 in myelination was first identified in a screen of the optic nerves of Olig1 knockout mice to identify genes regulating myelination. GPR17 expression was found to be expressed only in the myelinating cells of the CNS and absent from the Schwann cells, the peripheral nervous system's myelinating cells. The expression of GPR17 was found to be exclusively expressed in the oligodendrocyte lineage cells and was downregulated in myelinating oligodendrocyte (Chen, Y., et al. (2009)). Specifically, GPR17 expression is found to be present at low levels early on in the OPC and increases in the pre-myelinating oligodendrocyte before the expression is downregulated in the mature, myelinating oligodendrocyte (Boda, E., et al. (2011), Glia 59, 1958-1973, “The GPR17 receptor in NG2 expressing cells: Focus on in vivocell maturation and participation in acute trauma and chronic damage”) (Dziedzic, A., et al. (2020). Int. J. Mol. Sci. 21, 1852, “The gpr17 receptor—a promising goal for therapy and a potential marker of the neurodegenerative process in multiple sclerosis”) (Fumagalli, M. et al. (2011), J Biol Chem 286, 10593-10604, “Phenotypic changes, signaling pathway, and functional correlates of GPR17-expressing neural precursor cells during oligodendrocyte differentiation”). GPR17 knockout animals were shown to exhibit precocious myelination throughout the CNS and conversely, transgenic mice overexpressing GPR17 in oligodendrocytes with the CNP-Cre (2′, 3′-cyclic-nucleotide 3′-phosphodiesterase) promoter exhibited myelinogenesis defects, in line with what is to be expected of a cell-intrinsic brake on the myelination process (Chen, Y., et al. (2009)). Furthermore, loss of GPR17 enhances remyelination following demyelination with lysophosphatidylcholine-induced demyelination (Lu, C., Dong, et al. (2018), Sci. Rep. 8, 4502, “G-Protein-Coupled Receptor Gpr17 Regulates Oligodendrocyte Differentiation in Response to Lysolecithin-Induced Demyelination”). As such, antagonism of GPR17 that promotes the differentiation of oligodendrocyte lineage cells into mature, myelinating oligodendrocytes would lead to increase in myelination following demyelination.

Multiple sclerosis (MS) is a chronic neurodegenerative disease that is characterized by the loss of myelin, the protective fatty lipid layer surrounding axons, in the central nervous system (CNS). Prevention of myelin loss or remyelination of denuded axons is thought to prevent axonal degeneration and thus prevent progression of the disease (Franklin, R. J. (2002), Nat Rev Neurosci 3, 705-714, “Why does remyelination fail in multiple sclerosis?”). Due to the restorative impact that myelin repair has on the central nervous system, such a treatment will benefit all types of MS namely relapse-remitting, secondary progressive, primary progressive and progressive relapsing MS. Reparation of lost myelin will alleviate neurological symptoms associated with MS due to the neuroprotective effect of preserving axons.

Due to the essential role that myelination plays in functioning of the nervous system, facilitating OPC to oligodendrocyte differentiation has the potential to impact multiple diseases where white matter defects/irregularities due to either loss of myelinating oligodendrocytes or hampered differentiation of OPCs to oligodendrocytes have been observed, due to the disease itself or inflammation. This is in addition to the diseases where GPR17 expression itself is altered.

The diseases that GPR17 antagonism can be thus used to yield a positive disease outcome include, but are not limited to:

### Direct Damage to Myelin Sheaths:

Metabolic conditions that lead to destruction of central myelin such as central pontine myelinolysis, extra-pontine myelinolysis due to overly-rapid correction of hyponatremia in conditions for instance, but not limited to, alcoholism, liver disease, immunosuppression after transplantation
Carbon monoxide poisoning where oligodendrocyte dysfunction and failure to regenerate has been reported in the deep white matter layers of the brain
Nutritional deficiency that results in myelin loss or failure to properly generate myelin during development
Virus-induced demyelination

### Primary Demyelinating Disorders

Multiple Sclerosis (relapse-remitting, secondary progressive, primary progressive and progressive relapsing MS)
Acute and multiphasic disseminated encephalomyelitis
Neuromyelitis optica spectrum disorders including optic neuritis
Transverse myelitis
Leukodystrophies such as adrenoleukodystrophy, adrenomyeloneuropathy and other inherited leukodystrophies that result in myelin loss
   CNS Disorders with Associated Myelin Loss:
Alzheimer's Disease
Schizophrenia
Parkinson's Disease
Huntington's disease
Amyotrophic lateral
Ischemia due to stroke

### Other Diseases:

Inflammation in the CNS for instance following encephalitis, primary angiitis, meningitis

The compounds of formula I bind to and modulates GPR17 activity.

The compounds of formula I are therefore particularly useful in the treatment of diseases related to GPR17 antagonism.

The compounds of formula I are particularly useful in the treatment or prophylaxis of multiple sclerosis (MS), conditions related to direct damage to myelin sheaths such as carbon monoxide poisoning or virus induced demyelination, primary demyelinating disorders such as acute and multiphasic disseminated encephalomyelitis, and other CNS disorders associated with myelin loss such as Alzheimer's disease, schizophrenia, Parkinson's disease and Huntington's disease.

### SUMMARY OF THE INVENTION

The present invention provides novel compounds of formula I

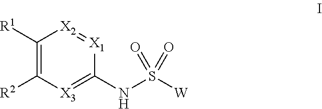

wherein,
R¹ is selected from cyanoalkyl, haloalkoxy, haloalkyl, cyclopropyl and oxetanyl;
R² is selected from alkoxy, H or halo;
X₁ is N and X₂ is CR⁴ and X₃ is N, or
X₁ is CR³, X₂ is CR⁴, and X₃ is N or CR⁵, or
X₁ is CR³, X₂ is N, and X₃ is CR⁵;
R³ is selected from alkoxy, H, halo, haloalkoxy;
R⁴ is selected from alkoxy, H, halo;
R⁵ is selected from H, halo;
W is selected from Ring A, Ring B, or Ring C;

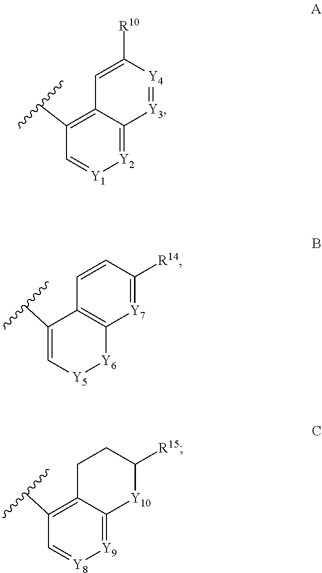

Y₁ is CR⁶, Y₂ is CR⁷, Y₃ is CR⁸ and Y₄ is CR⁹, or
Y₁ is N, Y₂ is CR⁷, Y₃ is CR⁸ and Y₄ is CR⁹, or
Y₁ is N, Y₂ is CR⁷, Y₃ is N and Y₄ is CR⁹, or
Y₁ is CR⁶, Y₂ is N, Y₃ is CR⁸ and Y₄ is CR⁹, or
Y₁ is CR⁶, Y₂ is CR⁷, Y₃ is N and Y₄ is CR⁹, or
Y₁ is CR⁶, Y₂ is CR⁷, Y₃ is CR⁸ and Y₄ is N;
R⁶ is H or alkylamino;
R⁷ is alkoxy, dimethylamino, H, methyl, methylamino, OH;
R⁸ is cyano, dimethylamino, H, halo;
R⁹ is alkoxy, cyclopropyl, haloalkyl dimethylamino, H, halo, alkyl;
R¹⁰ is H or halo;
Y₅ is NR¹¹, Y₆ is —C(═O)—, and Y₇ is CR¹³ or N, or
Y₅ is —C(═O)—, Y₆ is NR¹² and Y₇ is CR¹³;
R¹¹ is alkyl or H;
R¹² is alkyl or H;
R¹³ is H, alkyl or halo;
R¹⁴ is H, alkyl, halo, or haloalkyl;
Y₈ is —CH— or —N—;
Y₉ is —CH— or —N—;
Y₁₀ is —CH₂— or —O—;
R¹⁵ is H, alkyl, or haloalkyl;
and pharmaceutically acceptable salts thereof.

The term “alkyl” denotes a monovalent linear or branched saturated hydrocarbon group of 1 to 6 carbon atoms. In some embodiments, if not otherwise described, alkyl comprises 1 to 6 carbon atoms (C₁₋₆-alkyl), or 1 to 4 carbon atoms (C₁₋₄-alkyl). Examples of C₁₋₆-alkyl include methyl, ethyl, propyl, isopropyl, n-butyl, iso-butyl, sec-butyl, tert-butyl and pentyl. Particular alkyl groups include methyl and ethyl. When an alkyl residue having a specific number of carbons is named, all geometric isomers having that number of carbons may be encompassed. Thus, for example, “butyl” can include n-butyl, sec-butyl, isobutyl and t-butyl, and “propyl” can include n-propyl and isopropyl.

The term “alkoxy” denotes a group of the formula —O—R′, wherein R′ is a C₁₋₆-alkyl group. Examples of C₁₋₆-alkoxy groups include methoxy, ethoxy, n-propoxy, isopropoxy, n-butoxy, isobutoxy and tert-butoxy. Particular examples are methoxy.

The term “cyano” denotes a C—N group.

“Cyanoalkyl” means a moiety of the formula —R′—R″, where R′ is alkyl as defined herein and R″ is cyano or nitrile. Particular example is cyanomethyl.

The term “halogen”, “halide” and “halo” are used interchangeably herein and denote fluoro, chloro, bromo or iodo. Particular halogens are chloro and bromo.

The term “haloalkoxy” denotes a C₁₋₆-alkoxy group wherein at least one of the hydrogen atoms of the C₁₋₆-alkoxy group has been replaced by the same or different halogen atoms. Particular examples are difluoroethoxy and difluoromethoxy.

The term “haloalkyl” denotes a C₁₋₆-alkyl group wherein at least one of the hydrogen atoms of the C₁₋₆-alkyl group has been replaced by the same or different halogen atoms. Particular example is difluoroethyl.

The term “amino” denotes a —NH₂ group.

The term “alkylamino” denotes an amino group wherein one of the hydrogen atoms of the amino group has been replaced by an alkyl group. Example is methylamino.

The term “pharmaceutically acceptable salts” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid, particularly hydrochloric acid, and organic acids such as formic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein. In addition these salts may be prepared from addition of an inorganic base or an organic base to the free acid. Salts derived from an inorganic base include, but are not limited to, the sodium, potassium, lithium, ammonium, calcium, magnesium salts. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyamine resins. The compound of formula I can also be present in the form of zwitterions. Particularly preferred pharmaceutically acceptable salts of compounds of formula I are the salts formed with formic acid and the salts formed with hydrochloric acid yielding a hydrochloride, dihydrochloride or trihydrochloride salt.

The abbreviation uM means microMolar and is equivalent to the symbol μM.

The abbreviation uL means microliter and is equivalent to the symbol μL.

The abbreviation ug means microgram and is equivalent to the symbol μg.

The compounds of formula I can contain several asymmetric centers and can be present in the form of optically pure enantiomers, mixtures of enantiomers such as, for example, racemates, optically pure diastereoisomers, mixtures of diastereoisomers, diastereoisomeric racemates or mixtures of diastereoisomeric racemates.

According to the Cahn-Ingold-Prelog Convention the asymmetric carbon atom can be of the “R” or “S” configuration.

Also an embodiment of the present invention provides compounds according to formula I as described herein and pharmaceutically acceptable salts or esters thereof, in particular compounds according to formula I as described herein and pharmaceutically acceptable salts thereof, more particularly compounds according to formula I as described herein.

An embodiment of the present invention provides compounds according to formula I as described herein, wherein R¹ is cyanoalkyl, haloalkoxy, or cyclopropyl.

An embodiment of the present invention provides compounds according to formula I as described herein, wherein R² is alkoxy or halo.

An embodiment of the present invention provides compounds according to formula I as described herein, wherein R³ is alkoxy, halo, or haloalkoxy.

An embodiment of the present invention provides compounds according to formula I as described herein, wherein X₁ is CR³, X₂ is CR⁴, and X₃ is N or CR⁵, or X₁ is CR3, X₂ is N, and X₃ is CR⁵.

An embodiment of the present invention provides compounds according to formula I as described herein, wherein R⁴ is H.

An embodiment of the present invention provides compounds according to formula I as described herein, wherein R⁵ is H.

An embodiment of the present invention provides compounds according to formula I as described herein, wherein
Y₁ is CR⁶, Y₂ is CR⁷, Y₃ is CR⁸ and Y₄ is CR⁹, or
Y₁ is N, Y₂ is CR⁷, Y₃ is CR⁸ and Y₄ is CR⁹, or
Y₁ is N, Y₂ is CR⁷, Y₃ is N and Y₄ is CR⁹, or
Y₁ is CR⁶, Y₂ is CR⁷, Y₃ is N and Y₄ is CR⁹.

An embodiment of the present invention provides compounds according to formula I as described herein, wherein R⁶ is H.

An embodiment of the present invention provides compounds according to formula I as described herein, wherein R⁷ is H or methylamino.

An embodiment of the present invention provides compounds according to formula I as described herein, wherein R⁸ is dimethylamino, H or halo.

An embodiment of the present invention provides compounds according to formula I as described herein, wherein R⁹ is cyclopropyl, H, halo or alkyl.

An embodiment of the present invention provides compounds according to formula I as described herein, wherein R¹⁰ is H.

An embodiment of the present invention provides compounds according to formula I as described herein, wherein R¹¹ is H.

An embodiment of the present invention provides compounds according to formula I as described herein, wherein R¹² is H.

An embodiment of the present invention provides compounds according to formula I as described herein, wherein R¹³ is H or halo.

An embodiment of the present invention provides compounds according to formula I as described herein, wherein R¹⁴ is H or halo.

An embodiment of the present invention provides compounds according to formula I as described herein, wherein R¹⁴ is halo.

An embodiment of the present invention provides compounds according to formula I as described herein, wherein Y₈ is —CH—, Y₉ is —CH— and Y₁₀ is —CH— or —O—.

An embodiment of the present invention provides compounds according to formula I as described herein, wherein Y₈ is —CH—, Y₉ is —CH— and Y₁₀ is —O—.

An embodiment of the present invention provides compounds according to formula I as described herein, wherein R¹⁵ is H or alkyl.

An embodiment of the present invention provides compounds according to formula I as described herein, wherein R¹⁵ is alkyl.

An embodiment of the present invention provides compounds according to formula I as described herein, wherein
when R² is H, then X₂ is CR⁴ and R⁴ is not H;
when R¹ is haloalkyl, then R² is alkoxy; and
when R¹ is haloalkoxy and R² is halo, then X₂ is N.

An embodiment of the present invention provides compounds according to formula I as described herein, wherein
R¹ is selected from cyanoalkyl, haloalkoxy, haloalkyl, cyclopropyl and oxetanyl;
R² is selected from alkoxy, H or halo;
X₁ is N and X₂ is CR⁴ and X₃ is N, or
X₁ is CR³, X₂ is CR⁴, and X₃ is N or CR⁵, or
X₁ is CR³, X₂ is N, and X₃ is CR⁵;
R³ is selected from alkoxy, H, halo, haloalkoxy;
R⁴ is selected from alkoxy, H, halo;
R⁵ is selected from H, halo;
W is selected from Ring A, Ring B, or Ring C

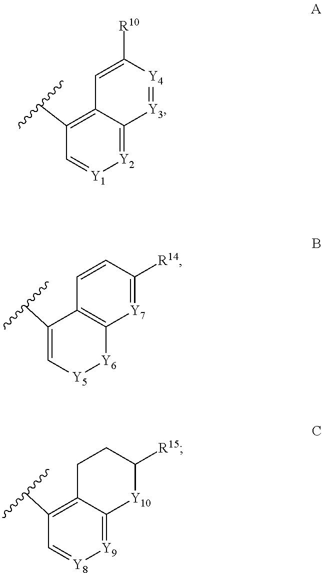

Y₁ is CR⁶, Y₂ is CR⁷, Y₃ is CR⁸ and Y₄ is CR⁹, or
Y₁ is N, Y₂ is CR⁷, Y₃ is CR⁸ and Y₄ is CR⁹, or
Y₁ is N, Y₂ is CR⁷, Y₃ is N and Y₄ is CR⁹, or
Y₁ is CR⁶, Y₂ is N, Y₃ is CR^{N} and Y₄ is CR⁹, or
Y₁ is CR⁶, Y₂ is CR⁷, Y₃ is Nand Y₄ is CR⁹, or
Y₁ is CR⁶, Y₂ is CR⁷, Y₃ is CR⁸ and Y₄ is N;
R⁶ is H or alkylamino;
R⁷ is alkoxy, dimethylamino, H, methyl, methylamino, OH;
R⁸ is cyano, dimethylamino, H, halo;
R⁹ is alkoxy, cyclopropyl, haloalkyl dimethylamino, H, halo, alkyl;
R¹⁰ is H or halo;
Y₅ is NR¹¹, Y₆ is —C(═O)—, and Y₇ is CR¹³ or N, or
Y₅ is —C(═O)—, Y₆ is NR¹² and Y₇ is CR¹³;
R¹¹ is alkyl or H;
R¹² is alkyl or H;
R¹³ is H, or halo:
R¹⁴ is H or halo:
Y₈ is —CH—;
Y₉ is —CH—;
Y₁₀ is —CH₂— or —O—;
R¹⁵ is H, or alkyl;
and pharmaceutically acceptable salts thereof.

An embodiment of the present invention provides compounds according to formula I as described herein, wherein
R¹ is selected from cyanoalkyl, haloalkoxy, haloalkyl, cyclopropyl and oxetanyl;
R² is selected from alkoxy, H or halo;
X₁ is N and X₂ is CR⁴ and X₃ is N, or
X₁ is CR³, X₂ is CR⁴, and X₃ is N or CR⁵, or
X₁ is CR³, X₂ is N, and X₃ is CR⁵;
R³ is selected from alkoxy, H, halo, haloalkoxy;
R⁴ is selected from alkoxy, H, halo;
R⁵ is selected from H, halo;
W is selected from Ring A, Ring B, or Ring C

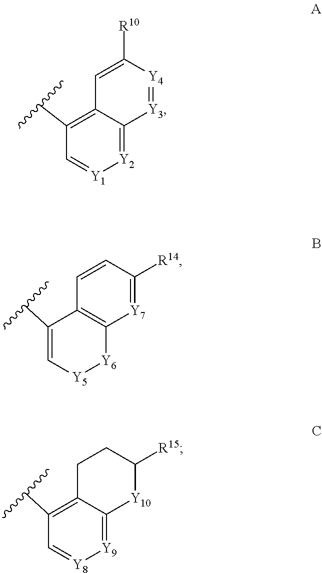

Y₁ is CR⁶, Y₂ is CR⁷, Y₃ is CR⁸ and Y₄ is CR⁹, or
Y₁ is N, Y₂ is CR⁷, Y₃ is CR⁸ and Y₄ is CR⁹, or
Y₁ is N, Y₂ is CR⁷, Y₃ is N and Y₄ is CR⁹, or
Y₁ is CR⁶, Y₂ is N, Y₃ is CR⁸ and Y₄ is CR⁹, or
Y₁ is CR⁶, Y₂ is CR⁷, Y₃ is N and Y₄ is CR⁹, or
Y₁ is CR⁶, Y₂ is CR⁷, Y₃ is CR⁸ and Y₄ is N;
R⁶ is H or alkylamino;
R⁷ is alkoxy, dimethylamino, H, methyl, methylamino, OH;
R⁸ is cyano, dimethylamino, H, halo;
R⁹ is alkoxy, cyclopropyl, haloalkyl dimethylamino, H, halo, alkyl;
R¹⁰ is H or halo;
Y₅ is NR¹¹, Y₆ is —C(═O)—, and Y₇ is CR¹³ or N, or
Y₅ is —C(═O)—, Y₆ is NR¹² and Y₇ is CR¹³;
R¹¹ is alkyl or H;
R¹² is alkyl or H;
R¹³ is H, or halo:
R¹⁴ is H or halo:
Y₈ is —CH—;
Y₉ is —CH—;
Y₁₀ is —CH₂— or —O—;
R¹⁵ is H, or alkyl;
wherein,
   when R² is H, then X₂ is CR⁴ and R⁴ is not H;
   when R¹ is haloalkyl, then R² is alkoxy; and
   when R¹ is haloalkoxy and R² is halo, then X₂ is N;
and pharmaceutically acceptable salts thereof.

An embodiment of the present invention provides compounds according to formula I as described herein, wherein
R¹ is selected from cyanoalkyl, haloalkoxy, or cyclopropyl;
R² is selected from alkoxy or halo;
X₁ is CR³, X₂ is CR⁴, and X₃ is N or CR⁵, or
X₁ is CR³, X₂ is N, and X₃ is CR⁵.
R³ is selected from alkoxy, halo, or haloalkoxy;
R⁴ is H;
R⁵ is H;
W is selected from Ring A, Ring B, or Ring C

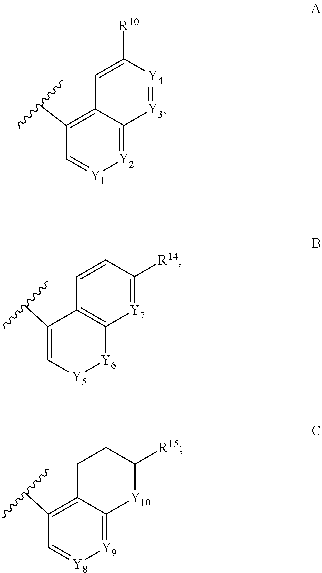

Y₁ is CR⁶, Y₂ is CR⁷, Y₃ is CR⁸ and Y₄ is CR⁹, or
Y₁ is N, Y₂ is CR⁷, Y₃ is CR⁸ and Y₄ is CR⁹, or
Y₁ is N, Y₂ is CR⁷, Y₃ is N and Y₄ is CR⁹, or
Y₁ is CR⁶, Y₂ is CR⁷, Y₃ is N and Y₄ is CR⁹.
R⁶ is H;
R⁷ is H or methylamino;
R⁸ is dimethylamino, H or halo;
R⁹ is cyclopropyl, H, halo or alkyl;
R¹⁰ is H;
Y₅ is NR¹¹, Y₆ is —C(═O)—, and Y₇ is CR¹³ or N, or
Y₅ is —C(═O)—, Y₆ is NR¹² and Y₇ is CR¹³;
R¹¹ is H;
R¹² is;
R¹³ is H, or halo:
R¹⁴ is halo:
Y₈ is —CH—;
Y₉ is —CH—;
Y₁₀ is —O—;
R¹⁵ is alkyl;
and pharmaceutically acceptable salts thereof.

An embodiment of the present invention provides compounds according to formula I as described herein, wherein
R¹ is selected from cyanoalkyl, haloalkoxy, or cyclopropyl;
R² is selected from alkoxy or halo;
X₁ is CR³, X₂ is CR⁴, and X₃ is N or CR⁵, or
X₁ is CR³, X₂ is N, and X₃ is CR⁵.
R³ is selected from alkoxy, halo, or haloalkoxy;
R⁴ is H;
R⁵ is H;
W is selected from Ring A, Ring B, or Ring C

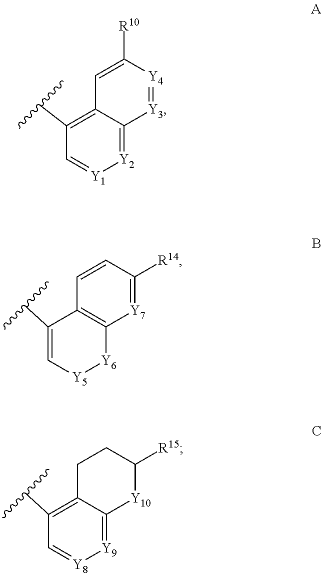

Y₁ is CR⁶, Y₂ is CR⁷, Y₃ is CR⁸ and Y₄ is CR⁹, or
Y₁ is N, Y₂ is CR⁷, Y₃ is CR⁸ and Y₄ is CR⁹, or
Y₁ is N, Y₂ is CR⁷, Y₃ is N and Y₄ is CR⁹, or
Y₁ is CR⁶, Y₂ is CR⁷, Y₃ is N and Y₄ is CR⁹.
R⁶ is H;
R⁷ is H or methylamino;
R⁸ is dimethylamino, H or halo;
R⁹ is cyclopropyl, H, halo or alkyl;
R¹⁰ is H;
Y₅ is NR¹¹, Y₆ is —C(═O)—, and Y₇ is CR¹³ or N, or
Y₅ is —C(═O)—, Y₆ is NR¹² and Y₇ is CR¹³;
R¹¹ is H;
R¹² is;
R¹³ is H, or halo:
R¹⁴ is halo:
Y₈ is —CH—;
Y₉ is —CH—;
Y₁₀ is —O—;
R¹⁵ is alkyl;
wherein,
   when R² is H, then X₂ is CR⁴ and R⁴ is not H;
   when R¹ is haloalkyl, then R² is alkoxy; and
   when R¹ is haloalkoxy and R² is halo, then X₂ is N;
and pharmaceutically acceptable salts thereof.

Particular examples of compounds of formula I as described herein are selected from
N-(4-(cyanomethyl)-2,5-difluorophenyl)naphthalene-1-sulfonamide;
N-[6-(2,2-difluoroethoxy)-5-fluoro-2-methoxy-3-pyridyl]naphthalene-1-sulfonamide;
2-chloro-N-[4-(cyanomethyl)-2,5-difluoro-phenyl]quinoline-5-sulfonamide;
2-chloro-N-(6-cyclopropyl-5-fluoro-2-methoxy-3-pyridyl)quinoline-5-sulfonamide;
2-chloro-N-[6-(cyanomethyl)-5-fluoro-2-methoxy-3-pyridyl]quinoline-5-sulfonamide;
2-chloro-N-[6-(difluoromethoxy)-5-fluoro-2-methoxy-3-pyridyl]quinoline-5-sulfonamide;
2-chloro-N-[5-fluoro-2-methoxy-6-(oxetan-3-yl)-3-pyridyl]quinoline-5-sulfonamide;
2-chloro-N-[5-(2,2-difluoroethyl)-4,6-dimethoxy-pyrimidin-2-yl]quinoline-5-sulfonamide;
2-chloro-N-[5-(2,2-difluoroethoxy)-3-fluoro-6-methoxy-2-pyridyl]quinoline-5-sulfonamide;
2-chloro-N-[6-(2,2-difluoroethoxy)-5-fluoro-2-methoxy-3-pyridyl]quinoline-5-sulfonamide;
N-[2,6-bis(difluoromethoxy)-5-fluoro-3-pyridyl]-2-chloro-quinoline-5-sulfonamide;
7-chloro-N-[4-(cyanomethyl)-2,5-difluoro-phenyl]isoquinoline-4-sulfonamide;
7-chloro-N-[6-(difluoromethoxy)-5-fluoro-2-methoxy-3-pyridyl]isoquinoline-4-sulfonamide;
7-chloro-N-[5-(2,2-difluoroethoxy)-3-fluoro-6-methoxy-2-pyridyl]isoquinoline-4-sulfonamide;
7-chloro-N-[6-(2,2-difluoroethoxy)-5-fluoro-2-methoxy-3-pyridyl]isoquinoline-4-sulfonamide;
N-[6-(2,2-difluoroethoxy)-5-fluoro-2-methoxy-3-pyridyl]-2-methyl-chromane-5-sulfonamide;
5-chloro-N-[4-(cyanomethyl)-2,5-difluoro-phenyl]naphthalene-1-sulfonamide;
5-chloro-N-[6-(2,2-difluoroethoxy)-5-fluoro-2-methoxy-3-pyridyl]naphthalene-1-sulfonamide;
7-chloro-N-[6-(2,2-difluoroethoxy)-5-fluoro-2-methoxy-3-pyridyl]naphthalene-1-sulfonamide;
8-chloro-N-[4-(cyanomethyl)-2,5-difluoro-phenyl]-1-hydroxy-isoquinoline-4-sulfonamide;
7-chloro-N-[4-(cyanomethyl)-2,5-difluoro-phenyl]-1-keto-2H-isoquinoline-4-sulfonamide;
7-chloro-N-[5-(2-fluoroethoxy)-4-methoxy-pyrimidin-2-yl]-1-keto-2H-isoquinoline-4-sulfonamide;
7-chloro-N-[6-(difluoromethoxy)-5-fluoro-2-methoxy-3-pyridyl]-1-keto-2H-isoquinoline-4-sulfonamide;
7-chloro-N-[5-(2,2-difluoroethyl)-4,6-dimethoxy-pyrimidin-2-yl]-1-keto-2H-isoquinoline-4-sulfonamide;
7-chloro-N-[5-(2,2-difluoroethoxy)-3-fluoro-6-methoxy-2-pyridyl]-1-oxo-2H-isoquinoline-4-sulfonamide;
7-chloro-N-[4-(2,2-difluoroethoxy)-5-fluoro-2-methoxy-phenyl]-1-keto-2H-isoquinoline-4-sulfonamide;
N-[2,6-bis(difluoromethoxy)-5-fluoro-3-pyridyl]-7-chloro-1-keto-2H-isoquinoline-4-sulfonamide;
2-chloro-N-[4-(cyanomethyl)-2,5-difluoro-phenyl]-8-oxo-7H-1,7-naphthyridine-5-sulfonamide;
2-chloro-N-[6-(2,2-difluoroethoxy)-5-fluoro-2-methoxy-3-pyridyl]-8-keto-7H-1,7-naphthyridine-5-sulfonamide;
7-chloro-N-[6-(2,2-difluoroethoxy)-5-fluoro-2-methoxy-3-pyridyl]-2-keto-1H-quinoline-4-sulfonamide;
N-[6-(2,2-difluoroethoxy)-5-fluoro-2-methoxy-3-pyridyl]-2-(dimethylamino)quinoline-5-sulfonamide;
N-[4-(cyanomethyl)-2,5-difluoro-phenyl]-2-(dimethylamino)quinoline-5-sulfonamide;
N-[6-(2,2-difluoroethoxy)-5-fluoro-2-methoxy-3-pyridyl]-2-methyl-quinoline-5-sulfonamide;
6-chloro-N-[6-(2,2-difluoroethoxy)-5-fluoro-2-methoxy-3-pyridyl]naphthalene-1-sulfonamide;
N-[6-(2,2-difluoroethoxy)-5-fluoro-2-methoxy-3-pyridyl]-2-(difluoromethyl)quinoline-5-sulfonamide;
7-chloro-N-[6-(2,2-difluoroethoxy)-5-fluoro-2-methoxy-3-pyridyl]-1-keto-2-methyl-isoquinoline-4-sulfonamide;
7-chloro-N-[6-(2,2-difluoroethoxy)-5-fluoro-2-methoxy-3-pyridyl]-2-keto-1-methyl-quinoline-4-sulfonamide;
7,8-dichloro-N-[4-(cyanomethyl)-2,5-difluoro-phenyl]-1-keto-2H-isoquinoline-4-sulfonamide;
7,8-dichloro-N-[6-(2,2-difluoroethoxy)-5-fluoro-2-methoxy-3-pyridyl]-1-keto-2H-isoquinoline-4-sulfonamide;
N-[6-(2,2-difluoroethoxy)-5-fluoro-2-methoxy-3-pyridyl]-5-(dimethylamino)naphthalene-1-sulfonamide;
7-chloro-N-[6-(2,2-difluoroethoxy)-5-fluoro-2-methoxy-3-pyridyl]-1-(dimethylamino)isoquinoline-4-sulfonamide;
7-chloro-N-[4-(cyanomethyl)-2,5-difluoro-phenyl]-1-(dimethylamino)isoquinoline-4-sulfonamide;
7-chloro-N-[6-(2,2-difluoroethoxy)-5-fluoro-2-methoxy-3-pyridyl]-1-(methylamino)isoquinoline-4-sulfonamide;
7-chloro-N-[4-(cyanomethyl)-2,5-difluoro-phenyl]-1-(methylamino)isoquinoline-4-sulfonamide;
7-chloro-N-[6-(2,2-difluoroethoxy)-5-fluoro-2-methoxy-3-pyridyl]-2-(methylamino)quinoline-4-sulfonamide;
7-chloro-8-cyano-N-[6-(2,2-difluoroethoxy)-5-fluoro-2-methoxy-3-pyridyl]isoquinoline-4-sulfonamide;
7-chloro-N-[6-(difluoromethoxy)-5-fluoro-2-methoxy-3-pyridyl]quinoline-4-sulfonamide;
7-chloro-N-[4-(cyanomethyl)-2,5-difluoro-phenyl]-1-methoxy-isoquinoline-4-sulfonamide;
2-chloro-N-[6-(2,2-difluoroethoxy)-5-fluoro-2-methoxy-3-pyridyl]-1,7-naphthyridine-5-sulfonamide;
2-chloro-N-[5-(difluoromethoxy)-4,6-dimethoxy-pyrimidin-2-yl]quinoline-5-sulfonamide;
N-(4-(cyanomethyl)-2,5-difluorophenyl)-1-oxo-1,2-dihydroisoquinoline-4-sulfonamide;
7-chloro-N-[4-(cyanomethyl)-2,5-difluoro-phenyl]quinoline-4-sulfonamide;
2-bromo-N-[6-(2,2-difluoroethoxy)-5-fluoro-2-methoxy-3-pyridyl]quinoline-5-sulfonamide;
2-cyclopropyl-N-[6-(2,2-difluoroethoxy)-5-fluoro-2-methoxy-3-pyridyl]quinoline-5-sulfonamide;
N-[6-(2,2-difluoroethoxy)-5-fluoro-2-methoxy-3-pyridyl]-2-methoxy-quinoline-5-sulfonamide;
N-[6-(2,2-difluoroethoxy)-5-fluoro-2-methoxy-3-pyridyl]-1-(dimethylamino)isoquinoline-5-sulfonamide;
N-[6-(2,2-difluoroethoxy)-5-fluoro-2-methoxy-3-pyridyl]tetralin-5-sulfonamide;
and pharmaceutically acceptable salts thereof.
Preferred examples of compounds of formula I as described herein are selected from
2-chloro-N-(6-cyclopropyl-5-fluoro-2-methoxy-3-pyridyl)quinoline-5-sulfonamide;
2-chloro-N-[6-(cyanomethyl)-5-fluoro-2-methoxy-3-pyridyl]quinoline-5-sulfonamide;
2-chloro-N-[6-(difluoromethoxy)-5-fluoro-2-methoxy-3-pyridyl]quinoline-5-sulfonamide;
2-chloro-N-[6-(2,2-difluoroethoxy)-5-fluoro-2-methoxy-3-pyridyl]quinoline-5-sulfonamide;
7-chloro-N-[6-(2,2-difluoroethoxy)-5-fluoro-2-methoxy-3-pyridyl]isoquinoline-4-sulfonamide;
N-[6-(2,2-difluoroethoxy)-5-fluoro-2-methoxy-3-pyridyl]-2-methyl-chromane-5-sulfonamide;
5-chloro-N-[6-(2,2-difluoroethoxy)-5-fluoro-2-methoxy-3-pyridyl]naphthalene-1-sulfonamide;
7-chloro-N-[6-(difluoromethoxy)-5-fluoro-2-methoxy-3-pyridyl]-1-keto-2H-isoquinoline-4-sulfonamide;
7-chloro-N-[5-(2,2-difluoroethoxy)-3-fluoro-6-methoxy-2-pyridyl]-1-oxo-2H-isoquinoline-4-sulfonamide;
7-chloro-N-[4-(2,2-difluoroethoxy)-5-fluoro-2-methoxy-phenyl]-1-keto-2H-isoquinoline-4-sulfonamide;
N-[2,6-bis(difluoromethoxy)-5-fluoro-3-pyridyl]-7-chloro-1-keto-2H-isoquinoline-4-sulfonamide;
2-chloro-N-[6-(2,2-difluoroethoxy)-5-fluoro-2-methoxy-3-pyridyl]-8-keto-7H-1,7-naphthyridine-5-sulfonamide;
7-chloro-N-[6-(2,2-difluoroethoxy)-5-fluoro-2-methoxy-3-pyridyl]-2-keto-1H-quinoline-4-sulfonamide;
N-[6-(2,2-difluoroethoxy)-5-fluoro-2-methoxy-3-pyridyl]-2-methyl-quinoline-5-sulfonamide;
6-chloro-N-[6-(2,2-difluoroethoxy)-5-fluoro-2-methoxy-3-pyridyl]naphthalene-1-sulfonamide;
7,8-dichloro-N-[6-(2,2-difluoroethoxy)-5-fluoro-2-methoxy-3-pyridyl]-1-keto-2H-isoquinoline-4-sulfonamide;
N-[6-(2,2-difluoroethoxy)-5-fluoro-2-methoxy-3-pyridyl]-5-(dimethylamino)naphthalene-1-sulfonamide;
7-chloro-N-[6-(2,2-difluoroethoxy)-5-fluoro-2-methoxy-3-pyridyl]-1-(methylamino)isoquinoline-4-sulfonamide;
2-chloro-N-[6-(2,2-difluoroethoxy)-5-fluoro-2-methoxy-3-pyridyl]-1,7-naphthyridine-5-sulfonamide;
2-bromo-N-[6-(2,2-difluoroethoxy)-5-fluoro-2-methoxy-3-pyridyl]quinoline-5-sulfonamide;
2-cyclopropyl-N-[6-(2,2-difluoroethoxy)-5-fluoro-2-methoxy-3-pyridyl]quinoline-5-sulfonamide;
   and pharmaceutically acceptable salts thereof.

Processes for the manufacture of compounds of formula I as described herein are an object of the invention.

The present compounds of formula I and their pharmaceutically acceptable salts can be prepared by methods known in the art, for example, by processes described below, which process comprises reacting a compound of formula III with a compound of formula II in the presence of a base selected from N-ethyldiisopropylamine, pyridine, potassium phosphate or sodium hydride to provide a compound of formula I,

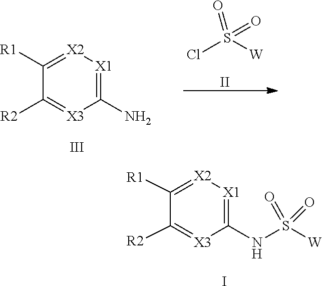

wherein R¹, R², R³, X₁, X₂, X₃ and W are as described above.

### General Synthetic Schemes

The compounds of formula I may be prepared in accordance with the process variant described above and with the following scheme 1. The starting materials are commercially available or may be prepared in accordance with known methods.

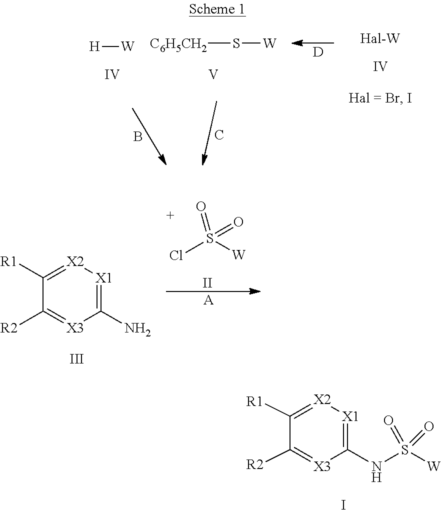

Compounds of general formula I can be prepared by reacting sulfonylchloride II with amines III in the presence of a base like N-ethyldiisopropylamine, pyridine, potassium phosphate or sodium hydride. Sulfonylchlorides II can be prepared from intermediate IV in the presence of chlorosulfonylating agent like chlorosulfonic acid or in the presence of sulfonylating agent like sulfur trioxide N,N-dimethylformamide complex, followed by chlorination of the intermediate sulfonic acid with a chlorinating agent like thionylchloride or oxalylchloride (step B). Furthermore, sulfonylchlorides II can be prepared by oxydative chlorination of intermediates V with N-chlorosuccinimide in a mixture of an organic solvent such as acetic acid and water (step C). Intermediates V are available by reaction of iodo- or bromo-compounds VI with benzylmercaptane using Buchwald-Hartwig type cross coupling using palladium catalyst system such as Pd(OAc)₂ or Pd₂(dba)₃/Xantphos or Xphos and a base such as DIPEA or Cs₂CO₃ at elevated temperatures in solvents such as dioxane or toluene (step D). The starting materials are commercially available or may be prepared in accordance with known methods.

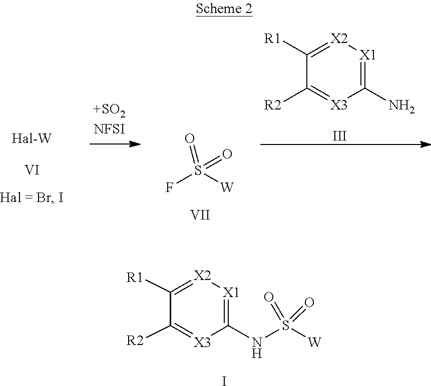

Furthermore, compounds of general formula I can be prepared by reacting compound VI with sulfur dioxide and NFSI (N-fluorobenzenesulfonimide) to give sulfonylfluoride VII which is then reacted with amine III in presence of a base such as LiHMDS or LDA to obtain I as the final product of this sequence (Scheme 2).

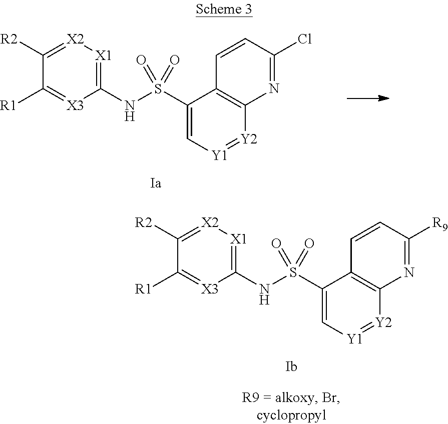

Compounds of general formula Ib can be prepared from compound Ia by reacting with a suitable reagent to substitute the chlorine atom (Scheme 3). To produce a compound with R9 is MeO, sodium methoxide in methanol can be used. If a compound with R9=Br is desired, reaction of Ia with trimethylsilylbromide can be used. To synthesise a compound Ib with R9=cyclopropyl, Ia can be reacted with cycylopropylboronic acid and a palladium catalyst.

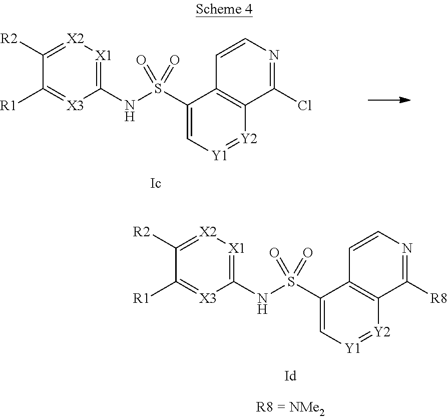

Compounds of general formula Id can be prepared by reacting compound Ic with dimethylamine or its salts in presence of a base in a suitable solvent like ethanol at elevated temperatures (Scheme 4).

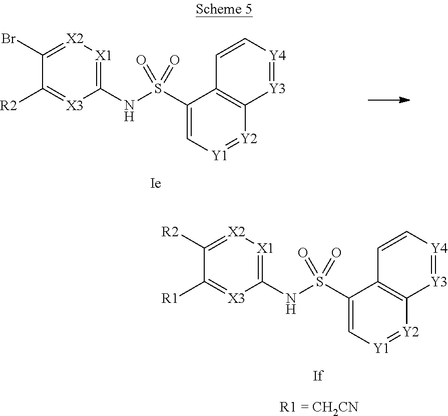

Compounds of general formula If can be prepared by reacting compound Ie with a suitable reagent such as 4-isoxazoleboronic acid pinacol ester, a base like potassium fluoride and a palladium catalyst such as Pd(dppf)Cl₂ in DMSO at elevated temperatures (Scheme 5).

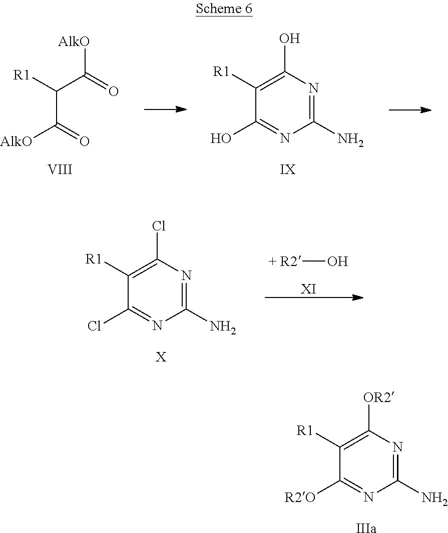

Aminopyrimidines IIIa can be prepared by reaction of malonester derivative VIII with guanidine hydrochloride and a base like sodium methoxide to give compound IX which is then reacted with an halogenating agent like phosphorous oxychloride to form compound X, followed by reaction with alkohol XI and a base like sodium hydride to obtain IIIa (Scheme 6).

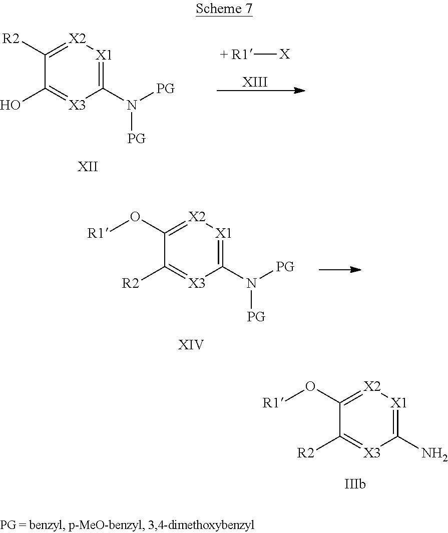

Amines IIIb may be prepared by alkylation of compounds XII with an alkylating reagent XIII (X is a leaving group such as iodide, bromide, methanesulfonate, trifluoromethanesulfonate, phosphonate or the like) and a base such as sodium hydride, potassium tert-butoxide, potassium carbonate or cesium carbonate in a suitable solvent such as N,N-dimethylformamide, N,N-dimethylacetamide, N-methylpyrrolidone or tetrahydrofuran followed by removal of the protecting group using a known method such as acidic or hydrogenolytic cleavage. A preferred protecting group PG is p-methoxybenzyl which can be removed by treatment with trifluoroacetic acid at room temperature or at elevated temperatures with or without a solvent such as dichloromethane.

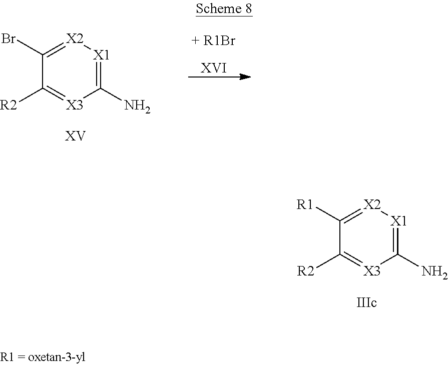

Amines IIIc may be prepared by reaction of compounds XV with reagent XVI using a photocatalytic reaction (Scheme 8). A preferred condition is the use of an iridium and a nickel catalyst in ethyleneglycol dimethylether and irradiation at 450 nM wavelength.

Another embodiment of the invention provides a pharmaceutical composition or medicament containing a compound of the invention and a therapeutically inert carrier, diluent or excipient, as well as a method of using the compounds of the invention to prepare such composition and medicament. In one example, the compound of formula I may be formulated by mixing at ambient temperature at the appropriate pH, and at the desired degree of purity, with physiologically acceptable carriers, i.e., carriers that are non-toxic to recipients at the dosages and concentrations employed into a galenical administration form. The pH of the formulation depends mainly on the particular use and the concentration of compound, but preferably ranges anywhere from about 3 to about 8. In one example, a compound of formula I is formulated in an acetate buffer, at pH 5. In another embodiment, the compound of formula I is sterile. The compound may be stored, for example, as a solid or amorphous composition, as a lyophilized formulation or as an aqueous solution.

Compositions are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

The compounds of the invention may be administered by any suitable means, including oral, topical (including buccal and sublingual), rectal, vaginal, transdermal, parenteral, subcutaneous, intraperitoneal, intrapulmonary, intradermal, intrathecal and epidural and intranasal, and, if desired for local treatment, intralesional administration. Parenteral infusions include intramuscular, intravenous, intraarterial, intraperitoneal, or subcutaneous administration.

The compounds of the present invention may be administered in any convenient administrative form, e.g., tablets, powders, capsules, solutions, dispersions, suspensions, syrups, sprays, suppositories, gels, emulsions, patches, etc. Such compositions may contain components conventional in pharmaceutical preparations, e.g., diluents, carriers, pH modifiers, sweeteners, bulking agents, and further active agents.

A typical formulation is prepared by mixing a compound of the present invention and a carrier or excipient. Suitable carriers and excipients are well known to those skilled in the art and are described in detail in, e.g., Ansel, Howard C., et al., Ansel's Pharmaceutical Dosage Forms and Drug Delivery Systems. Philadelphia: Lippincott, Williams & Wilkins, 2004; Gennaro, Alfonso R., et al. Remington: The Science and Practice of Pharmacy. Philadelphia: Lippincott, Williams & Wilkins, 2000; and Rowe, Raymond C. Handbook of Pharmaceutical Excipients. Chicago, Pharmaceutical Press, 2005. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

The compounds of formula I and their pharmaceutically acceptable salts can be processed with pharmaceutically inert, inorganic or organic adjuvants for the production of tablets, coated tablets, dragées, hard gelatin capsules, injection solutions or topical formulations Lactose, corn starch or derivatives thereof, talc, stearic acid or its salts etc. can be used, for example, as such adjuvants for tablets, dragées and hard gelatin capsules.

Suitable adjuvants for soft gelatin capsules, are, for example, vegetable oils, waxes, fats, semi-solid substances and liquid polyols, etc.

Suitable adjuvants for the production of solutions and syrups are, for example, water, polyols, saccharose, invert sugar, glucose, etc.

Suitable adjuvants for injection solutions are, for example, water, alcohols, polyols, glycerol, vegetable oils, etc.

Suitable adjuvants for suppositories are, for example, natural or hardened oils, waxes, fats, semi-solid or liquid polyols, etc.

Suitable adjuvants for topical ocular formulations are, for example, cyclodextrins, mannitol or many other carriers and excipients known in the art.

Moreover, the pharmaceutical preparations can contain preservatives, solubilizers, viscosity-increasing substances, stabilizers, wetting agents, emulsifiers, sweeteners, colorants, flavorants, salts for varying the osmotic pressure, buffers, masking agents or antioxidants. They can also contain still other therapeutically valuable substances.

The dosage can vary in wide limits and will, of course, be fitted to the individual requirements in each particular case. In general, in the case of oral administration a daily dosage of about 0.1 mg to 20 mg per kg body weight, preferably about 0.5 mg to 4 mg per kg body weight (e.g. about 300 mg per person), divided into preferably 1-3 individual doses, which can consist, for example, of the same amounts, should it be appropriate. In the case of topical administration, the formulation can contain 0.001% to 15% by weight of medicament and the required dose, which can be between 0.1 and 25 mg in can be administered either by single dose per day or per week, or by multiple doses (2 to 4) per day, or by multiple doses per week It will, however, be clear that the upper or lower limit given herein can be exceeded when this is shown to be indicated.

The invention also relates in particular to:

A compound of formula I for use as therapeutically active substance;

A compound of formula I for use in the treatment of a disease modulated by GPR17;

Likewise an object of the present invention is a pharmaceutical composition comprising a compound according to formula I as described herein and a therapeutically inert carrier.

The use of a compound of formula I for the treatment or prophylaxis of conditions resulting from direct damage to myelin sheaths (including but not limited central pontine and extra-pontine myelinolysis, carbon monoxide poisoning, nutritional deficiency, and virus-induced demyelination), demyelinating disorders (including but not limited to multiple sclerosis, acute and multiphasic disseminated encephalomyelitis, neuromyelitis optica spectrum disorders, and leukodystrophies), CNS disorders associated with myelin loss (including but not limited to Alzheimer's disease, schizophrenia, Parkinson's disease, Huntington's disease, Amyotrophic lateral sclerosis, and Ischemia due to stroke), and Inflammation in the CNS for instance following encephalitis, primary angiitis, meningitis and obesity.

An embodiment of the present invention is the use of a compound of formula I for the treatment or prophylaxis of multiple sclerosis, Alzheimer's disease, Parkinson's disease, or Huntington's disease.

A particular embodiment of the invention is the use of a compound of formula I for the treatment or prophylaxis of multiple sclerosis.

The use of a compound of formula I for the preparation of a medicament for the treatment or prophylaxis of conditions resulting from direct damage to myelin sheaths (including but not limited central pontine and extra-pontine myelinolysis, carbon monoxide poisoning, nutritional deficiency, and virus-induced demyelination), demyelinating disorders (including but not limited to multiple sclerosis, acute and multiphasic disseminated encephalomyelitis, neuromyelitis optica spectrum disorders, and leukodystrophies), CNS disorders associated with myelin loss (including but not limited to Alzheimer's disease, schizophrenia, Parkinson's disease, Huntington's disease, Amyotrophic lateral sclerosis, and Ischemia due to stroke), and Inflammation in the CNS for instance following encephalitis, primary angiitis, meningitis and obesity.

An embodiment of the present invention is the use of a compound of formula I for the preparation of a medicament for the treatment or prophylaxis of multiple sclerosis, Alzheimer's disease, Parkinson's disease, or Huntington's disease.

A particular embodiment of the invention is the use of a compound of formula I for the preparation of a medicament for the treatment or prophylaxis of multiple sclerosis.

A compound according to formula I for use in the treatment or prophylaxis of conditions resulting from direct damage to myelin sheaths (including but not limited central pontine and extra-pontine myelinolysis, carbon monoxide poisoning, nutritional deficiency, and virus-induced demyelination), demyelinating disorders (including but not limited to multiple sclerosis, acute and multiphasic disseminated encephalomyelitis, neuromyelitis optica spectrum disorders, and leukodystrophies), CNS disorders associated with myelin loss (including but not limited to Alzheimer's disease, schizophrenia, Parkinson's disease, Huntington's disease, Amyotrophic lateral sclerosis, and Ischemia due to stroke), and Inflammation in the CNS for instance following encephalitis, primary angiitis, meningitis and obesity.

An embodiment of the present invention is a compound of formula I for use in the treatment or prophylaxis of multiple sclerosis, Alzheimer's disease, Parkinson's disease, or Huntington's disease.

A particular embodiment of the invention is a compound according to formula I for use in the treatment or prophylaxis of multiple sclerosis.

A method for the treatment or propylaxis of conditions resulting from direct damage to myelin sheaths (including but not limited central pontine and extra-pontine myelinolysis, carbon monoxide poisoning, nutritional deficiency, and virus-induced demyelination), demyelinating disorders (including but not limited to multiple sclerosis, acute and multiphasic disseminated encephalomyelitis, neuromyelitis optica spectrum disorders, and leukodystrophies), CNS disorders associated with myelin loss (including but not limited to Alzheimer's disease, schizophrenia, Parkinson's disease, Huntington's disease, Amyotrophic lateral sclerosis, and Ischemia due to stroke), and Inflammation in the CNS for instance following encephalitis, primary angiitis, meningitis and obesity, which method comprises administering an effective amount of a compound of formula I to a patient in need thereof.

An embodiment of the present invention is a method for the treatment or prophylaxis of multiple sclerosis, Alzheimer's disease, Parkinson's disease, or Huntington's disease, which method comprises administering an effective amount of a compound of formula I to a patient in need thereof.

A particular embodiment of the invention is a method for the treatment or prophylaxis of multiple sclerosis, which method comprises administering an effective amount of a compound of formula I to a patient in need thereof.

Also an embodiment of the present invention provides compounds of formula I as described herein, when manufactured according to any one of the described processes.

### Assay Procedures

GPR17 cAMP Assay Protocol:

CHO-K1 cells stably expressing vector containing untagged human GPR17 short isoform (Roche) were cultured at 37° C./5% CO2 in DMEM (Dulbecco's Modified Eagle Medium):F-12 (1:1) supplemented with 10% foetal bovine serum and 400 μg/ml Geneticin.

Changes in intracellular cyclic adenosine monophosphate (cAMP) levels were quantified using the Nano-TRF Detection Assay kit (Roche Diagnostics, Cat. No. 05214386001). This assay allows for direct cAMP quantification in a homogeneous solution. cAMP is detected based on time-resolved fluorescence energy transfer (TR-FRET) and competitive binding of ruthenylated cAMP and endogenous cAMP to an anti-cAMP monoclonal antibody labeled with AlexaFluor-700. The Ruthenium complex serves as the FRET donor and transfers energy to AlexaFluor-700. The FRET signal is inversely proportional to the cAMP concentration.

CHO-GPR17S cells were detached with Accutase and resuspended in assay buffer consisting of Hank's Balanced Salt Solution (HBSS), 10 mM HEPES (4-(2-hydroxyethyl) piperazine-1-ethanesulfonic acid solution) and 0.1% bovine serum albumin (pH 7.4). The cells were seeded in black 384-well plates (Corning) at a density of 10′000 cells/20 μl assay buffer until the addition of compounds.

Test antagonist compounds were serially diluted in dimethyl sulfoxide (DMSO) and spotted in 384-well plates. The compounds were then diluted in BSS buffer supplemented with an EC80 concentration of MDL29,951 (3-(2-Carboxy-4,6-dichloroindol-3-yl)propionic acid) (GPR17 agonist) plus 3-Isobutyl-1-methylxanthine (IBMX) (0.5 mM final concentration) and added to the cells at room temperature. Forskolin (15 μM final concentration) was added 5 minutes after the test compounds and the cells were incubated at room temperature for 30 minutes. The assay was stopped by adding cAMP detection mix (containing detergents for cell lysis) for 90 minutes at room temperature.

Cellular cAMP was measured using a Paradigm reader (Molecular Devices). The raw data was used to calculate the FRET signal based on the assay's P-factor as per cAMP kit instructions. The data was normalized to the maximal activity of a reference antagonist and dose response curves were fitted to the percent activity of the test compounds using a sigmoidal dose response model (Genedata Screener).

Results in the hGPR17 cAMP assay are provided for compounds of formula I in Table 1

TABLE 1

| | hGPR17 |
|---|---|
| Example | cAMP |
| number | IC50 [μM] |
| 1 | 0.282 |
| 2 | 0.085 |
| 3 | 0.217 |
| 4 | 0.045 |
| 5 | 0.019 |
| 6 | 0.049 |
| 7 | 0.431 |
| 8 | 0.174 |
| 9 | 0.495 |
| 10 | 0.028 |
| 11 | 0.111 |
| 12 | 0.260 |
| 13 | 0.092 |
| 14 | 0.415 |
| 15 | 0.011 |
| 16 | 0.031 |
| 17 | 0.088 |
| 18 | 0.043 |
| 19 | 0.089 |
| 20 | 0.327 |
| 21 | 0.174 |
| 22 | 0.331 |
| 23 | 0.019 |
| 24 | 0.278 |
| 25 | 0.050 |
| 26 | 0.005 |
| 27 | 0.032 |
| 28 | 0.955 |
| 29 | 0.030 |
| 30 | 0.035 |
| 31 | 0.065 |
| 32 | 0.238 |
| 33 | 0.011 |
| 34 | 0.048 |
| 35 | 0.214 |
| 36 | 0.371 |
| 37 | 0.246 |
| 38 | 0.182 |
| 39 | 0.007 |
| 40 | 0.013 |
| 41 | 0.061 |
| 42 | 0.423 |
| 43 | 0.035 |
| 44 | 0.315 |
| 45 | 0.072 |
| 46 | 0.253 |
| 47 | 0.933 |
| 48 | 0.154 |
| 49 | 0.905 |
| 50 | 0.038 |
| 51 | 0.448 |
| 52 | 0.594 |
| 53 | 0.976 |
| 54 | 0.009 |

The invention will now be illustrated by the following examples which have no limiting character.

In case the preparative examples are obtained as a mixture of enantiomers, the pure enantiomers can be obtained by methods described herein or by methods known to those skilled in the art, such as e.g. chiral chromatography or crystallization.

### EXAMPLES

All examples and intermediates were prepared under nitrogen atmosphere if not specified otherwise.

### Intermediates A

### Intermediate A1: 1-naphthalenesulfonyl chloride

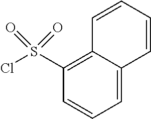 [SMILES: O=S(=O)(Cl)c1cccc2ccccc12]

### Intermediate A1 is commercial (CAS: 85-46-1)

### Intermediate A2: 7-chloroquinoline-4-sulfonyl fluoride

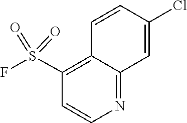 [SMILES: O=S(=O)(F)c1ccnc2cc(Cl)ccc12]

In a three-necked flask, n-butyllithium, (1.6M solution in hexanes (2.0 ml, 4.95 mmol) was added dropwise to the solution of 4-bromo-7-chloro-quinoline (CAS 98519-65-4, 1.0 g, 4.12 mmol, 1.0 eq) in THE (20 ml) at −78° C. under argon. After 15 min of stirring sulfur dioxide (2.64 g, 41.24 mmol) was added thereto dropwise as a solution in THE (10 ml). The resulting mixture was warmed slowly to room temperature and stirred for 3 h. Precipitation of sulfinate salt was observed during this period. After cooling the resulting mixture to 0° C. N-fluorobenzenesulfonimide (1.43 mg, 4.54 mmol, 1.1 eq) was added as one portion and the resulting mixture was stirred for another 3 h. The resulting mixture was poured into brine (20 ml) and extracted with ethyl acetate (2×20 ml). The combined organic phase was evaporated. The residue was subjected to column chromatography on silica gel (heptanes/ethyl acetate) to afford 7-chloroquinoline-4-sulfonyl fluoride (103.9 mg, 0.42 mmol, 10.3% yield) as a yellow solid 1H NMR (500 MHz, CDCl₃) δ=9.19 (br s, 1H), 8.43 (br d, J=9.1 Hz, 1H), 8.31 (br s, 1H), 8.10 (br d, J=3.0 Hz, 1H), 7.79 (br d, J=9.1 Hz, 1H).

### Intermediate A3: 2-chloroquinoline-5-sulfonyl chloride

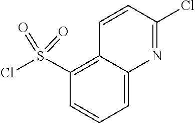 [SMILES: O=S(=O)(Cl)c1cccc2nc(Cl)ccc12]

2-Chloroquinoline (4 g, 24.45 mmol) was dissolved in chlorosulfonic acid (37.41 g, 21.5 ml, 318 mmol). The reaction mixture was heated to 140° C. and stirred 2 days, then cooled to room temperature and carefully quenched into ice/ethyl acetate. The organic layer was separated and the aqueous layer was re-extracted with ethyl acetate. The combined organic layers were dried over Na2SO4, filtered and concentrated in vacuo. The crude material was purified by flash chromatography on silica gel (120 g, 0% to 10% ethyl acetate in heptane) to afford 2-chloroquinoline-5-sulfonyl chloride (383 mg, 6.0%) as white solid MS (ESI) m/z: 262.0 [M+H]+; 1H NMR (300 MHz, DMSO-d6) δ=9.21 (dd, J=0.8, 8.9 Hz, 1H), 8.00 (dd, J=1.4, 7.3 Hz, 1H), 7.92 (td, J=1.1, 8.3 Hz, 1H), 7.78-7.72 (m, 1H), 7.64 (d, J=8.9 Hz, 1H), and 2-chloroquinoline-6-sulfonyl chloride (406 mg, 6.2%) as white solid MS (ESI) m/z: 262.0 [M+H]+; 1H NMR (300 MHz, DMSO-d6) δ=8.55 (d, J=8.5 Hz, 1H), 8.28 (d, J=1.8 Hz, 1H), 7.99 (dd, J=1.9, 8.8 Hz, 1H), 7.93-7.88 (m, 1H), 7.61 (d, J=8.7 Hz, 1H).

### Intermediate A4: 7-chloroisoquinoline-4-sulfonyl chloride

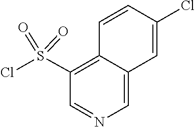 [SMILES: O=S(=O)(Cl)c1cncc2cc(Cl)ccc12]

### Step 1: 4-(benzylthio)-7-chloro-isoquinoline

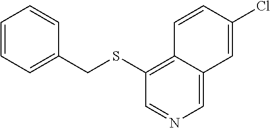 [SMILES: Clc1ccc2c(SCc3ccccc3)cncc2c1]

A mixture of 4-bromo-7-chloro-isoquinoline (250 mg, 1.03 mmol), benzyl mercaptane (141 mg, 134 ul, 1.13 mmol), N,N-diethylisopropylamine (266 mg, 360 ul, 2.06 mmol), 9,9-dimethyl-4,5-bis(diphenylphosphino)xanthene (30 mg, 0.052 mmol) and tris(dibenzylideneacetone)dipalladium(0) (28 mg, 0.031 mmol) in 1,4-dioxane (2 ml) was heated to 80° C. and stirred for 1.5 h. The reaction mixture was filtered and washed (2×10 ml ethyl acetate) the filtrate was extracted with water/ethyl acetate. The organic layers were dried (Na2SO4) and concentrated in vacuo. The crude material was purified by flash chromatography on silica gel (25 g, 20% to 100% ethyl acetate in heptane) and the collected fractions was evaporated to provide 4-(benzylthio)-7-chloro-isoquinoline (282 mg, 95.71%) as yellow oil. MS (ESI) m/z: 286.1 [M+H]+

### Step 2: 7-chloroisoquinoline-4-sulfonyl chloride

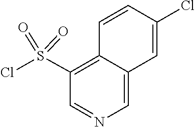 [SMILES: O=S(=O)(Cl)c1cncc2cc(Cl)ccc12]

To a solution of 4-(benzylthio)-7-chloro-isoquinoline (282 mg, 0.99 mmol) in acetic acid (3 ml) and water (0.3 ml) was added N-chlorosuccinimide (395 mg, 2.96 mmol) in portions over 15 min at room temperature. The clear solution was stirred for 2 h at room temperature, then the solvent was evaporated. The residue was extracted 2×with ethyl acetate and ice water, the organic layers were dried with MgSO4, filtered and concentrated under vacuum to afford 7-chloroisoquinoline-4-sulfonyl chloride (259 mg, 100%) as yellow solid, MS: 262.0 [M+H]+ ESI pos.

### Intermediate A5: 1-oxo-2H-isoquinoline-4-sulfonyl chloride

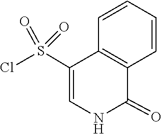 [SMILES: O=c1[nH]cc(S(=O)(=O)Cl)c2ccccc12]

To isoquinolin-1(2H)-one (100 mg, 0.69 mmol) under nitrogen was added chlorosulfonic acid (496 mg, 285 μl, 4.13 mmol). The reaction mixture was stirred for 2 h at 95° C. The mixture was cooled to 20° C. then ice (10 g) was added carefully. The grey-brown suspension was transfer into a plastic tube and centrifuged twice (water was decanted). The solid residue was transferred with ethyl acetate/acetonitrile into a flask and the solvent was evaporated to afford 1-oxo-1,2-dihydroisoquinoline-4-sulfonyl chloride (74 mg, 0.304 mmol, 44.1% yield) MS: 244.0 [M+H]+ ESI pos.

### Intermediate A6: 2-methylchromane-5-sulfonyl chloride

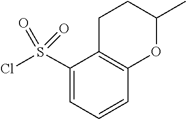

### Step 1: 4-(2-bromo-6-hydroxy-phenyl)but-3-en-2-one

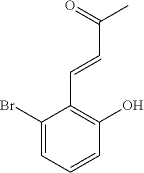

To a solution of 2-bromo-6-hydroxybenzaldehyde (1.0 g, 4.97 mmol) in acetone (6 ml) was added slowly at 25° C. NaOH (1M in water, 9.0 ml, 9.0 mmol) and stirred for 16 h. The mixture was acidified by dropwise addition of aqueous HCl (1.0 N, 12 mk) at <10° C. The resulting yellow solid was isolated by filtration and dried under vacuum to give the crude compound which was purified by silica gel column (petroleum ether/ethyl acetate=5/1) to give 4-(2-bromo-6-hydroxy-phenyl)but-3-en-2-one (0.85 g, 3.53 mmol, 71% yield) as white solid, 1H NMR (400 MHz, DMSO-d6) δ ppm 10.85-10.93 (m, 1H) 7.66-7.78 (m, 1H) 7.09-7.21 (m, 3H) 6.92-7.00 (m, 1H) 2.28-2.35 (m, 3H).

### Step 2: 3-bromo-2-(3-hydroxybut-1-enyl)phenol

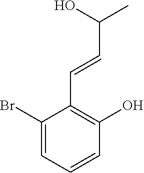

To a solution of 4-(2-bromo-6-hydroxy-phenyl)but-3-en-2-one (700 mg, 2.9 mmol) in tetrahydrofuran (10 ml) was slowly added sodium borohydride (121 mg, 3.2 mmol) at 0° C. and the mixture was stirred for 2 h at 25° C. The reaction was poured into HCl (1M in water, 5 ml) slowly at 0° C. and stirred for 10 min and then adjust pH>=7 by NaHCO₃(aq.). The mixture was taken up in ethyl acetate (20 ml) and the organic layers were washed with water (20 ml) and saturated brine solution (10 ml). The combined organic layers were dried (Na2SO4), concentrated to dryness in vacuo and purified by column chromatography (silica gel, petroleum ether/ethyl acetate=3/1) to give 3-bromo-2-(3-hydroxybut-1-enyl)phenol (590 mg, 2.43 mmol, 83.6% yield) as light yellow gum.

### Step 3: 3-bromo-2-(3-hydroxybutyl)phenol

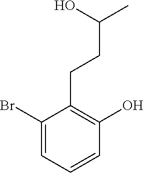

To a solution of 3-bromo-2-(3-hydroxybut-1-enyl)phenol (5.0 g, 20.57 mmol) in tetrahydrofuran (100 ml) was added tosylhydrazide (15.3 g, 82.3 mmol) and sodium acetate trihydrate (11.2 g, 82.3 mmol) and then stirred for 16 h at 70° C. The reaction was taken up in ethyl acetate (60 ml) and the organics washed with water (30 ml) and saturated brine solution (20 ml). The organics were separated and dried (Na2SO4) before concentration to dryness. The crude was purified by silica gel column (petroleum ether/ethyl acetate=3/1) to give 3-bromo-2-(3-hydroxybutyl)phenol (3.4 g, 13.87 mmol, 67.4% yield) as colorless oil. 1H NMR (400 MHz, DMSO-d6) δ ppm 9.71-9.78 (m, 1H) 6.88-7.06 (m, 2H) 6.74-6.84 (m, 1H) 4.43-4.54 (m, 1H) 3.96-4.09 (m, 1H) 3.57-3.70 (m, 1H) 2.72-2.86 (m, 1H) 2.56-2.68 (m, 1H) 1.38-1.62 (m, 2H) 1.08-1.13 (m, 2H).

### Step 4: 5-bromo-2-methyl-chromane

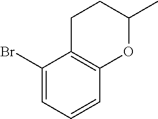

To a solution of 3-bromo-2-(3-hydroxybutyl)phenol (3.4 g, 13.0 mmol) in tetrahydrofuran (650 ml) was added diisopropyl azodicarboxylate (DIAD, 3.87 ml, 19.53 mmol) and triphenylphosphine (5.12 g, 19.53 mmol) at 0° C. and the mixture was stirred for 2 h at 25° C. The reaction was concentrated to dryness and the residue was taken up in ethyl acetate (50 ml). The combined organic layers were washed with water (30 ml) and saturated brine solution (10 ml). The organics were then separated and dried (Na2SO4) before being concentrated to dryness. The crude was then purified by silica gel column (petroleum ether/ethyl acetate=10/1) to give 5-bromo-2-methyl-chromane (1.9 g, 7.81 mmol, 60% yield) as colorless oil. 1H NMR (400 MHz, DMSO-d6) δ ppm 7.12 (dd, J=7.95, 0.98 Hz, 1H) 6.98-7.06 (m, 1H) 6.73-6.80 (m, 1H) 4.02-4.16 (m, 1H) 2.56-2.76 (m, 2H) 1.96-2.09 (m, 1H) 1.51-1.70 (m, 1H) 1.29-1.34 (m, 3H).

### Step 5: 5-benzylsulfanyl-2-methyl-chromane

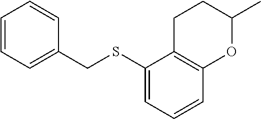 [SMILES: CC1CCc2c(cccc2SCc2ccccc2)O1]

To a solution of 5-bromo-2-methyl-chromane (1.10 g, 4.84 mmol) in tetrahydrofuran (30 ml) was added n-butyllithium solution (2.91 ml, 7.27 mmol) at −78° C. The mixture was then stirred at −78° C. for 0.5 h. A solution of dibenzyl disulfide (1.43 g, 5.8 mmol) in tetrahydrofuran (10 ml) was added at −78° C. The mixture was then stirred at −78° C. for another 1 h. The reaction mixture was quenched by adding saturated ammonium chloride solution (10 ml) and extracted with ethyl acetate (20 ml×3). The combined organic layers were washed with brine, dried over anhydrous sodium sulfate and concentrated under reduced pressure. The residue was purified by flash silica gel chromatography (silica gel, petroleum ether) to give 5-benzylsulfanyl-2-methyl-chromane (0.3 g, 1.11 mmol, 23% yield) as colorless oil. 1H NMR (400 MHz, DMSO-d6) δ ppm 1.31 (d, J=6.24 Hz, 3H) 1.58 (dtd, J=13.45, 10.79, 10.79, 5.56 Hz, 1H) 1.92-2.00 (m, 1H) 2.62-2.73 (m, 1H) 2.74-2.87 (m, 1H) 3.74 (s, 2H) 4.06-4.16 (m, 1H) 6.70 (d, J=8.19 Hz, 1H) 6.74-6.83 (m, 1H) 7.00-7.08 (m, 2H) 7.24-7.37 (m, 5H)

### Step 6: 2-methylchromane-5-sulfonyl chloride

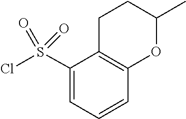

To a solution of 5-benzylsulfanyl-2-methyl-chromane (60 mg, 0.22 mmol, 1.0 eq) in acetic acid (1.8 ml) and water (0.2 ml) was added N-chlorosuccinimide (89 mg, 0.67 mmol, 3.0 eq) in three portions at 30° C. and the mixture was stirred for 16 h. The reaction was taken up in ethyl acetate (4 ml) and the combined organic layers were washed with water (2 ml) and saturated brine solution (2 ml). The combined organic layers were separated and dried (Na2SO4) before being concentrated to dryness. The crude was purified by preparative TLC (petroleum ether/ethyl acetate=20/1) to give 2-methylchromane-5-sulfonyl chloride (30 mg, 0.12 mmol, 55% yield) as colorless oil. For analysis an aliquot of this compound was reacted with aqueous ammonia and an LCMS sample of that reaction was measured: in accordance to the expected result MS m/z 226 ([M-Cl+NH2]⁻) was observed.

### Intermediate A7: 2-chloro-1,7-naphthyridine-5-sulfonyl chloride

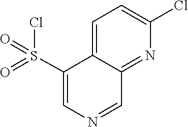 [SMILES: O=S(=O)(Cl)c1cncc2nc(Cl)ccc12]

### Step 1: 5-bromo-1-oxido-1,7-naphthyridin-1-ium

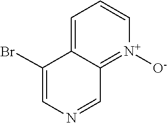 [SMILES: [O-][n+]1cccc2c(Br)cncc21]

In a 25 ml round bottom flask, 5-bromo-1,7-naphthyridine (2.5 g, 12.0 mmol) was dissolved in dichloromethane (17.5 ml) and cooled to 0-3° C. Next, m-chloroperoxybenzoic acid (3.1 g, 12.56 mmol) was added in portions over 20 min at 2-4° C. The reaction mixture was stirred at 2-22° C. for 5 h, then the solvent was removed under vacuo. The residue was purified over 80 g silica column with methanol in dichloromethane (0 to 10% in 30 min) to get a mixture of both possible N-oxides. This mixture was purified further using SFC (column chiral AY-H, 5 μm, 250×20 mm, 0-40% MeOH) to afford 5-bromo-1-oxido-1,7-naphthyridin-1-ium (394 mg, 14%) as yellow solid. MS m/z: 225.0 [M+H]+. ESI pos.

### Step 2: 5-benzylsulfanyl-1-oxido-1,7-naphthyridin-1-ium

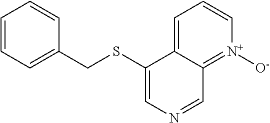 [SMILES: [O-][n+]1cccc2c(SCc3ccccc3)cncc21]

A mixture of 5-bromo-1-oxido-1,7-naphthyridin-1-ium (392 mg, 1.71 mmol), benzyl mercaptan (254 mg, 242 ul, 2.05 mmol), tris(dibenzylideneacetone)dipalladium(0) (46.9 mg, 0.051 mmol), (9,9-dimethyl-9H-xanthene-4,5-diyl)bis(diphenylphosphine) (49 mg, 0.085 mmol), N,N-diisopropyl ethylamine (441 mg, 600 ul, 3.41 mmol) in 1,4-dioxane (12 ml) was heated at 90° C. for 5 h. After cooling down to room temperature, the mixture was concentrated under reduced pressure and purified by flash chromatography (25 g SiO2, gradient 0 to 20% methanol in dichloromethane) to afford 5-(benzylthio)-1-oxido-1,7-naphthyridin-1-ium (440 mg, 90%) as orange solid. MS m/z 269.1 [M+H]+, ESI pos.

### Step 3: 5-benzylsulfanyl-2-chloro-1,7-naphthyridine

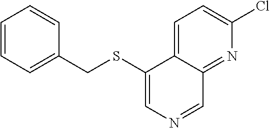 [SMILES: Clc1ccc2c(SCc3ccccc3)cncc2n1]

To a stirred solution of 5-(benzylthio)-1-oxido-1,7-naphthyridin-1-ium (159 mg, 0.557 mmol) in dichloromethane (extra dry, 5.5 ml) at 0° C. was added phosphorus oxychloride (102 mg, 62 ul, 0.668 mmol) followed by dropwise addition of dimethylformamide (20 mg, 22 ul, 0.28 mmol) under argon. The resulting reaction mixture was warmed to 23° C. and stirred at this temperature for 6 h. Saturated aqueous sodium carbonate solution was added to the reaction mixture slowly to adjust the pH to 7-8. The resulting mixture was separated and the aqueous phase was extracted with dichloromethane thoroughly. The combined organic layers were washed with brine, dried over Na2SO4, filtered, concentrated under reduced pressure and purified by flash chromatography column (silica gel, 10 to 70% ethyl acetate in heptane) to afford 5-(benzylthio)-2-chloro-1,7-naphthyridine (50 mg, 31%) as light yellow solid, MS m/z 287.1 [M+H]+, ESI pos

### Step 4: 2-chloro-1,7-naphthyridine-5-sulfonyl chloride

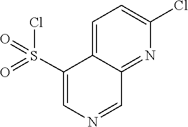 [SMILES: O=S(=O)(Cl)c1cncc2nc(Cl)ccc12]

5-(Benzylthio)-2-chloro-1,7-naphthyridine (45 mg, 0.155 mmol) was dissolved in a mixture of acetic acid (390 ul) and water (39 ul). N-Chlorosuccinimide (62 mg, 0.466 mmol) was added and the mixture was stirred at 23° C. for 1.5 h. Dichloromethane was added and the mixture was stirred at room temperature for 5 min. The layers were separated and the aqueous layer was extracted with dichloromethane. The combined organic layers were dried over Na2SO4, filtered and concentrated under reduced pressure to afford 2-chloro-1,7-naphthyridine-5-sulfonyl chloride (47 mg, 34.5%) as yellow oil which was used crude directly in the next step without further purification MS m/z 263.0 [M+H]+, ESI pos.

### Intermediate A8: 2-methylquinoline-5-sulfonyl chloride

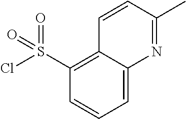

Chlorosulfonic acid (5.34 g, 3.07 ml, 45.4 mmol) was added carefully to quinaldine (500 mg, 472 ul, 3.49 mmol) and the reaction mixture was heated to 140° C. for 2 h. LCMS showed complete conversion and formation of 3 regioisomers. The reaction mixture was cooled to room temperature and carefully quenched with ice/ethyl acetate. The organic layer was separated and the aqueous layer was extracted with ethyl acetate. The combined organic layers were dried over Na2SO4, filtered and concentrated in vacuo. The crude material was purified by flash chromatography on silica gel (80 g, 0% to 100% ethyl acetate in heptane) to afford 2-methylquinoline-8-sulfonyl chloride (255.8 mg, 29.4%) as white solid, 2-methylquinoline-6-sulfonyl chloride (25 mg, 3.0%) as light brown solid and the title compound 2-methylquinoline-5-sulfonyl chloride (21.9 mg, 2.4%) as white solid. 1H NMR (600 MHz, DMSO-d6) δ ppm 9.69 (d, J=9.1 Hz, 1H), 8.20 (dt, J=8.5, 1.0 Hz, 1H), 8.18 (dd, J=7.3, 1.1 Hz, 1H), 8.14-8.15 (m, 1H), 8.01-8.07 (m, 2H), 2.95 (s, 3H).

### Intermediate A9: 6-chloronaphthalene-1-sulfonyl chloride

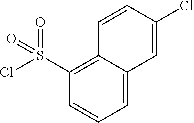 [SMILES: O=S(=O)(Cl)c1cccc2cc(Cl)ccc12]

### Step 1: 6-chloro-1-iodo-naphthalene

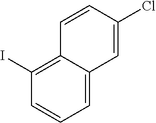 [SMILES: Clc1ccc2c(I)cccc2c1]

To a solution of p-toluenesulfonic acid monohydrate (803.16 mg, 4.22 mmol) in acetonitrile (5 ml) was added (6-chloro-1-naphthyl)amine (CAS 50885-10-4, 250 mg, 1.41 mmol). The resulting suspension of amine salt was cooled to 0° C., and a solution of sodium nitrite (194 mg, 2.81 mmol) and potassium iodide (584 mg, 3.52 mmol) in water (0.85 ml) was added gradually. The reaction mixture was stirred for 30 min at 0° C., then allowed to come to room temperature and stirred for additional 2 h. The reaction mixture was diluted with saturated NaHCO₃ solution and extracted two times with ethyl acetate. The organic layers were washed with water and brine, dried over Na2SO4 and concentrated to dryness. The crude material was purified by flash chromatography on silica gel (40 g, ethyl acetate in heptane 0-30%) to afford 6-chloro-1-iodo-naphthalene (20 mg, 5% yield) as yellow oil. 1H NMR (300 MHz, DMSO-d6) δ=8.17 (dd, J=1.0, 7.5 Hz, 1H), 8.11 (d, J=2.2 Hz, 1H), 8.05-7.97 (m, 2H), 7.67 (dd, J=2.2, 8.9 Hz, 1H), 7.34 (dd, J=7.5, 8.3 Hz, 1H).

### Step 2: 1-benzylsulfanyl-6-chloro-naphthalene

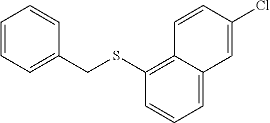 [SMILES: Clc1ccc2c(SCc3ccccc3)cccc2c1]

6-Chloro-1-iodo-naphthalene (18 mg, 0.062 mmol) was dissolved in 1,4-dioxane (extra dry, (0.6 ml), then benzyl mercaptan (8.52 mg, 8 ul, 0.069 mmol), tris(dibenzylidene-acetone)dipalladium(0) (1.7 mg, 0.002 mmol), Xantphos (1.8 mg, 0.003 mmol) and N.N-diisopropyl ethylamine (16 mg, 22 ul, 0.125 mmol) were added at room temperature under argon. The mixture was stirred at 90° C. for 3 hours. The reaction mixture was diluted with saturated NaHCO₃-solution and extracted two times with ethyl acetate. The organic layers were washed with water and brine, dried over Na2SO4 and concentrated to dryness. The crude material was purified by flash chromatography on silica gel (12 g, ethyl acetate in heptane 0-30%) to afford 1-benzylsulfanyl-6-chloro-naphthalene (16 mg, 86%) as light yellow oil.

### Step 3: 6-chloronaphthalene-1-sulfonyl chloride

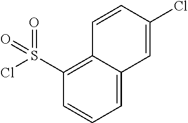 [SMILES: O=S(=O)(Cl)c1cccc2cc(Cl)ccc12]

1-benzylsulfanyl-6-chloro-naphthalene (16 mg, 0.054 mmol) was dissolved in acetic acid (0.5 ml) and water (0.05 ml). N-chlorosuccinimide (22 mg, 0.162 mmol) was added and the mixture was stirred at room temperature for 1 h. Dichloromethane and a few drops of water were added to the mixture. After stirring it at room temperature for 5 min the layers were separated and the aqueous layer was extracted with dichloromethane. The combined organic layers were dried over Na2SO4, filtered and concentrated under reduced pressure to obtain a yellow oil. The crude was used directly to the next step without further purification.

### Intermediate A10: 5-chloronaphthalene-1-sulfonyl chloride

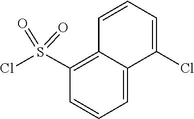 [SMILES: O=S(=O)(Cl)c1cccc2c(Cl)cccc12]

Intermediate A10 is commercial (CAS: 6291-07-2)

### Intermediate A11: 7-chloronaphthalene-1-sulfonyl chloride

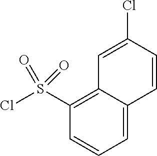 [SMILES: O=S(=O)(Cl)c1cccc2ccc(Cl)cc12]

Intermediate A11 is commercial (CAS: 102153-62-8)

### Intermediate A12: 8-chloro-1-hydroxy-isoquinoline-4-sulfonyl chloride

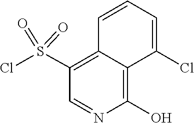 [SMILES: O=S(=O)(Cl)c1cnc(O)c2c(Cl)cccc12]

### Step 1: 8-chloro-2-oxido-isoquinolin-2-ium

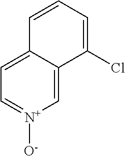 [SMILES: [O-][n+]1ccc2cccc(Cl)c2c1]

To the stirred solution of 8-chloroisoquinoline (10.0 g, 61.12 mmol, 1.0 eq) in dichloromethane (250 ml) was added portion wise 3-chloroperoxybenzoic acid (25.6 g, 103.9 mmol) and the reaction mixture was stirred at 25° C. for 3 h. Next, dichloromethane (300 ml) and methanol (50 ml) was added. The organic layer was washed with 2M sodium hydroxide solution (2×300 ml), the organic layer was dried (Na2SO4) and evaporated to dryness to give 8-chloro-2-oxido-isoquinolin-2-ium (10.0 g, 55.68 mmol, 77.4% yield). 1H NMR (500 MHz, CDCl3) δ=9.11 (s, 1H), 8.14 (dd, J=1.6, 7.1 Hz, 1H), 7.72-7.60 (m, 3H), 7.51-7.44 (m, 1H)

### Step 2: 8-chloroisoquinolin-1-ol

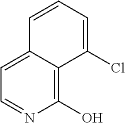 [SMILES: Oc1nccc2cccc(Cl)c12]

8-Chloro-2-oxido-isoquinolin-2-ium (10.0 g, 55.7 mmol) was stirred in acetic anhydride (250 ml) at 140° C. for 16 h. The reaction mixture was evaporated to dryness, then 350 ml of a 2M aqueous sodium hydroxide solution was added to the oily residue, and the reaction mixture was stirred at 80° C. for 3 h. The mixture was allowed to cool down and was acidified to pH 4-5 (4M aq. NaHSO4 solution) and extracted with dichloromethane (2×300 ml). The combined organic layers were dried (Na2SO4) and evaporated to give 8-chloroisoquinolin-1-ol as a brown solid (11.0 g, 61.25 mmol, 82.5% yield). 1H NMR (500 MHz, DMSO-d6) δ=11.22 (br s, 7H), 7.59-7.50 (m, 3H), 7.50-7.36 (m, 2H), 7.20-7.11 (m, 1H), 6.50 (d, J=7.1 Hz, 1H)

### Step 3: 8-chloro-1-hydroxy-isoquinoline-4-sulfonyl chloride

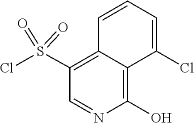 [SMILES: O=S(=O)(Cl)c1cnc(O)c2c(Cl)cccc12]

To 8-chloroisoquinolin-1-ol (3.0 g, 16.7 mmol, 1.0 eq) was added chlorosulfonic acid (11.1 ml, 167 mmol) at 100° C. and the reaction mixture was left at 100° C. for 16 h. The reaction mixture was poured very carefully onto ice (200 g) and quickly extracted with ethyl acetate (300 ml). The organic layer was dried and evaporated to dryness, the residue was triturated in dichloromethane (5×30 ml), filtered under argon atmosphere and dried to give 8-chloro-1-hydroxy-isoquinoline-4-sulfonyl chloride (925 mg, 3.33 mmol, 19% yield. 1H NMR (500 MHz, CDCl3) δ=10.87 (br, s, 1H), 9.03-9.07 (m, 1H), 8.97-9.02 (m, 1H), 8.63 (q, J=7.8 Hz, 1H), 8.56-8.48 (m, 1H).

### Intermediate A13: 7-chloro-1-oxo-2H-isoquinoline-4-sulfonyl chloride

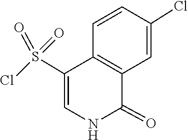 [SMILES: O=c1[nH]cc(S(=O)(=O)Cl)c2ccc(Cl)cc12]

A solution of 7-chloroisoquinoline-1(2H)-one (CAS 24188-74-7, 14.5 mg, 0.081 mmol) and chlorosulfonic acid (261 mg, 150 ul, 2.17 mmol) was stirred at 95° C. for 2 hr. The reaction mixture was treated with water (5 ml) and extracted with ethyl acetate (3×5 ml). The combined organic layers were washed with water, brine, dried over sodium sulfate, filtered and concentrated to dryness to afford 7-7-chloro-1-oxo-2H-isoquinoline-4-sulfonyl chloride (22.5 mg, 95%) as light yellow solid. MS 278.1 [M+H]+, ESI pos., 1H NMR (300 MHz, CDCl3) δ ppm 7.84-7.90 (m, 1H) 8.15-8.20 (m, 1H) 8.30-8.35 (m, 1H) 8.45-8.48 (m, 1H) 9.67-9.85 (m, 1H).

### Intermediate A14: 2-chloro-8-oxo-7H-1,7-naphthyridine-5-sulfonyl chloride

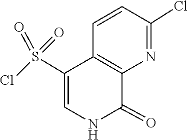 [SMILES: O=c1[nH]cc(S(=O)(=O)Cl)c2ccc(Cl)nc12]

### Step 1: 1-oxido-7H-1,7-naphthyridin-1-ium-8-one

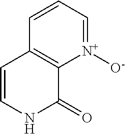 [SMILES: O=c1[nH]ccc2ccc[n+]([O-])c12]

To the stirred solution of 1,7-naphthyridin-8-ol (CAS 67967-11-7, 0.5 g, 3.42 mmol) in acetic acid (15 ml) was added portionwise hydrogen peroxide (30% in water, 0.78 ml, 6.84 mmol) and the reaction mixture was stirred at 80° C. for 16 h. The reaction mixture was carefully evaporated to dryness to give 600 mg of a dark brown solid which was used in the next step without further purification.

### Step 2: 2-chloro-7H-1,7-naphthyridin-8-one

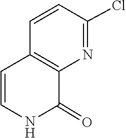 [SMILES: O=c1[nH]ccc2ccc(Cl)nc12]

To a suspension of 1-oxido-7H-1,7-naphthyridin-1-ium-8-one (600 mg, 3.7 mmol) in dimethylformamide (15 ml) was added dropwise oxalyl chloride (0.44 ml, 5.18 mmol) at 0° C. After complete addition, the mixture was allowed to warm to room temperature and stirred for 30 min. Methanol was added (20 ml) to quench excess oxalyl chloride, and the mixture was evaporated to dryness. The residue was purified by chromatography (silica gel, ethyl acetate in heptane 0-50%) to give 2-chloro-7H-1,7-naphthyridin-8-one (200 mg, 1.11 mmol, 28% yield). 1H NMR (500 MHz, DMSO-d6) δ=7.90 (d, J=8.4 Hz, 1H), 7.59 (d, J=8.4 Hz, 1H), 7.43-7.34 (m, 1H), 7.29-7.22 (m, 1H), 6.54 (br d, J=6.4 Hz, 1H).

### Step 3: 2-chloro-8-oxo-7H-1,7-naphthyridine-5-sulfonyl chloride

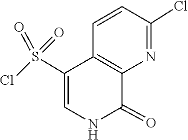 [SMILES: O=c1[nH]cc(S(=O)(=O)Cl)c2ccc(Cl)nc12]

To 2-chloro-7H-1,7-naphthyridin-8-one (200 mg, 1.11 mmol) was added chlorosulfonic acid (0.74 ml, 11.1 mmol) at 90° C. and the reaction mixture was left at 90° C. for 16 h. To the reaction mixture was added ice water (10 ml), then it was extracted with ethyl acetate (50 ml). The organic layer was dried and evaporated to give 2-chloro-8-oxo-7H-1,7-naphthyridine-5-sulfonyl chloride as a brown solid (91.8 mg, 0.33 mmol, 28.2% yield), 1H NMR (400 MHz, DMSO-d6) δ=11.60 (br, s, 1H), 8.77 (br d, J=8.7 Hz, 1H), 7.83 (br d, J=8.8 Hz, 1H), 7.52 (br s, 1H)

### Intermediate A15: 7-chloro-2-oxo-1H-quinoline-4-sulfonyl chloride

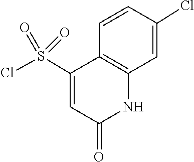 [SMILES: O=c1cc(S(=O)(=O)Cl)c2ccc(Cl)cc2[nH]1]

The title compound was prepared in analogy to Intermediate A4 from 4,7-dichloro-2-(1H)-quinolinone (CAS 156820-88-1) instead of 4-bromo-7-chloro-isoquinoline as a light yellow solid. MS (ESI) m/z: 278.0 [M+H]+.

### Intermediate A16: 7-chloro-1-(methylamino)isoquinoline-4-sulfonyl chloride

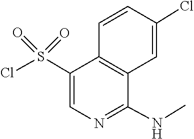 [SMILES: CNc1ncc(S(=O)(=O)Cl)c2ccc(Cl)cc12]

### Step 1: 7-chloro-N-methyl-isoquinolin-1-amine

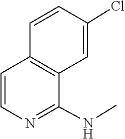

A mixture of 1,7-dichloroisoquinoline (CAS 70810-24-1, 298 mg, 1.46 mmol), N,N-diisopropyl ethylamine (377 mg, 510 ul, 2.92 mmol) and methylamine (2M in THF, 2.19 ml, 4.38 mmol) in tetrahydrofuran (4 ml) was stirred 2 h at 60° C. for 72 h. The reaction mixture was poured into water and extracted with ethyl acetate twice. The combined organic layers were washed with brine, dried over Na2SO4, filtered and concentrated in vacuo. The crude material was purified by flash chromatography (silica gel, 12 g, 0% to 50% ethyl acetate in heptane) to yield a light yellow solid (127 mg, 43% yield), MS m/z 193.1 [M+H]+, ESI pos.

### Step 2: 4-bromo-7-chloro-N-methyl-isoquinolin-1-amine

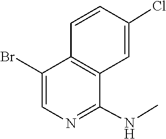

A mixture of 7-chloro-N-methyl-isoquinolin-1-amine (125 mg, 0.649 mmol) and N-bromosuccinimide (115.49 mg, 0.649 mmol) in acetonitrile (3 ml) was stirred 1 h at room temperature. The reaction mixture was poured into water and extracted with ethyl acetate twice. The combined organic layers were washed with brine, dried over Na2SO4, filtered and concentrated in vacuo. The crude material was purified by flash chromatography (silica gel, 25 g, 0% to 50% ethyl acetate in heptane) to yield) an off-white solid yellow oil (134 mg, 73% yield). MS 273.1 [M+H]+, ESI pos.

### Step 3: 4-benzylsulfanyl-7-chloro-N-methyl-isoquinolin-1-amine

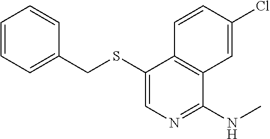 [SMILES: CNc1ncc(SCc2ccccc2)c2ccc(Cl)cc12]

A mixture of 4-bromo-7-chloro-N-methyl-isoquinolin-1-amine (133 mg, 0.490 mmol), Xantphos (14.17 mg, 0.024 mmol), Pd2(dba)3 (13.46 mg, 0.015 mmol), N,N-diisopropyl ethylamine (126.6 mg, 171 ul, 0.98 mmol) and benzyl mercaptan (67 mg, 63.7 ul, 0.539 mmol) in 1,4-dioxane (3 ml) was heated to 90° C. and stirred for 4 h. The reaction mixture was poured into water and extracted with ethyl acetate twice. The organic layers were combined, dried over Na2SO4 and concentrated in vacuo. The crude material was purified by flash chromatography (silica gel, 12 g, 0% to 50% ethyl acetate in heptane) to afford a yellow oil (140 mg, 86% yield). MS m/z 315.1 [M+H]+, ESI pos.

### Step 4: 7-chloro-1-(methylamino)isoquinoline-4-sulfonyl chloride

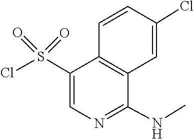 [SMILES: CNc1ncc(S(=O)(=O)Cl)c2ccc(Cl)cc12]

To a solution of 4-benzylsulfanyl-7-chloro-N-methyl-isoquinolin-1-amine (133 mg, 0.422 mmol) in acetic acid (2 ml) and water (0.2 ml) was added N-chlorosuccinimide (169 mg, 1.27 mmol) at room temperature. The clear solution was stirred for 3 h at room temperature. The reaction was extracted with ice water and twice with dichloromethane. The combined organic layers were dried with MgSO4, filtered and concentrated in vacuo to afford a yellow oil (120 mg, 70% purity, 97% yield). MS 291.0 [M+H]+, ESI pos. The crude material was directly used for the next step.

### Intermediate A17: 7-chloro-2-(methylamino)quinoline-4-sulfonyl chloride

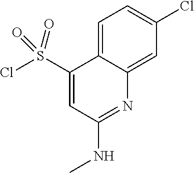 [SMILES: CNc1cc(S(=O)(=O)Cl)c2ccc(Cl)cc2n1]

### Step 1: 4-benzylsulfanyl-7-chloro-1H-quinolin-2-one

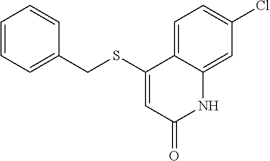 [SMILES: O=c1cc(SCc2ccccc2)c2ccc(Cl)cc2[nH]1]

A mixture of 4,7-dichloro-1H-quinolin-2-one (CAS 156820-88-1, see patent WO2004/2960, 458 mg, 2.14 mmol), Xantphos (61.9 mg, 0.107 mmol), Pd2(dba)3 (58.78 mg, 0.064 mmol), N,N-diisopropyl ethylamine (553 mg, 748 ul, 4.28 mmol) and benzyl mercaptan (292 mg, 278 μL, 2.35 mmol) in 1,4-dioxane (4 ml) was heated to 100° C. and stirred for 2 h to yield an off-white suspension The reaction mixture was poured into water and extracted with ethyl acetate twice. The organic layers were combined, dried over Na2SO4 and concentrated in vacuo. The residue was triturated with dichloromethane and filtrated. The mother liquor was concentrated and the residue was purified by flash chromatography (silica gel, 50 to 100% ethyl acetate in heptane) and the product was combined with the filter cake to afford an off-white solid (522 mg, 81%). MS m/z: 302.1 [M+H]+, ESI pos.

### Step 2: 4-benzylsulfanyl-2,7-dichloro-quinoline

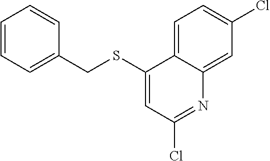 [SMILES: Clc1ccc2c(SCc3ccccc3)cc(Cl)nc2c1]

A mixture of 4-benzylsulfanyl-7-chloro-1H-quinolin-2-one (549 mg, 1.73 mmol) and phosphorus oxychloride (2.38 g, 1.45 ml, 15.55 mmol) was stirred 1 h at 100° C. The excess of phosphorus oxychloride was removed in vacuo. The reaction mixture was diluted with dichloromethane and poured into saturated aqueous NaHCO₃ solution containing ice. The mixture was stirred at room temperature for 20 min and extracted twice with dichloromethane. The combined organic layers were dried over Na2SO4, filtered and the residual solvent was removed under reduced pressure. The crude material was purified by flash chromatography (silica gel, 0% to 50% ethyl acetate in heptane) to afford an off-white solid (454 mg, 92% yield). MS m/z: 320.0 [M+H]+, ESI pos.

### Step 3: 4-benzylsulfanyl-7-chloro-N-methyl-quinolin-2-amine

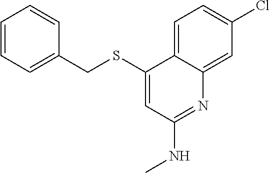 [SMILES: CNc1cc(SCc2ccccc2)c2ccc(Cl)cc2n1]

A mixture of 4-benzylsulfanyl-2,7-dichloro-quinoline (250 mg, 0.781 mmol), N,N-diisopropyl ethylamine (303 mg, 409 ul, 2.34 mmol) and methylamine (2M in THF, 3 ml, 6 mmol) in tetrahydrofuran (1 ml) was stirred at 60° C. for 3 days. The reaction mixture was poured into water and extracted with ethyl acetate twice. The combined organic layers were washed with brine, dried over Na2SO4, filtered and concentrated in vacuo. The crude material was purified by flash chromatography (silica gelz, 0% to 50% ethyl acetate in heptane) to yield a white solid (180 mg, 73% yield). MS m/z 315.1 [M+H]+, ESI pos.

### Step 4: 7-chloro-2-(methylamino)quinoline-4-sulfonyl chloride

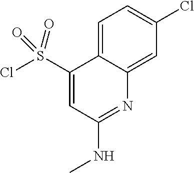 [SMILES: CNc1cc(S(=O)(=O)Cl)c2ccc(Cl)cc2n1]

To a solution of 4-benzylsulfanyl-7-chloro-N-methyl-quinolin-2-amine (177 mg, 0.562 mmol) in acetic acid (2 ml) and water (0.2 ml) was added N-chlorosuccinimide (225 mg, 1.69 mmol) at room temperature. The clear solution was stirred for 3 h at room temperature, then water was added and the mixture was extracted with ethyl acetate twice. The combined organic layers were dried with Na2SO4, filtered, concentrated in vacuo and used directly in the next step. Yellow amorphous solid. MS m/z: 291.1 [M+H]+, ESI pos.

### Intermediate A18: 2-(dimethylamino)quinoline-5-sulfonyl chloride

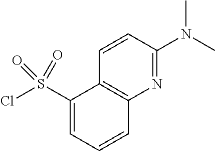 [SMILES: CN(C)c1ccc2c(S(=O)(=O)Cl)cccc2n1]

### Step 1: 5-bromo-N.N-dimethyl-quinolin-2-amine

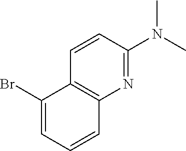

In a sealed glass tube, 5-bromo-2-chloro-quinoline (CAS 99455-13-7, 250 mg, 1.03 mmol) was dissolved in tetrahydrofuran (extra dry, 2 ml), then dimethylamine (2M in THF, 1.84 g, 2.06 ml, 4.12 mmol) and diisopropyl ethylamine (266 mg, 360 μL, 2.06 mmol) was added. The reaction mixture was stirred for 24 h at 60° C. At room temperature ethyl acetate and water was added. The organic layer was washed with saturated NaCl-solution, dried over MgSO4 and concentrated in vacuo to get 5-bromo-N,N-dimethyl-quinolin-2-amine (260 mg, 98%) as light yellow solid. MS (ESI) m/z 251.1 [M+H]+.

### Step 2: 5-benzylsulfanyl-N,N-dimethyl-quinolin-2-amine

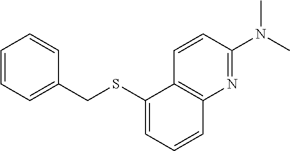 [SMILES: CN(C)c1ccc2c(SCc3ccccc3)cccc2n1]

A mixture of 5-bromo-N,N-dimethyl-quinolin-2-amine (260 mg, 1.04 mmol), benzyl mercaptan (142 mg, 135 ul, 1.14 mmol), N,N-diisopropyl ethylamine (268 mg, 362 ul, 2.07 mmol), 9,9-dimethyl-4,5-bis(diphenylphosphino)xanthene (30 mg, 0.052 mmol) and tris(dibenzylideneacetone)dipalladium(0) (28 mg, 0.031 mmol) in 1,4-dioxane (3 ml) was heated to 95° C. and stirred for 16 h. The reaction mixture was filtered and washed with ethyl acetate twice. The combined organic layers were dried (Na2SO4) and concentrated in vacuo. The crude material was purified by flash chromatography (silica gel, 0% to 80% ethyl acetate in n-heptane) to afford an orange solid (204 mg, 67%). MS (ESI) m/z: 295.2 [M+H]+.

### Step 3: 2-(dimethylamino)quinoline-5-sulfonyl chloride

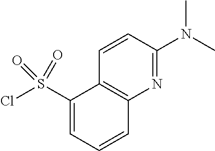 [SMILES: CN(C)c1ccc2c(S(=O)(=O)Cl)cccc2n1]

In a 20 ml glass tube, 5-benzylsulfanyl-N,N-dimethyl-quinolin-2-amine (66 mg, 0.224 mmol) was dissolved in acetic acid (0.9 ml) and water (0.1 ml). At room temperature N-chlorosuccinimide (90 mg, 0.673 mmol) was added in portions over 15 min. The reaction mixture was stirred 1 h at room temperature. The solvent was removed in vacuo. The residue was extracted with ethyl acetate twice. The combined organic layers were washed with water, saturated NaCl solution, dried over MgSO4, filtered and the solvent was removed in vacuo to afford 2-(dimethylamino)quinoline-5-sulfonyl chloride (65 mg, 96%) as yellow solid which was used in next step without further purification. MS (ESI) m/z 271.1[M+H]⁺

### Intermediate A19: 7-chloro-1-methoxy-isoquinoline-4-sulfonyl chloride

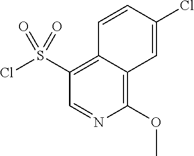 [SMILES: COc1ncc(S(=O)(=O)Cl)c2ccc(Cl)cc12]

### Step 1: 4-bromo-7-chloro-1-methoxy-isoquinoline

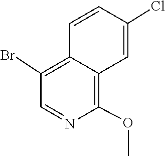

To a solution of freshly prepared sodium methylate (2.93 g, 54.2 mmol, 3.0 eq) in methanol (100 ml), 4-bromo-1,7-dichloro-isoquinoline (CAS 953421-74-4, 5.0 g, 18.05 mmol, 1.0 eq) was added under argon and the resulting mixture was refluxed overnight. The mixture was concentrated under reduced pressure and the residue was partitioned between water and dichloromethane. The combined organic layers were washed with brine, dried over sodium sulfate and concentrated under reduced pressure to give 4-bromo-7-chloro-1-methoxy-isoquinoline (4.3 g, 15.8 mmol, 87.5% yield) as brown powder. MS (ESI) m/z: 272.0/274.0/276.0 [M+H]+.

The following steps were performed in analogy to Intermediate A18 using 4-bromo-7-chloro-1-methoxy-isoquinoline instead of 5-bromo-N,N-dimethyl-quinolin-2-amine in step 2. The title compound was used without further purification in the next step.

### Intermediate A20: 7-chloro-8-cyano-isoquinoline-4-sulfonyl chloride

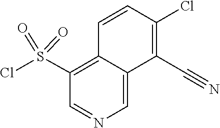 [SMILES: N#Cc1c(Cl)ccc2c(S(=O)(=O)Cl)cncc12]

### Step 1: 7-chloro-isoquinoline-8-carbonitrile

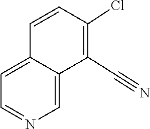 [SMILES: N#Cc1c(Cl)ccc2ccncc12]

8-Bromo-7-chloro-isoquinoline (CAS 2092424-29-6, 300 mg, 1.24 mmol) was dissolved in N,N-dimethylacetamide (3 ml) and under argon, dicyanozinc (73 mg, 0.619 mmol) and tetrakis(triphenylphosphine)palladium(0) (71.5 mg, 0.062 mmol) were added. The mixture was stirred over night at 100° C. The reaction mixture was diluted with water and extracted two times with ethyl acetate. The organic layers were washed with water and brine, dried over Na2SO4 and concentrated to dryness. The crude material was purified by flash chromatography (silica gel, 0-100% ethyl acetate in heptane) to afford a light-yellow solid (85 mg, 36% yield), MS m/z 189.0 [M+H]+, ESI pos.

### Step 2: 4-bromo-7-chloro-isoquinoline-8-carbonitrile

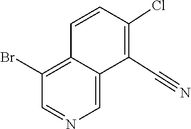 [SMILES: N#Cc1c(Cl)ccc2c(Br)cncc12]

7-chloroisoquinoline-8-carbonitrile (85 mg, 0.451 mmol) was dissolved in 1,2-dichloroethane (extra dry, 2 ml). Iodobenzene diacetate (218 mg, 0.676 mmol) and potassium bromide (268 mg, 2.25 mmol) were added and the mixture was stirred at 70° C. for 16 h. The mixture was extracted with water and dichloromethane and the combined organic layers were dried and concentrated in vacuo. The crude was purified by flash column (silica gel, 0-80% ethyl acetate in heptane) to afford a white solid (65 mg, 54% yield), MS m/z 269.0[M+H]+, ESI pos.

### Step 3: 4-benzylsulfanyl-7-chloro-isoquinoline-8-carbonitrile

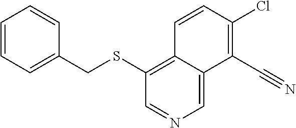 [SMILES: N#Cc1c(Cl)ccc2c(SCc3ccccc3)cncc12]

A mixture of 4-bromo-7-chloro-isoquinoline-8-carbonitrile (60 mg, 0.224 mmol), Xantphos (6.5 mg, 0.011 mmol), Pd2(dba)3 (6.2 mg, 0.0067 mmol), N,n-diisopropyl ethylamine (58 mg, 78 ul, 0.45 mmol) and benzyl mercaptan (30.6 mg, 29 ul, 1.37 mmol) in 1,4-dioxane (2 ml) was stirred for 1.5 h at 70° C. The reaction mixture was poured into water and extracted with ethyl acetate twice. The combined organic layers were dried over Na2SO4 and concentrated in vacuo. The crude material was purified by flash chromatography (silica gel, 0% to 100% ethyl acetate in heptane) to afford a yellow solid (28 mg, 40% yield), MS m/z 311.0 [M+H]+, ESI pos.

### Step 4: 7-chloro-8-cyano-isoquinoline-4-sulfonyl chloride

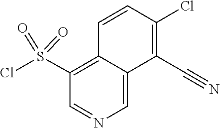 [SMILES: N#Cc1c(Cl)ccc2c(S(=O)(=O)Cl)cncc12]

4-Benzylsulfanyl-7-chloro-isoquinoline-8-carbonitrile (47 mg, 0.151 mmol) was dissolved in acetic acid (500 ul) and water (50 ul). N-chlorosuccinimide (60.6 mg, 0.454 mmol) was added and the mixture was stirred for 2 h at room temperature. The reaction mixture was diluted with water and extracted two times with dichloromethane. The combined organic layers were washed with water and brine, dried over Na2SO4 and concentrated in vacuo to afford a yellow solid which was used directly in the next step.

### Intermediate A21: 2-(difluoromethyl)quinoline-5-sulfonyl chloride

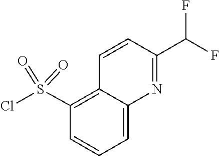 [SMILES: O=S(=O)(Cl)c1cccc2nc(C(F)F)ccc12]

2-(Difluoromethyl)quinoline (CAS 1075184-01-8, 150 mg, 0.837 mmol) was added slowly to chlorosulfonic acid (1.27 g, 723 μL, 10.88 mmol) at 0° C. The mixture was stirred at room temperature for 1 hr, then at 140° C. for 20 h. The reaction mixture was slowly added to an ice/ethyl acetate mixture under stirring, then extracted with ethyl acetate (3×), dried over Na2SO4, filtered and concentrated in vacuo. The crude light orange oil was purified by flash chromatography (silica gel, 0 to 100% ethyl acetate in n-heptane) to afford 2-(difluoromethyl)quinoline-5-sulfonyl chloride (9.3 mg, 3.2%) as white solid. ¹H NMR (600 MHz, DMSO-d6) δ ppm 9.39 (d, J=8.9 Hz, 1H), 8.70 (d, J=8.6 Hz, 1H), 8.31 (d, J=1.5 Hz, 1H), 8.01-8.09 (m, 2H), 7.85 (d, J=8.9 Hz, 1H), 7.76-7.83 (m, 1H), 6.99-7.22 (m, 1H).

### Intermediate A22: 5-(dimethylamino)naphthalene-1-sulfonyl chloride

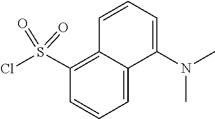 [SMILES: CN(C)c1cccc2c(S(=O)(=O)Cl)cccc12]

### Intermediate A22 is commercial (CAS: 605-65-2)

Intermediate A23: 7-chloro-2-methyl-1-oxo-isoquinoline-4-sulfonyl chloride

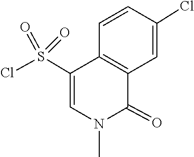 [SMILES: Cn1cc(S(=O)(=O)Cl)c2ccc(Cl)cc2c1=O]

### Step 1: 7-chloro-2-methyl-isoquinolin-1-one

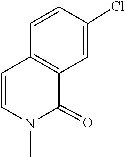

7-Chloroisoquinolin-1-ol (CAS 24188-74-7, 100 mg, 0.557 mmol, 1 eq) was dissolved in N,N-dimethylformamide (extra dry, 1 ml). Cesium carbonate (272 mg, 0.835 mmol) and iodomethane (395 mg, 174 ul, 2.78 mmol) were added and the mixture was stirred at 60° C. After 90 min the mixture was cooled to room temperature, quenched with saturated Na2CO3 solution and extracted with ethyl acetate. The combined organic layers were dried over Na2SO4 and concentrated to give a white solid (107 mg, 99% yield), 1H NMR (600 MHz, CDCl3) δ ppm 8.40 (d, J=2.3 Hz, 1H), 7.56 (dd, J=8.5, 2.3 Hz, 1H), 7.45 (d, J=8.5 Hz, 1H), 7.07 (d, J=7.4 Hz, 1H), 6.46 (d, J=7.2 Hz, 1H), 3.60 (s, 3H).

### Step 2: 7-chloro-2-methyl-1-oxo-isoquinoline-4-sulfonyl chloride

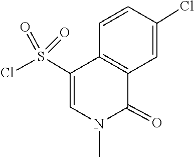 [SMILES: Cn1cc(S(=O)(=O)Cl)c2ccc(Cl)cc2c1=O]

7-Chloro-2-methyl-isoquinolin-1-one (70 mg, 0.343 mmol) was dissolved in chlorosulfonic acid (522 mg, 300 ul, 4.48 mmol) and the reaction mixture was heated at 100° C. for 1 h. The mixture was poured onto ice with ethyl acetate and extracted with ethyl acetate. The combined organic layers were dried over Na2SO4 and concentrated to afford a white solid (73 mg, 73% yield). The material was directly used for the next step.

### Intermediate A24: 7-chloro-1-methyl-2-oxo-quinoline-4-sulfonyl chloride

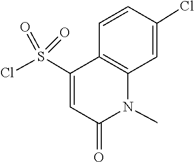 [SMILES: Cn1c(=O)cc(S(=O)(=O)Cl)c2ccc(Cl)cc21]

The title compound was prepared in analogy to Intermediate A4 from 4,7-dichloro-1-methyl-quinolin-2-one (CAS 98994-41-3) instead of 4-bromo-7-chloro-isoquinoline as a yellow solid. MS (ESI) m/z: 292.0 [M+H]+.

### Intermediate A25: 7,8-dichloro-1-oxo-2H-isoquinoline-4-sulfonyl chloride

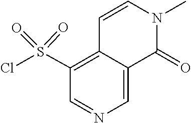 [SMILES: Cn1ccc2c(S(=O)(=O)Cl)cncc2c1=O]

The title compound was prepared in analogy to Intermediate A13 from 7,8-dichloro-2H-isoquinolin-1-one (CAS 80233-87-0) instead of 7-chloroisoquinoline-1(2H)-one as an off-white solid, MS (ESI) m/z: 313.9 [M+H]+.

### Intermediate A26: 7-chloro-1-(dimethylamino)isoquinoline-4-sulfonyl chloride

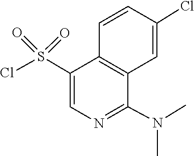 [SMILES: CN(C)c1ncc(S(=O)(=O)Cl)c2ccc(Cl)cc12]

The title compound was prepared in analogy to Intermediate A16 using dimethylamine instead of methylamine in step 1 as an orange oil, 1H NMR (300 MHz, CDCl3) δ=8.72 (s, 1H), 8.51 (d, J=9.1 Hz, 1H), 8.07 (d, J=1.6 Hz, 1H), 7.77 (dd, J=2.1, 9.2 Hz, 1H), 3.41 (s, 6H).

### Intermediate A27: 8-methylquinoline-5-sulfonyl chloride

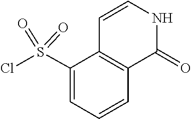 [SMILES: O=c1[nH]ccc2c(S(=O)(=O)Cl)cccc12]

Intermediate A27 is commercial (CAS 120164-05-8)

### Intermediate A28: tetralin-5-sulfonyl chloride

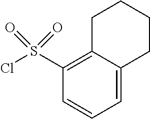 [SMILES: O=S(=O)(Cl)c1cccc2c1CCCC2]

Intermediate A28 is commercial (CAS 62686-69-5)

### Intermediates B

### Intermediate B1: 2-(4-amino-2,5-difluoro-phenyl)acetonitrile

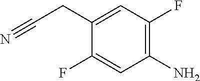 [SMILES: N#CCc1cc(F)c(N)cc1F]

Intermediate B1 is known (CAS: 2092112-51-9) and was synthesized according to WO2018/122232 page 229.

### Intermediate B2: 6-(2,2-difluoroethoxy)-5-fluoro-2-methoxy-pyridin-3-amine

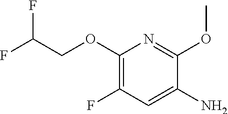

Intermediate B2 is known (CAS: 2407471-07-0) and was synthesized according to WO2019/243303 page 83.

### Intermediate B3: 6-cyclopropyl-5-fluoro-2-methoxy-pyridin-3-amine

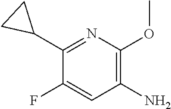

Intermediate B3 is known (CAS: 2407471-10-5) and was synthesized according to WO2019/243303 page 86.

### Intermediate B4: 2-(5-amino-3-fluoro-6-methoxy-2-pyridyl)acetonitrile

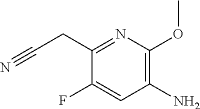

### Step 1: (6-bromo-5-fluoro-2-methoxy-3-pyridyl)amine

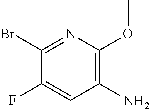

A solution of (5-fluoro-2-methoxy-3-pyridyl)amine (3.9 g, 26.07 mmol) and N-bromosuccinimide (5.16 g, 28.67 mmol) in acetonitrile (400 ml) was stirred at room temperature for 30 min. The reaction mixture was diluted with ethyl acetate, washed once with saturated NaHCO₃ solution, once with brine, dried over Na2SO4, filtered and concentrated in vacuo. The crude material was purified by flash chromatography (silica gel, 0% to 30% ethyl acetate in heptane) to afford (6-bromo-5-fluoro-2-methoxy-3-pyridyl)amine (5.26 g, 86.7%) as dark brown solid. MS (ESI) m/z: 221.0 [M+H]+.

### Step 2: (6-bromo-5-fluoro-2-methoxy-3-pyridyl)-bis(p-anisyl)amine

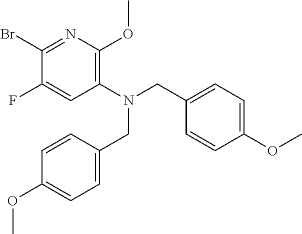 [SMILES: COc1ccc(CN(Cc2ccc(OC)cc2)c2cc(F)c(Br)nc2OC)cc1]

To a stirred solution of (6-bromo-5-fluoro-2-methoxy-3-pyridyl)amine (2.5 g, 10.75 mmol) in N,N-dimethylacetamide (25 ml) was added sodium hydride (1.29 g, 32.24 mmol) portion-wise (5×258 mg) at 0° C. After stirring at 0° C. for 30 min, 4-methoxybenzyl chloride (3.43 g, 3.0 ml, 21.49 mmol) was added. The reaction mixture was allowed to warm to room temperature and stirred for 30 min. The reaction mixture was carefully quenched with saturated NH4Cl solution, poured into water and extracted twice with ethyl acetate. The combined organic layers were washed twice with brine, dried over Na2SO4, filtered and concentrated in vacuo. The crude material was purified by flash chromatography (silica gel, 0% to 10% ethyl acetate in heptane) to afford (6-bromo-5-fluoro-2-methoxy-3-pyridyl)-bis(p-anisyl)amine (4.93 g, 99.5%) as red viscous oil.

### Step 3: [5-[bis(p-anisyl)amino]-3-fluoro-6-methoxy-2-pyridyl]methanol

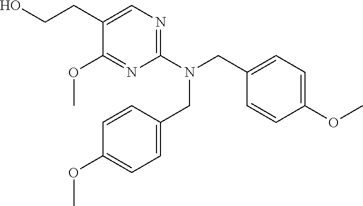 [SMILES: COc1ccc(CN(Cc2ccc(OC)cc2)c2ncc(CCO)c(OC)n2)cc1]

A solution of (6-bromo-5-fluoro-2-methoxy-3-pyridyl)-bis(p-anisyl)amine (1 g, 2.17 mmol) in toluene (20 ml) was cooled to −78° C. 1.6 M n-butyllithium (1.75 g, 2.03 mL, 3.25 mmol) was added dropwise. The resulting dark blue solution was stirred at −78° C. for 30 min. N,N-dimethylformamide (396 mg, 419 ul, 5.42 mmol) was added and stirring was continued at −78° C. for 30 min. The reaction mixture was allowed to warm to room temperature. Methanol (4 ml) was added, followed by sodium borohydride (82 mg, 2.17 mmol). The stirring was continued at room temperature for 15 min. The reaction mixture was quenched with saturated NH4Cl solution and extracted twice with ethyl acetate. The organic layers were dried over Na2SO4, filtered and concentrated in vacuo. The crude material was purified by flash chromatography (silica gel, 0% to 50% ethyl acetate in heptane) to afford [5-[bis(p-anisyl)amino]-3-fluoro-6-methoxy-2-pyridyl]methanol (297 mg, 32.5%) as light yellow viscous oil. MS (ESI) m/z: 413.3 [M+H]+.

### Step 4: [6-(chloromethyl)-5-fluoro-2-methoxy-3-pyridyl]-bis(p-anisyl)amine

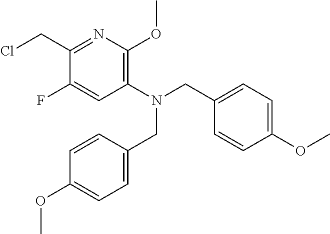 [SMILES: COc1ccc(CN(Cc2ccc(OC)cc2)c2cc(F)c(CCl)nc2OC)cc1]

To a stirred solution of [5-[bis(p-anisyl)amino]-3-fluoro-6-methoxy-2-pyridyl]methanol (144 mg, 0.342 mmol) in dichloromethane (1.5 ml) was added thionyl chloride (81.4 mg, 50 ul, 0.684 mmol) dropwise at room temperature. The reaction mixture was stirred at room temperature for 1 h. The reaction mixture was concentrated in vacuo to afford [6-(chloromethyl)-5-fluoro-2-methoxy-3-pyridyl]-bis(p-anisyl)amine (150 mg, 100%) as light brown foam, which was directly used in the next step without further purification.

### Step 5: 2-[5-[bis(p-anisyl)amino]-3-fluoro-6-methoxy-2-pyridyl]acetonitrile

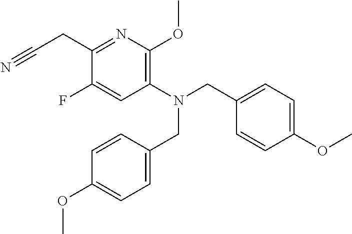 [SMILES: COc1ccc(CN(Cc2ccc(OC)cc2)c2cc(F)c(CC#N)nc2OC)cc1]

To a stirred solution of [6-(chloromethyl)-5-fluoro-2-methoxy-3-pyridyl]-bis(p-anisyl)amine (152 mg, 0.353 mmol) in dichloromethane (700 ul) was added tetrabutylammonium bromide (11.5 mg, 0.035 mmol), followed by a solution of sodium cyanide (21 mg, 0.423 mmol) in water (130 ul). After stirring the reaction mixture for 15 h at room temperature more sodium cyanide (21 mg, 0.423 mmol) was added and stirring was continued at room temperature for 15 h. The reaction mixture was diluted with dichloromethane and washed with water twice. The organic layer was dried over Na2SO4, filtered and concentrated in vacuo. The crude material was purified by flash chromatography (silica gel, 0% to 30% ethyl acetate in heptane) to afford 2-[5-[bis(p-anisyl)amino]-3-fluoro-6-methoxy-2-pyridyl]acetonitrile (87 mg, 58.5%) as colorless viscous oil. MS (ESI) m/z: 422.3 [M+H]+

### Step 6: 2-(5-amino-3-fluoro-6-methoxy-2-pyridyl)acetonitrile

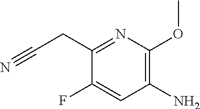

A solution of 2-[5-[bis(p-anisyl)amino]-3-fluoro-6-methoxy-2-pyridyl]acetonitrile (85 mg, 0.20 mmol) in dichloromethane (400 ul) was cooled to 0° C. Trifluoroacetic acid (1.38 g, 927 ul, 12.1 mmol) was added and the reaction mixture was stirred at 0° C. for 20 min and at room temperature for 4 h. The resulting purple solution was poured into saturated NaHCO₃ solution and extracted with ethyl acetate twice. The combined organic layers were dried over Na2SO4, filtered and concentrated in vacuo. The crude material was purified by flash chromatography (silica gel, 0% to 40% ethyl acetate in heptane) to afford 2-(5-amino-3-fluoro-6-methoxy-2-pyridyl)acetonitrile (31 mg, 85%) as a light yellow solid. MS (ESI) m/z: 182.1 [M+H]+

### Intermediate B5: 6-(difluoromethoxy)-5-fluoro-2-methoxy-pyridin-3-amine

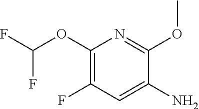

Intermediate B5 is known (CAS: 2407470-90-8) and was synthesized according to WO2019/243303 page 69.

### Intermediate B6: 5-fluoro-2-methoxy-6-(oxetan-3-yl)pyridin-3-amine

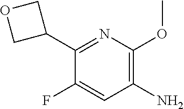

### Step 1: (6-bromo-5-fluoro-2-methoxy-3-pyridyl)amine

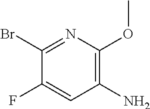

A solution of (5-fluoro-2-methoxy-3-pyridyl)amine (3.9 g, 26.07 mmol) and N-bromosuccinimide (5.16 g, 28.67 mmol) in acetonitrile (400 ml) was stirred at room temperature for 30 min. The reaction mixture was diluted with ethyl acetate, washed once with saturated NaHCO₃ solution, once with brine, dried over Na2SO4, filtered and concentrated in vacuo. The crude material was purified by flash chromatography (silica gel, 0% to 30% ethyl acetate in heptane) to afford (6-bromo-5-fluoro-2-methoxy-3-pyridyl)amine (5.26 g, 86.7%) as dark brown solid. MS (ESI) m/z: 221.0 [M+H]+.

### Step 2: 5-fluoro-2-methoxy-6-(oxetan-3-yl)pyridin-3-amine

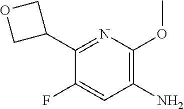

A solution of [4,4′-bis(1,1-dimethylethyl)-2,2′-bipyridine-N1,N1′]bis[3,5-difluoro-2-[5-(trifluoromethyl)-2-pyridinyl-N]phenyl-C]iridium(III) hexafluorophosphate (Ir[dF(CF3)ppy]2(dtbpy))PF6, CAS 870987-63-6, 7.6 mg, 0.007 mmol, 0.010 eq, 6.9 M in dichloromethane) was added to a vial and the solvent was evaporated. Sodium carbonate (142 mg, 1.36 mmol, 2 eq), (6-bromo-5-fluoro-2-methoxy-3-pyridyl)amine (150 mg, 0.679 mmol, 1 eq), 3-bromooxetane (186 mg, 113 ul, 1.36 mmol, 2 eq) and tris(trimethylsilyl)silane (226 mg, 281 ul, 0.909 mmol, 1.34 eq) were added. Ethylene glycol dimethyl ether (extra dry, 11 ml) was added and the reaction mixture was degassed by bubbling argon through the mixture for 2 min. Finally, nickel(ii) chloride ethylene glycol dimethyl ether complex (NiCl2*glyme/dtbbpy, CAS 29046-78-4, 746 ug, 0.003 mmol, 0.005 eq) and 4,4′-bis(tert-butyl)-2,2′-bipyridine (911 ug, 0.003 mmol, 0.005 eq) was added and the reaction was degassed again for 5 min. The vial was placed in a photoreactor and irradiated with 450 nm for 14 h (stirred at 900 rpm, LED 100% power). The reaction mixture was filtered and concentrated in vacuo. The residue was dissolved in dichloromethane and purified by column chromatography (silica gel, 0 to 40% ethyl acetate in heptane) to afford 5-fluoro-2-methoxy-6-(oxetan-3-yl)pyridin-3-amine (47 mg, 35%) as an off-white solid. MS (ESI) m/z: 199.1 [M+H]+.

### Intermediate B7: 5-(2,2-difluoroethyl)-4,6-dimethoxy-pyrimidin-2-amine

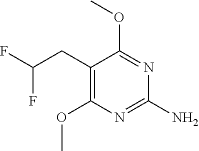

### Step 1: diethyl 2-(2,2-difluoroethyl)propanedioate

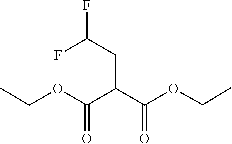

Diethyl malonate (75.8 ml, 500 mmol) was combined with tetrahydrofuran (450 ml). Sodium ethoxide (prepared from ethanol (150 mL) and sodium (11.48 g, 500 mmol)) was added at room temperature and the reaction mixture was stirred 15 min at room temperature. A solution of 2,2-difluoroethyl trifluoromethanesulfonate (76 ml, 500 mmol) in tetrahydrofuran (10 ml) was added slowly. The reaction mixture was stirred for 18 hours at 20° C., then cooled to 0° C., quenched with a saturated ammonium chloride solution and extracted twice with ethyl acetate. The organic layers were washed with brine, dried over sodium sulfate, filtered and concentrated in vacuo to provide the title compound (100.5 g, 90% yield). MS (ESI) m/z=225.0 [M+H]+

### Step 2: 2-amino-5-(2,2-difluoroethyl)pyrimidine-4,6-diol

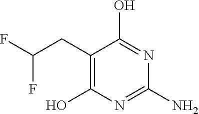 [SMILES: Nc1nc(O)c(CC(F)F)c(O)n1]

To a stirred solution of diethyl 2-(2,2-difluoroethyl)propanedioate (46.8 g, 209 mmol) in ethanol (5 mL) was added guanidine hydrochloride (19.9 g, 208 mmol), followed by sodium ethoxide (prepared from ethanol and sodium (14.38 g, 625 mmol)). The resulting orange suspension was heated to 80° C. and stirred for 4 hours. The reaction mixture was concentrated by half, 50 ml of water was added, followed by acetic acid (42.57 g, 709 mmol). The mixture was heated to 80° C. and stirred for 10 min, then cooled to room temperature. The solid product was filtered off, washed successively with water, ethanol and methyl tert-butyl ether to provide the title compound (22.3 g, 50% yield). MS (ESI) m/z=192.0 [M+H]+

### Step 3: 4,6-dichloro-5-(2,2-difluoroethyl)pyrimidin-2-amine

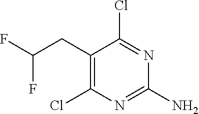 [SMILES: Nc1nc(Cl)c(CC(F)F)c(Cl)n1]

2-amino-5-(2,2-difluoroethyl)pyrimidine-4,6-diol (13.2 g, 69.1 mmol) was suspended in phosphorus oxychloride (80.5 ml, 863 mmol). The reaction mixture was heated to 100° C. and stirred for 18 hours and concentrated in vacuo. The residue was diluted with ethyl acetate and carefully poured into ice/saturated sodium bicarbonate solution. The resulting biphasic mixture was stirred at room temperature for 5 min and extracted twice with ethyl acetate. The organic layers were dried over sodium sulfate, filtered and concentrated in vacuo. The residue was purified by flash chromatography over silica gel to provide the title compound (7.35 g, 47% yield). MS (ESI) m/z=227.8 [M+H]+

### Step 4: 5-(2,2-difluoroethyl)-4,6-dimethoxy-pyrimidin-2-amine

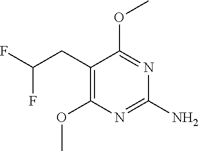

In a sealed tube, a mixture of 4,6-dichloro-5-(2,2-difluoroethyl)pyrimidin-2-amine (7.6 g, 33.33 mmol) and sodium methylate (prepared from sodium (7.66 g, 333.29 mmol) in methanol (50 ml)) was heated to 75° C. and stirred for 18 hours. The reaction mixture was quenched with water and extracted twice with ethyl acetate. The organic layers were dried over sodium sulfate, filtered and concentrated in vacuo to provide the title compound as a light yellow solid (6.6 g, 86% yield). MS (ESI) m/z=220.0 [M+H]+

### Intermediate B8: 5-(difluoromethoxy)-4,6-dimethoxy-pyrimidin-2-amine

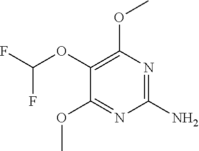

### Step 1: 5-bromo-4,6-dimethoxy-pyrimidin-2-amine

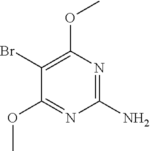

To a stirred solution of (4,6-dimethoxypyrimidin-2-yl)amine (7 g, 44.22 mmol, CAS: 36315-01-2) in acetonitrile (100 ml) was added a solution of N-bromosuccinimide (10.33 g, 57.48 mmol) in acetonitrile (100 ml) dropwise at room temperature. The reaction mixture was stirred at room temperature for 30 min. The resulting white suspension was diluted with ethyl acetate and washed with water. The organic layer was dried over sodium sulfate, filtered, diluted with heptane and concentrated in vacuo. The precipitate was filtered off and washed with heptane to provide the title compound as a white solid (9.26 g, 87% yield). MS (ESI) m/z=234.1 [M+H]+

### Step 2: 5-bromo-4,6-dimethoxy-N,N-bis[(4-methoxyphenyl)methyl]pyrimidin-2-amine

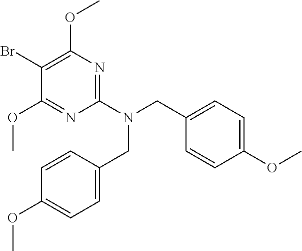 [SMILES: COc1ccc(CN(Cc2ccc(OC)cc2)c2nc(OC)c(Br)c(OC)n2)cc1]

A solution of 5-bromo-4,6-dimethoxy-pyrimidin-2-amine (517 mg, 2.21 mmol) in N,N-dimethylacetamide (9 ml) was cooled to 0° C. Sodium hydride (265 mg, 6.63 mmol) was added portionwise (3×88 mg). The stirring was continued at 0° C. for 30 min. 4-methoxybenzyl chloride (706 mg, 609 ul, 4.42 mmol) was added dropwise. The reaction mixture was allowed to warm to room temperature, stirred for 1 hour, carefully quenched with a saturated ammonium chloride solution, poured into water and extracted twice with ethyl acetate. The organic layers were dried over sodium sulfate, filtered and concentrated in vacuo. The residue was purified by flash chromatography over silica gel using a gradient ethyl acetate/heptane 0-20% to provide the title compound as a white solid (1.11 g, 100% yield). MS (ESI) m/z=476.2 [M+H]+

### Step 3: 2-[bis[(4-methoxyphenyl)methyl]amino]-4,6-dimethoxy-pyrimidin-5-ol

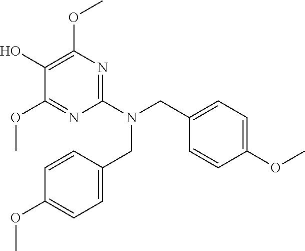 [SMILES: COc1ccc(CN(Cc2ccc(OC)cc2)c2nc(OC)c(O)c(OC)n2)cc1]

To a colorless solution of 5-bromo-4,6-dimethoxy-N,N-bis[(4-methoxyphenyl)methyl]-pyrimidin-2-amine (500 mg, 1 mmol) in tetrahydrofuran (3.5 ml) was added a 1.6 M n-butyllithium solution in hexanes (700 mg, 814 ul, 1.3 mmol) dropwise at −78° C. The resulting yellow solution was stirred at −78° C. for 30 min. Trimethyl borate (156 mg, 167 ul, 1.5 mmol) was added dropwise and the stirring was continued at −78° C. for 1.5 hours. The reaction mixture was allowed to warm to 0° C. and acetic acid (120 mg, 115 ul, 2 mmol) was added dropwise, followed by hydrogen peroxide 35% (146 mg, 132 ul, 1.5 mmol). The stirring was continued at 0° C. for 1.5 hours. The resulting pink suspension was poured into a 0.1 N sodium thiosulfate solution and extracted twice with ethyl acetate. The organic layers were washed with brine, dried over sodium sulfate, filtered and concentrated in vacuo. The residue was purified by flash chromatography over silica gel using a gradient ethyl acetate/heptane 0-30% to provide the title compound as a light yellow viscous oil (194 mg, 47% yield). MS (ESI) m/z=412.3 [M+H]+

### Step 4: 5-(difluoromethoxy)-4,6-dimethoxy-N,N-bis[(4-methoxyphenyl)methyl]pyrimidin-2-amine

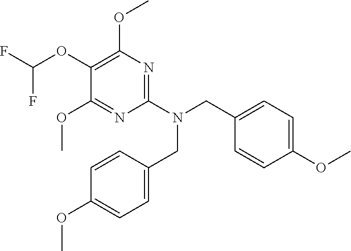 [SMILES: COc1ccc(CN(Cc2ccc(OC)cc2)c2nc(OC)c(OC(F)F)c(OC)n2)cc1]

To a solution of 2-[bis[(4-methoxyphenyl)methyl]amino]-4,6-dimethoxy-pyrimidin-5-ol (130 mg, 0.316 mmol) in acetonitrile (5 ml) was added a 5 M potassium hydroxide solution (1.26 ml, 6.32 mmol) dropwise at 0° C., followed by addition of bromodifluoromethyl diethylphosphonate (169 mg, 112 ul, 0.632 mmol) in acetonitrile (1 ml) dropwise at 0° C. The reaction mixture was stirred at 0° C. for 10 min. The resulting light yellow biphasic mixture was poured into water and extracted twice with ethyl acetate. The organic layers were dried over sodium sulfate, filtered and concentrated in vacuo. The residue was purified by flash chromatography over silica gel using a gradient ethyl acetate/heptane 0-30% to provide the title compound as a white solid (57 mg, 39% yield). MS (ESI) m/z=462.3 [M+H]+

### Step 5: 5-(difluoromethoxy)-4,6-dimethoxy-pyrimidin-2-amine

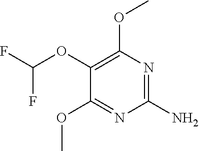

To a stirred solution of 5-(difluoromethoxy)-4,6-dimethoxy-N,N-bis[(4-methoxyphenyl)methyl]pyrimidin-2-amine (56 mg, 0.121 mmol) in dichloromethane (100 ul) was added trifluoroacetic acid (839 mg, 563 ul, 7.28 mmol). The reaction mixture was stirred at room temperature for 40 hours, at 50° C. for 6 hours and concentrated in vacuo. The residue was poured into sat. NaHCO₃ and extracted twice with ethyl acetate. The organic layers were dried over sodium sulfate, filtered and concentrated in vacuo. The residue was purified by flash chromatography over silica gel using a gradient ethyl acetate/heptane 0-100% to provide the title compound as a white solid (24 mg, 89% yield). MS (ESI) m/z=222.1 [M+H]+

### Intermediate B9: 5-(2,2-difluoroethoxy)-3-fluoro-6-methoxy-pyridin-2-amine

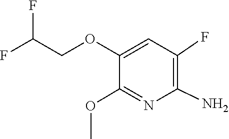

Intermediate B9 is known (CAS 2404661-29-4) and was synthesized according to WO2019243398 page 55.

### Intermediate B10: 5-(2-fluoroethoxy)-4-methoxy-pyrimidin-2-amine

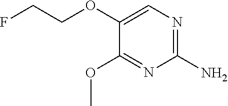

### Step 1: (5-bromo-4-methoxy-pyrimidin-2-yl)-bis(p-anisyl)amine

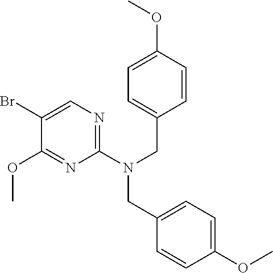 [SMILES: COc1ccc(CN(Cc2ccc(OC)cc2)c2ncc(Br)c(OC)n2)cc1]

A suspension of 5-bromo-2-chloro-4-methoxy-pyrimidine (1.02 g, 4.48 mmol, CAS: 57054-92-9), bis(p-anisyl)amine (1.29 g, 4.92 mmol) and N-ethyl diisopropylamine (858 ul, 4.92 mmol) in acetonitrile (20 ml) was heated at 70° C. for 2 days. The resulting solution was poured into a saturated aqueous sodium bicarbonate solution and extracted twice with ethyl acetate. The organic layers were dried over sodium sulfate, filtered and dried in vacuo. The residue was purified by flash chromatography over silica gel using a gradient ethyl acetate/heptane 0-20% to provide the title compound as a colorless viscous oil (998 mg, 50% yield). MS (ESI): m/z=446.2 [M+H]+

### Step 2: [4-methoxy-5-(4,4,5,5-tetramethyl-1,3,2-dioxaborolan-2-yl)pyrimidin-2-yl]-bis(p-anisyl)amine

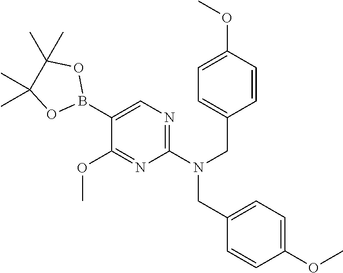

A suspension of (5-bromo-4-methoxy-pyrimidin-2-yl)-bis(p-anisyl)amine (500 mg, 1.13 mmol), bis(pinacolato)diboron (354 mg, 1.35 mmol) and potassium acetate (335 mg, 3.38 mmol) in 1,4-dioxane (10 ml) was purged with argon for 5 min. dichloro[1,1′-bis(diphenylphosphino)ferrocene]palladium(II) dichloromethane adduct (91.9 mg, 0.113 mmol) was added. The reaction mixture was heated to 90° C. and stirred for 16 hours. The resulting dark suspension was poured into ethyl acetate and washed once with saturated sodium chloride solution. The organic layer was dried over sodium sulfate, filtered and concentrated in vacuo. The residue was purified by column chromatography on silica gel using a gradient ethyl acetate/heptane 0-30% to provide the title compound as a colorless viscous oil (157 mg, 29% yield). MS (ESI): m/z=492.4 [M+H]+

### Step 3: 2-[bis(p-anisyl)amino]-4-methoxy-pyrimidin-5-ol

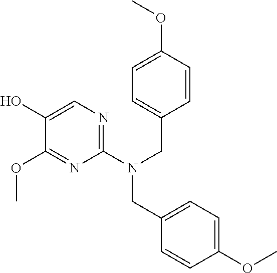 [SMILES: COc1ccc(CN(Cc2ccc(OC)cc2)c2ncc(O)c(OC)n2)cc1]

A solution of [4-methoxy-5-(4,4,5,5-tetramethyl-1,3,2-dioxaborolan-2-yl)pyrimidin-2-yl]-bis(p-anisyl)amine (130 mg, 0.265 mmol) in tetrahydrofuran (2.5 mL) was cooled to 0° C. Hydrogen peroxide 35% (500 ul, 5.71 mmol) was added. The reaction mixture was stirred at 0° C. for 15 min, allowed to warm to rt and stirred for 3 hours. The reaction mixture was poured into cold 0.1 N sodium sulfite solution and extracted twice with ethyl acetate. The organic layers were washed twice with brine, dried over sodium sulfate, filtered and concentrated in vacuo to provide the title compound as a light yellow viscous oil (103 mg, 100% yield). MS (ESI): m/z=382.3 [M+H]+

### Step 4: [5-(2-fluoroethoxy)-4-methoxy-pyrimidin-2-yl]-bis(p-anisyl)amine

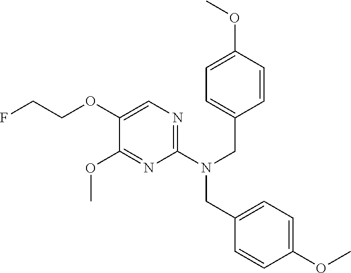 [SMILES: COc1ccc(CN(Cc2ccc(OC)cc2)c2ncc(OCCF)c(OC)n2)cc1]

A suspension of 2-[bis(p-anisyl)amino]-4-methoxy-pyrimidin-5-ol (100 mg, 0.236 mmol), potassium carbonate (98.82 mg, 0.708 mmol) and 1-bromo-2-fluoroethane (61.14 mg, 35.75 uL, 0.472 mmol) in acetonitrile (2.5 ml) was stirred at room temperature for 15 min and at 80° C. for 6 hours. The reaction mixture was poured into water and extracted twice with ethyl acetate. The organic layers were dried over sodium sulfate, filtered and concentrated in vacuo. The residue was purified by flash chromatography on silica gel using a gradient ethyl acetate/heptane 0-30% to provide the title compound as a colorless viscous oil (22 mg, 22% yield). MS (ESI): m/z=428.3 [M+H]+

### Step 5: [5-(2-fluoroethoxy)-4-methoxy-pyrimidin-2-yl]amine

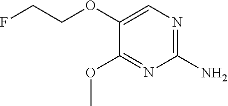

A solution of [5-(2-fluoroethoxy)-4-methoxy-pyrimidin-2-yl]-bis(p-anisyl)amine (87 mg, 0.204 mmol) in dichloromethane (500 ul) was cooled to 0° C. Trifluoroacetic acid (1.41 g, 945 ul, 12.2 mmol) was added. The reaction mixture was allowed to warm to room temperature and stirred for 16 hours and at 55° C. for two additional hours. The resulting purple solution was poured into a saturated aqueous sodium bicarbonate solution and extracted twice with ethyl acetate. The organic layers were dried over sodium sulfate, filtered and concentrated in vacuo. The residue was purified by flash chromatography on silica gel using a gradient ethyl acetate/heptane 0-100% to provide the title compound as an off-white solid (27 mg, 71% yield). MS (ESI): m/z=188.1 [M+H]+

### Intermediate B11: 2,6-bis(difluoromethoxy)-5-fluoro-pyridin-3-amine

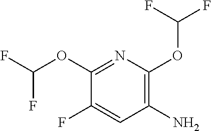 [SMILES: Nc1cc(F)c(OC(F)F)nc1OC(F)F]

### Step 1: 6-(difluoromethoxy)-5-fluoro-3-nitro-pyridin-2-ol

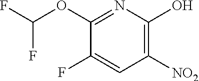 [SMILES: O=[N+]([O-])c1cc(F)c(OC(F)F)nc1O]

To a solution of 2-(difluoromethoxy)-3-fluoro-6-methoxy-5-nitro-pyridine (CAS 2407470-89-5, see WO2020254289, 1.7 g, 7.14 mmol) in dichloromethane (24 ml) was added boron tribromide (3.34 ml, 35.7 mmol) at 0° C., then the reaction mixture was stirred at 20° C. for 1 h. The reaction mixture was poured into water (150 ml) and extracted with ethyl acetate (50 ml×3). The organic layers were washed with brine, dried over anhydrous sodium sulfate and concentrated under reduced pressure. The residue was purified by column chromatography (petroleum ether to petroleum ether/ethyl acetate=2:1) to give 6-(difluoromethoxy)-5-fluoro-3-nitro-pyridin-2-ol (1.48 g, 93% yield) as yellow oil. 1H NMR (400 MHz, CDCl3) δ ppm 7.31-7.69 (m, 1H) 8.33 (d, J=7.70 Hz, 1H) 11.35 (br s, 1H)

### Step 2: 2,6-bis(difluoromethoxy)-3-fluoro-5-nitro-pyridine

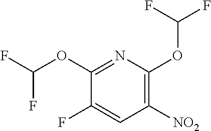 [SMILES: O=[N+]([O-])c1cc(F)c(OC(F)F)nc1OC(F)F]

To the solution of 6-(difluoromethoxy)-5-fluoro-3-nitro-pyridin-2-ol (1.48 g, 6.6 mmol) in acetonitrile (20 ml) was added a solution of potassium hydroxide (3.71 g, 66.0 mmol) in water (5 ml), and diethyl (bromodifluoromethyl)phosphonate (10.580 g, 39.63 mmol) at 40° C., then the reaction mixture was stirred at 40° C. for 16 h. The reaction mixture was extracted with dichloromethane (60 ml×3). The organic layers was washed with brine, dried over anhydrous sodium sulfate and concentrated under reduced pressure. The residue was purified by column chromatography (silica gel, petroleum ether to petroleum ether/ethyl acetate=5:1) to give 2,6-bis(difluoromethoxy)-3-fluoro-5-nitro-pyridine (2.38 g, 46% yield) as light yellow oil. 1H NMR (400 MHz, DMSO-d6) δ ppm 7.64-8.12 (m, 2H) 8.98 (d, J=8.93 Hz, 1H).

### Step 3: 2,6-bis(difluoromethoxy)-5-fluoro-pyridin-3-amine

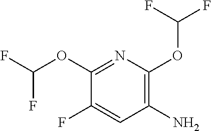 [SMILES: Nc1cc(F)c(OC(F)F)nc1OC(F)F]

To the mixture of 2,6-bis(difluoromethoxy)-3-fluoro-5-nitro-pyridine (380 mg, 1.39 mmol) in ethanol (12 ml) and water (3 ml) was added iron (389 mg, 6.93 mmol) and ammonium chloride (367 mg, 6.93 mmol) at 25° C., then the reaction mixture was stirred at 25° C. for 1 h. The reaction mixture was diluted with water (3 ml) and extracted with ethyl acetate (3×10 ml). The combined organic layers were washed with saturated NaCl solution (5 ml), dried over Na2SO4, filtered and concentrated under reduced pressure to give a residue which was purified by preparative TLC (SiO2, petroleum ether/ethyl acetate=2:1) to give 2,6-bis(difluoromethoxy)-5-fluoro-pyridin-3-amine (41 mg, 12% yield) as a yellow oil. MS (ESI): m/z=244.8 [M+H]+.

### EXAMPLES

### Example 1: N-(4-(cyanomethyl)-2,5-difluorophenyl)naphthalene-1-sulfonamide

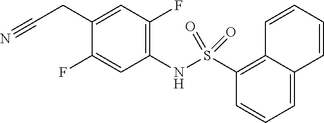 [SMILES: N#CCc1cc(F)c(NS(=O)(=O)c2cccc3ccccc23)cc1F]

To naphthalene-1-sulfonyl chloride (50 mg, 0.223 mmol, intermediate A1) was added 2-(4-amino-2,5-difluoro-phenyl)acetonitrile (25 mg, 0.149 mmol, intermediate B1) followed by pyridine, extra dry (1 ml). The reaction mixture was stirred room temperature for 2 hours, then the solvent was evaporated. The crude material was purified by preparative HPLC (column: YMC-Triart C18, 12 nm, 5 μm, 100×30 mm, gradient acetonitrile/water+0.1% HCOOH) to afford the title compound (37 mg, 69%) as white powder, MS (ESI): m/z=357.2 [M−H]⁻.

The following Examples 2-39 were prepared in analogy to Example 1 by coupling the indicated sulfonylchloride intermediates A and amine intermediates B.

| | | | | | MS |
|---|---|---|---|---|---|
| | | | | | (ESI): |
| Ex. | Structure | Name | Int. A | Int. B | m/z |
| 2 | 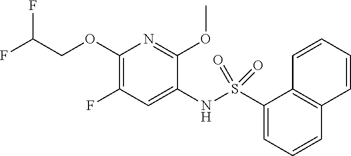 [SMILES: COc1nc(OCC(F)F)c(F)cc1NS(=O)(=O)c1cccc2ccccc12] | N-[6-(2,2-difluoroethoxy)- 5-fluoro-2-methoxy-3- pyridyl]naphthalene-1- sulfonamide | A1 | B2 | 413.2 [M + H]⁺ |
| 3 | 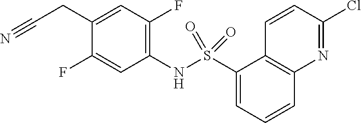 [SMILES: N#CCc1cc(F)c(NS(=O)(=O)c2cccc3nc(Cl)ccc23)cc1F] | 2-chloro-N-[ 4- (cyanomethyl)-2,5-difluoro- phenyl]quinoline-5- sulfonamide | A3 | B1 | 394.3 [M + H]⁺ |
| 4 | 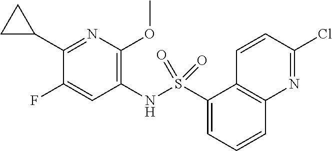 [SMILES: COc1nc(C2CC2)c(F)cc1NS(=O)(=O)c1cccc2nc(Cl)ccc12] | 2-chloro-N-(6-cyclopropyl- 5-fluoro-2-methoxy-3- pyridyl)quinoline-5- sulfonamide | A3 | B3 | 408.1 [M + H]⁺ |
| 5 | 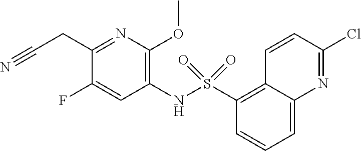 [SMILES: COc1nc(CC#N)c(F)cc1NS(=O)(=O)c1cccc2nc(Cl)ccc12] | 2-chloro-N-[6- (cyanomethyl)-5-fluoro-2- methoxy-3- pyridyl]quinoline-5- sulfonamide | A3 | B4 | 407.0 [M + H]⁺ |
| 6 | 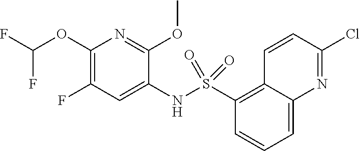 [SMILES: COc1nc(OC(F)F)c(F)cc1NS(=O)(=O)c1cccc2nc(Cl)ccc12] | 2-chloro-N-[6- (difluoromethoxy)-5-fluoro- 2-methoxy-3- pyridyl]quinoline-5- sulfonamide | A3 | B5 | 434.1 [M + H]⁺ |
| 7 | 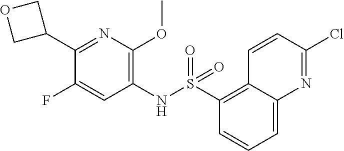 [SMILES: COc1nc(C2COC2)c(F)cc1NS(=O)(=O)c1cccc2nc(Cl)ccc12] | 2-chloro-N-[5-fluoro-2- methoxy-6-(oxetan-3-yl)-3- pyridyl]quinoline-5- sulfonamide | A3 | B6 | 424.1 [M + H]⁺ |
| 8 | 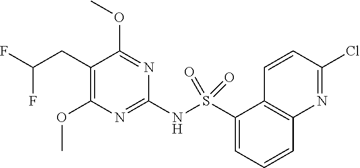 [SMILES: COc1nc(NS(=O)(=O)c2cccc3nc(Cl)ccc23)nc(OC)c1CC(F)F] | 2-chloro-N-[5-(2,2- difluoroethyl)-4,6- dimethoxy-pyrimidin-2- yl]quinoline-5-sulfonamide | A3 | B7 | 445.2 [M + H]⁺ |
| 9 | 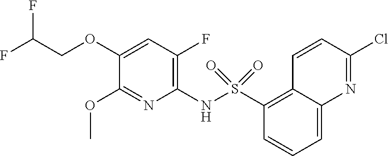 [SMILES: COc1nc(NS(=O)(=O)c2cccc3nc(Cl)ccc23)c(F)cc1OCC(F)F] | 2-chloro-N-[5-(2,2- difluoroethoxy)-3-fluoro-6- methoxy-2- pyridyl]quinoline-5- sulfonamide | A3 | B9 | 448.1 [M + H]⁺ |
| 10 | 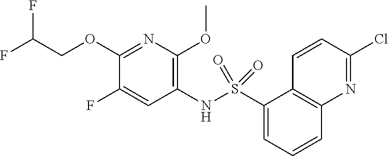 [SMILES: COc1nc(OCC(F)F)c(F)cc1NS(=O)(=O)c1cccc2nc(Cl)ccc12] | 2-chloro-N-[6-(2,2- difluoroethoxy)-5-fluoro-2- methoxy-3- pyridyl]quinoline-5- sulfonamide | A3 | B2 | 448.3 [M + H]⁺ |
| 11 | 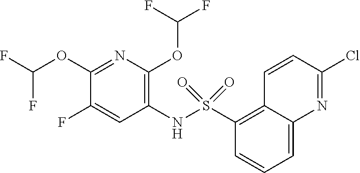 [SMILES: O=S(=O)(Nc1cc(F)c(OC(F)F)nc1OC(F)F)c1cccc2nc(Cl)ccc12] | N-[2,6- bis(difluoromethoxy)-5- fluoro-3-pyridyl]-2-chloro- quinoline-5-sulfonamide | A3 | B11 | 470.1 [M + H]⁺ |
| 12 | 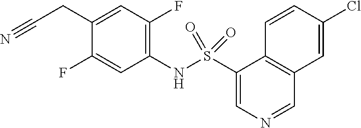 [SMILES: N#CCc1cc(F)c(NS(=O)(=O)c2cncc3cc(Cl)ccc23)cc1F] | 7-chloro-N-[4- (cyanomethyl)-2,5-difluoro- phenyllisoquinoline-4- sulfonamide | A4 | B1 | 394.1 [M + H]⁺ |
| 13 | 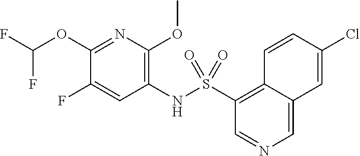 [SMILES: COc1nc(OC(F)F)c(F)cc1NS(=O)(=O)c1cncc2cc(Cl)ccc12] | 7-chloro-N-[6- (difluoromethoxy)-5-fluoro- 2-methoxy-3- pyridyl]isoquinoline-4- sulfonamide | A4 | B5 | 434.0 [M + H]⁺ |
| 14 | 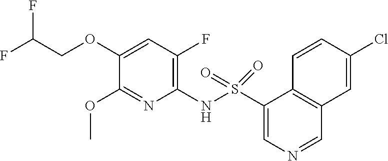 [SMILES: COc1nc(NS(=O)(=O)c2cncc3cc(Cl)ccc23)c(F)cc1OCC(F)F] | 7-chloro-N-[5-(2,2- difluoroethoxy)-3-fluoro-6- methoxy-2- pyridyl]isoquinoline-4- sulfonamide | A4 | B9 | 447.8 [M + H]⁺ |
| 15 | 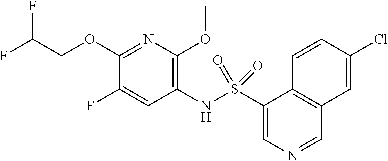 [SMILES: COc1nc(OCC(F)F)c(F)cc1NS(=O)(=O)c1cncc2cc(Cl)ccc12] | 7-chloro-N-[6-(2,2- difluoroethoxy)-5-fluoro-2- methoxy-3- pyridyl]isoquinoline-4- sulfonamide | A4 | B2 | 448.2 [M + H]⁺ |
| 16 | 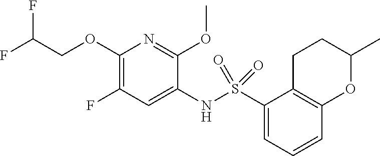 [SMILES: COc1nc(OCC(F)F)c(F)cc1NS(=O)(=O)c1cccc2c1CCC(C)O2] | N-[6-(2,2-difluoroethoxy)- 5-fluoro-2-methoxy-3- pyridyl]-2-methyl- chromane-5-sulfonamide | A6 | B2 | 433.0 [M + H]⁺ |
| 17 | 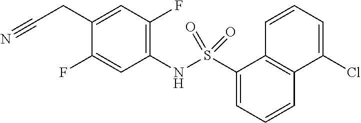 [SMILES: N#CCc1cc(F)c(NS(=O)(=O)c2cccc3c(Cl)cccc23)cc1F] | 5-chloro-N-[4- (cyanomethyl)-2,5-difluoro- phenyl]naphthalene-1- sulfonamide | A10 | B1 | 391.0 [M + H]⁺ |
| 18 | 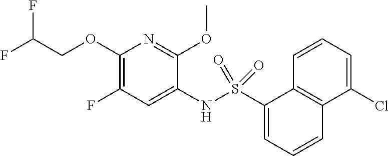 [SMILES: COc1nc(OCC(F)F)c(F)cc1NS(=O)(=O)c1cccc2c(Cl)cccc12] | 5-chloro-N-[6-(2,2- difluoroethoxy)-5-fluoro-2- methoxy-3- pyridyl]naphthalene-1- sulfonamide | A10 | B2 | 445.0 [M − H]⁻ |
| 19 | 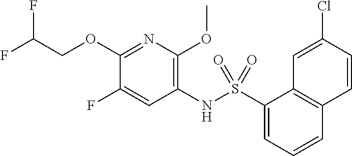 [SMILES: COc1nc(OCC(F)F)c(F)cc1NS(=O)(=O)c1cccc2ccc(Cl)cc12] | 7-chloro-N-[6-(2,2- difluoroethoxy)-5-fluoro-2- methoxy-3- pyridyl]naphthalene-1- sulfonamide | A11 | B2 | 447.2 [M + H]⁺ |
| 20 | 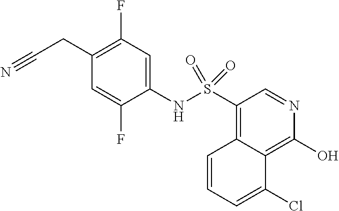 [SMILES: N#CCc1cc(F)c(NS(=O)(=O)c2cnc(O)c3c(Cl)cccc23)cc1F] | 8-chloro-N-[4- (cyanomethyl)-2,5-difluoro- phenyl]-1-hydroxy- isoquinoline-4-sulfonamide | A12 | B1 | 410.2 [M + H]⁺ |
| 21 | 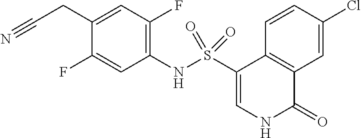 [SMILES: N#CCc1cc(F)c(NS(=O)(=O)c2c[nH]c(=O)c3cc(Cl)ccc23)cc1F] | 7-chloro-N-[4- (cyanomethyl)-2,5-difluoro- phenyl]-1-keto-2H- isoquinoline-4-sulfonamide | A13 | B1 | 408.2 [M + H]⁺ |
| 22 | 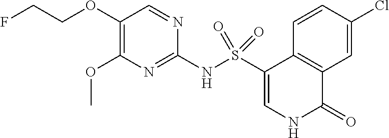 [SMILES: COc1nc(NS(=O)(=O)c2c[nH]c(=O)c3cc(Cl)ccc23)ncc1OCCF] | 7-chloro-N-[5-(2- fluoroethoxy)-4-methoxy- pyrimidin-2-yl]-1-keto-2H- isoquinoline-4-sulfonamide | A13 | B10 | 429.2 [M + H]⁺ |
| 23 | 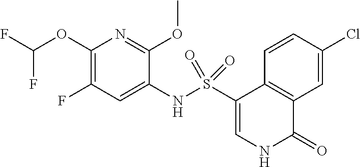 [SMILES: COc1nc(OC(F)F)c(F)cc1NS(=O)(=O)c1c[nH]c(=O)c2cc(Cl)ccc12] | 7-chloro-N-[6- (difluoromethoxy)-5-fluoro- 2-methoxy-3-pyridyl]-1- keto-2H-isoquinoline-4- sulfonamide | A13 | B5 | 450.2 [M + H]⁺ |
| 24 | 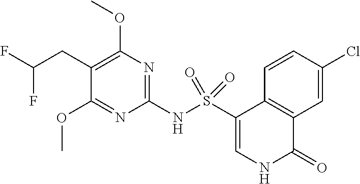 [SMILES: COc1nc(NS(=O)(=O)c2c[nH]c(=O)c3cc(Cl)ccc23)nc(OC)c1CC(F)F] | 7-chloro-N-[5-(2,2- difluoroethyl)-4,6- dimethoxy-pyrimidin-2-yl]- 1-keto-2H-isoquinoline-4- sulfonamide | A13 | B7 | 459.2 [M − H]⁻ |
| 25 | 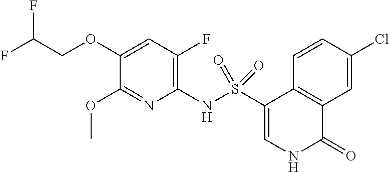 [SMILES: COc1nc(NS(=O)(=O)c2c[nH]c(=O)c3cc(Cl)ccc23)c(F)cc1OCC(F)F] | 7-chloro-N-[5-(2,2- difluoroethoxy)-3-fluoro-6- methoxy-2-pyridyl]-1-oxo- 2H-isoquinoline-4- sulfonamide | A13 | B9 | 463.9 [M + H]⁺ |
| 26 | 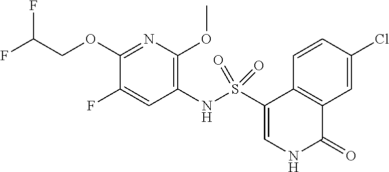 [SMILES: COc1nc(OCC(F)F)c(F)cc1NS(=O)(=O)c1c[nH]c(=O)c2cc(Cl)ccc12] | 7-chloro-N-[4-(2,2- difluoroethoxy)-5-fluoro-2- methoxy-phenyl]-1-keto- 2H-isoquinoline-4- sulfonamide | A13 | B2 | 464.2 [M + H]⁺ |
| 27 | 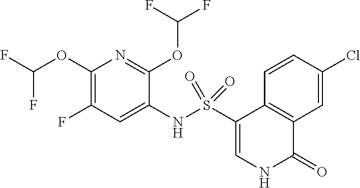 [SMILES: O=c1[nH]cc(S(=O)(=O)Nc2cc(F)c(OC(F)F)nc2OC(F)F)c2ccc(Cl)cc12] | N-[2,6- bis(difluoromethoxy)-5- fluoro-3-pyridyl]-7-chloro- 1-keto-2H-isoquinoline-4- sulfonamide | A13 | B11 | 486.1 [M + H]⁺ |
| 28 | 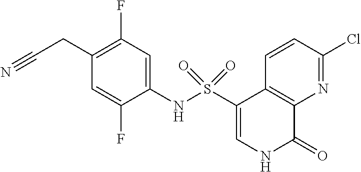 [SMILES: N#CCc1cc(F)c(NS(=O)(=O)c2c[nH]c(=O)c3nc(Cl)ccc23)cc1F] | 2-chloro-N-[4- (cyanomethyl)-2,5-difluoro- phenyl]-8-oxo-7H-1,7- naphthyridine-5- sulfonamide | A14 | B1 | 411.0 [M + H]⁺ |
| 29 | 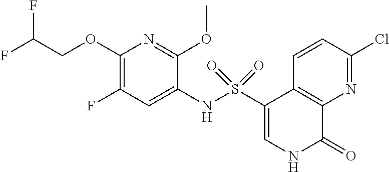 [SMILES: COc1nc(OCC(F)F)c(F)cc1NS(=O)(=O)c1c[nH]c(=O)c2nc(Cl)ccc12] | 2-chloro-N-[6-(2,2- difluoroethoxy)-5-fluoro-2- methoxy-3-pyridyl]-8-keto- 7H-1,7-naphthyridine-5- sulfonamide | A14 | B2 | 465.1 [M + H]⁺ |
| 30 | 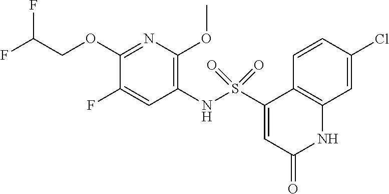 [SMILES: COc1nc(OCC(F)F)c(F)cc1NS(=O)(=O)c1cc(=O)[nH]c2cc(Cl)ccc12] | 7-chloro-N-[6-(2,2- difluoroethoxy)-5-fluoro-2- methoxy-3-pyridyl]-2-keto- 1H-quinoline-4-sulfonamide | A15 | B2 | 464.2 [M + H]⁺ |
| 31 | 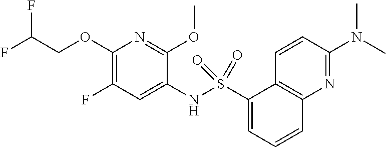 [SMILES: COc1nc(OCC(F)F)c(F)cc1NS(=O)(=O)c1cccc2nc(N(C)C)ccc12] | N-[6-(2,2-difluoroethoxy)- 5-fluoro-2-methoxy-3- pyridyl]-2- (dimethylamino)quinoline- 5-sulfonamide | A18 | B2 | 457.3 [M + H]⁺ |
| 32 | 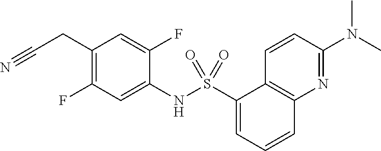 [SMILES: CN(C)c1ccc2c(S(=O)(=O)Nc3cc(F)c(CC#N)cc3F)cccc2n1] | N-[4-(cyanomethyl)-2,5- difluoro-phenyl]-2- (dimethylamino)quinoline- 5-sulfonamide | A18 | B1 | 403.2 [M + H]⁺ |
| 33 | 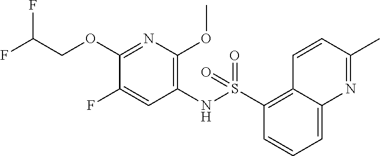 [SMILES: COc1nc(OCC(F)F)c(F)cc1NS(=O)(=O)c1cccc2nc(C)ccc12] | N-[6-(2,2-difluoroethoxy)- 5-fluoro-2-methoxy-3- pyridyl]-2-methyl- quinoline-5-sulfonamide | A8 | B2 | 428.2 [M + H]⁺ |
| 34 | 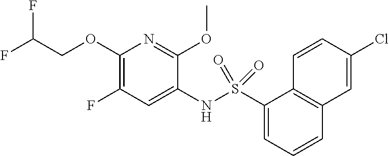 [SMILES: COc1nc(OCC(F)F)c(F)cc1NS(=O)(=O)c1cccc2cc(Cl)ccc12] | 6-chloro-N-[6-(2,2- difluoroethoxy)-5-fluoro-2- methoxy-3- pyridyl]naphthalene-1- sulfonamide | A9 | B2 | 447.1 [M + H]⁺ |
| 35 | 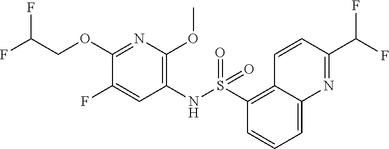 [SMILES: COc1nc(OCC(F)F)c(F)cc1NS(=O)(=O)c1cccc2nc(C(F)F)ccc12] | N-[6-(2,2-difluoroethoxy)- 5-fluoro-2-methoxy-3- pyridyl]-2- (difluoromethyl)quinoline- 5-sulfonamide | A21 | B2 | 464.1 [M + H]⁺ |
| 36 | 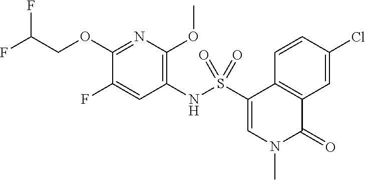 [SMILES: COc1nc(OCC(F)F)c(F)cc1NS(=O)(=O)c1cn(C)c(=O)c2cc(Cl)ccc12] | 7-chloro-N-[6-(2,2- difluoroethoxy)-5-fluoro-2- methoxy-3-pyridyl]-1-keto- 2-methyl-isoquinoline-4- sulfonamide | A23 | B2 | 478.2 [M + H]⁺ |
| 37 | 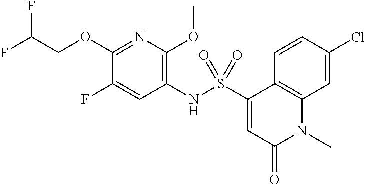 [SMILES: COc1nc(OCC(F)F)c(F)cc1NS(=O)(=O)c1cc(=O)n(C)c2cc(Cl)ccc12] | 7-chloro-N-[6-(2,2- difluoroethoxy)-5-fluoro-2- methoxy-3-pyridyl]-2-keto- 1-methyl-quinoline-4- sulfonamide | A24 | B2 | 476.3 [M + H]⁺ |
| 38 | 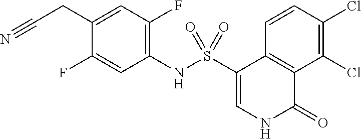 [SMILES: N#CCc1cc(F)c(NS(=O)(=O)c2c[nH]c(=O)c3c(Cl)c(Cl)ccc23)cc1F] | 7,8-dichloro-N-[4- (cyanomethyl)-2,5-difluoro- phenyl]-1-keto-2H- isoquinoline-4-sulfonamide | A25 | B1 | 444.1 [M + H]⁺ |
| 39 | 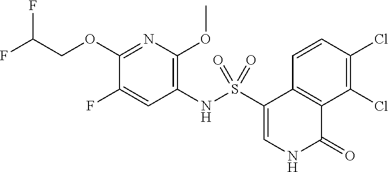 [SMILES: COc1nc(OCC(F)F)c(F)cc1NS(=O)(=O)c1c[nH]c(=O)c2c(Cl)c(Cl)ccc12] | 7,8-dichloro-N-[6-(2,2- difluroethoxy)-5-fluoro-2- methoxy-3-pyridyl]-1-keto- 2H-isoquinoline-4- sulfonamide | A25 | B2 | 498.1 [M + H]⁺ |

### Example 40: N-[6-(2,2-difluoroethoxy)-5-fluoro-2-methoxy-3-pyridyl]-5-(dimethylamino)naphthalene-1-sulfonamide

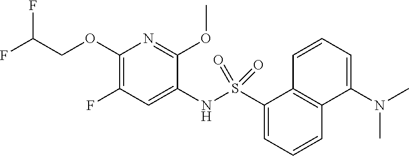 [SMILES: COc1nc(OCC(F)F)c(F)cc1NS(=O)(=O)c1cccc2c(N(C)C)cccc12]

A mixture of 6-(2,2-difluoroethoxy)-5-fluoro-2-methoxy-pyridin-3-amine (27 mg, 0.122 mmol, intermediate B2), 5-(dimethyl amino)naphthalene-1-sulfonyl chloride (36 mg, 0.134 mmol, intermediate A22) and pyridine (48 mg, 49 μL, 0.608 mmol) in dichloromethane (4 ml) was stirred at room temperature overnight. The reaction mixture was poured into water and extracted with dichloromethane twice. The combined organic layers were dried over Na2SO4, filtered and concentrated in vacuo. The crude material was purified by flash chromatography (silica gel, 0% to 50% ethyl acetate in heptane) to afford the title compound (30 mg, 540%) as a light-yellow solid, MS (ESI): m/z=456.3 [M+H]+.

The following Examples 41-46 were prepared in analogy to Example 40 by coupling the indicated sulfonylchloride intermediates A and amine intermediates B.

| | | | | | MS |
|---|---|---|---|---|---|
| | | | | | (ESI): |
| Ex. | Structure | Name | Int. A | Int. B | m/z |
| 41 | 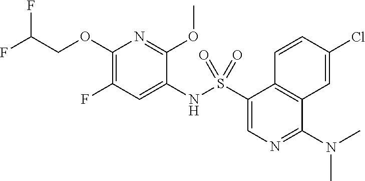 [SMILES: COc1nc(OCC(F)F)c(F)cc1NS(=O)(=O)c1cnc(N(C)C)c2cc(Cl)ccc12] | 7-chloro-N-[6-(2,2- difluoroethoxy)-5-fluoro-2- methoxy-3-pyridyl]-1- (dimethylamino)isoquinoline- 4-sulfonamide | A26 | B2 | 491.2 [M + H]⁺ |
| 42 | 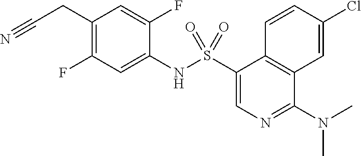 [SMILES: CN(C)c1ncc(S(=O)(=O)Nc2cc(F)c(CC#N)cc2F)c2ccc(Cl)cc12] | 7-chloro-N-[4- (cyanomethyl)-2,5-difluoro- phenyl]-1- (dimethylamino)isoquinoline- 4-sulfonamide | A26 | B1 | 437.2 [M + H]⁺ |
| 43 | 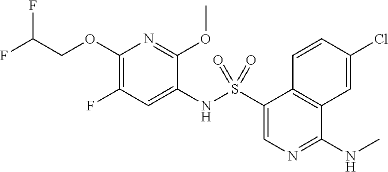 [SMILES: CNc1ncc(S(=O)(=O)Nc2cc(F)c(OCC(F)F)nc2OC)c2ccc(Cl)cc12] | 7-chloro-N-[6-(2,2- difluoroethoxy)-5-fluoro-2- methoxy-3-pyridyl]-1- (methylamino)isoquinoline- 4-sulfonamide | A16 | B2 | 477.2 [M + H]⁺ |
| 44 | 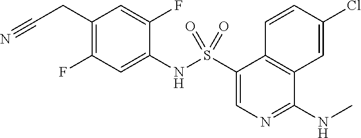 [SMILES: CNc1ncc(S(=O)(=O)Nc2cc(F)c(CC#N)cc2F)c2ccc(Cl)cc12] | 7-chloro-N-[4- (cyanomethyl)-2,5-difluoro- phenyl]-1- (methylamino)isoquinoline- 4-sulfonamide | A16 | B1 | 423.1 [M + H]⁺ |
| 45 | 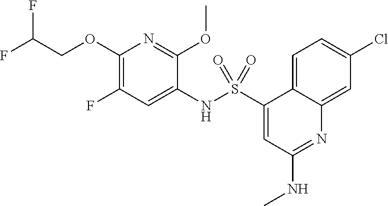 [SMILES: CNc1cc(S(=O)(=O)Nc2cc(F)c(OCC(F)F)nc2OC)c2ccc(Cl)cc2n1] | 7-chloro-N-[6-(2,2- difluoroethoxy)-5-fluoro-2- methoxy-3-pyridyl]-2- (methylamino)quinoline-4- sulfonamide | A17 | B2 | 477.2 [M + H]⁺ |
| 46 | 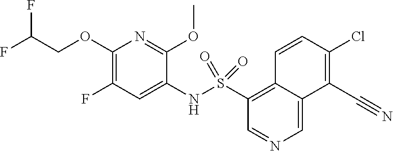 [SMILES: COc1nc(OCC(F)F)c(F)cc1NS(=O)(=O)c1cncc2c(C#N)c(Cl)ccc12] | 7-chloro-8-cyano-N-[6-(2,2- difluoroethoxy)-5-fluoro-2- methoxy-3- pyridyl] isoquinoline-4- sulfonamide | A20 | B2 | 473.1 [M + H]⁺ |
| 47 | 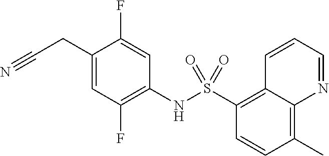 [SMILES: Cc1ccc(S(=O)(=O)Nc2cc(F)c(CC#N)cc2F)c2cccnc12] | N-[4-(cyanomethyl)-2,5- difluoro-phenyl]-8-methyl- quinoline-5-sulfonamide | A27 | B1 | 374.0 [M + H]⁺ |

### Example 48: 7-chloro-N-[6-(difluoromethoxy)-5-fluoro-2-methoxy-3-pyridyl]quinoline-4-sulfonamide

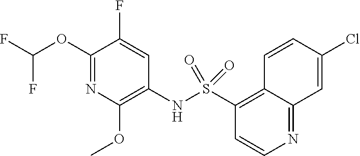 [SMILES: COc1nc(OC(F)F)c(F)cc1NS(=O)(=O)c1ccnc2cc(Cl)ccc12]

6-(Difluoromethoxy)-5-fluoro-2-methoxy-pyridin-3-amine (25.6 mg, 0.123 mmol, Intermediate B5) was dissolved in tetrahydrofuran (0.5 ml). At 0° C. under argon atmosphere lithium hexamethyldisilazide solution (1M in THF, 244 ul, 0.244 mmol) was added dropwise. The reaction mixture was stirred for 15 min. Then, a solution of 7-chloroquinoline-4-sulfonyl fluoride (30 mg, 0.122 mmol, Intermediate A2) in tetrahydrofuran (0.3 ml) was added dropwise. The mixture was stirred at room temperature for 3 h. Brine was added to the reaction mixture and it was extracted with ethyl acetate (3×). The combined organic layers were dried over Na2SO4, filtered and concentrated in vacuo. The residue was purified by preparative HPLC (column: YMC-Triart C18, 12 nm, 5 μm, 100×30 mm, gradient acetonitrile/water+0.1% HCOOH) to afford the title compound as red solid (18 mg, 32%), MS (ESI): m/z=434.1 [M+H]+.

### Example 49: 7-chloro-N-[4-(cyanomethyl)-2,5-difluoro-phenyl]-1-methoxy-isoquinoline-4-sulfonamide

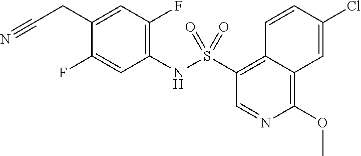 [SMILES: COc1ncc(S(=O)(=O)Nc2cc(F)c(CC#N)cc2F)c2ccc(Cl)cc12]

7-Chloro-1-methoxy-isoquinoline-4-sulfonyl chloride (64 mg, 0.22 mmol, Intermediate A19) was dissolved in pyridine (1 ml) and acetonitrile (1 ml). Then 2-(4-amino-2,5-difluoro-phenyl)acetonitrile (75 mg, 0.44 mmol, Intermediate B1) was added at room temperature and the mixture was stirred for 2 h, then the solvent was evaporated. The crude material was purified by preparative HPLC (column: SunFire C18 100×19 mm 5 um, gradient 40-90% 0.5-6.5 min water-acetonitrile) to afford the title compound (10 mg, 11% yield) as light yellow solid, MS (ESI): m/z=424.0 [M+H]+.

### Example 50: 2-chloro-N-[6-(2,2-difluoroethoxy)-5-fluoro-2-methoxy-3-pyridyl]-1,7-naphthyridine-5-sulfonamide

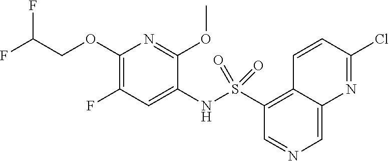 [SMILES: COc1nc(OCC(F)F)c(F)cc1NS(=O)(=O)c1cncc2nc(Cl)ccc12]

A 3 ml glass tube was charged with 2-chloro-1,7-naphthyridine-5-sulfonyl chloride (47 mg, 0.179 mmol, Intermediate A7), 6-(2,2-difluoroethoxy)-5-fluoro-2-methoxy-pyridin-3-amine (40 mg, 0.179 mmol, Intermediate B2), dichloromethane (0.7 ml) and N,N-diethyl isopropylamine (23 mg, 31 ul, 0.179 mmol) and the mixture was stirred at room temperature for 3 h. The mixture was partitioned between dichloromethane and water. The combined organic layers were dried over Na2SO4, filtered and concentrated under reduced pressure. The crude material was purified by preparative HPLC (column: Gemini NX, 12 nm, 5 μm, 100×30 mm, gradient acetonitrile/water+0.1% HCOOH) to afford the title compound as orange solid (1.5 mg, 1.7%), MS m/z 449.1 [M+H]+, ESI pos.

### Example 51: 2-chloro-N-[5-(difluoromethoxy)-4,6-dimethoxy-pyrimidin-2-yl]quinoline-5-sulfonamide

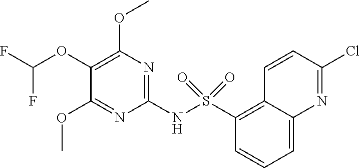 [SMILES: COc1nc(NS(=O)(=O)c2cccc3nc(Cl)ccc23)nc(OC)c1OC(F)F]

A solution of 5-(difluoromethoxy)-4,6-dimethoxy-pyrimidin-2-amine (7 mg, 0.025 mmol, Intermediate B8) in N,N-dimethylformamide (100 ul) was cooled to 0° C. Sodium hydride (60%, 1.2 mg, 0.030 mmol) was added and the reaction mixture was stirred at 0° C. for 10 min. 2-Chloroquinoline-5-sulfonyl chloride (8.0 mg, 0.030 mmol, Intermediate A3) was added. The reaction mixture was allowed to warm to room temperature and stirred for 10 min. The resulting suspension was carefully quenched with water, poured into brine and extracted twice with ethyl acetate. The organic layers were dried over Na2SO4, filtered and concentrated in vacuo. The crude material was purified by flash chromatography (silica gel, 0% to 40% ethyl acetate in heptane) to afford the title compound as white solid (5 mg, 43% yield). MS (ESI) m/z: 447.1 [M+H]+

### Example 52: N-(4-(cyanomethyl)-2,5-difluorophenyl)-1-oxo-1,2-dihydroisoquinoline-4-sulfonamide

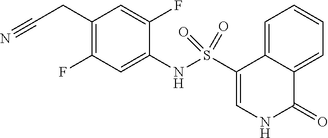 [SMILES: N#CCc1cc(F)c(NS(=O)(=O)c2c[nH]c(=O)c3ccccc23)cc1F]

To a stirred solution of 2-(4-amino-2,5-difluorophenyl)acetonitrile (25 mg, 0.149 mmol, Intermediate B1) and 4-dimethylamino pyridine (3.7 mg, 0.03 mmol) in pyridine (0.4 ml) was added 1-oxo-2H-isoquinoline-4-sulfonyl chloride (54.3 mg, 0.22 mmol, Intermediate A5). The reaction mixture was stirred at room temperature for 60 min, poured into dichloromethane and extracted with 10% citric acid solution. The organic layers were dried over Na2SO4 and concentrated in vacuo. The residue was purified by chromatography (silica gel, n-heptane/ethyl acetate 0 to 100%) to provide the title compound as a white solid (17 mg, 29% yield). MS (ESI) m/z: 376.0 [M+H]⁺

### Example 53: 7-chloro-N-[4-(cyanomethyl)-2,5-difluoro-phenyl]quinoline-4-sulfonamide

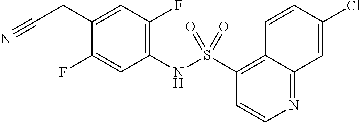 [SMILES: N#CCc1cc(F)c(NS(=O)(=O)c2ccnc3cc(Cl)ccc23)cc1F]

### Step 1: N-(4-bromo-2,5-difluoro-phenyl)-7-chloro-quinoline-4-sulfonamide

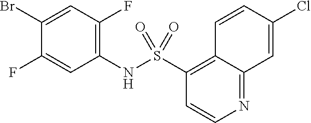 [SMILES: O=S(=O)(Nc1cc(F)c(Br)cc1F)c1ccnc2cc(Cl)ccc12]

A solution of lithium bis(trimethylsilyl)amide in tetrahydrofuran (1.71 ml, 1.71 mmol) was added dropwise to a solution of 4-bromo-2,5-difluoroaniline (169 mg, 0.81 mmol) in tetrahydrofuran (10 ml) at 0° C. under argon atmosphere. After 30 min a solution of 7-chloroquinoline-4-sulfonyl fluoride (200 mg, 0.81 mmol, Intermediate A2) in tetrahydrofuran (5 ml) was added dropwise and the resulting mixture was stirred at room temperature for 12 h. The resulting mixture was poured into brine and extracted with ethyl acetate (2×30 ml). The combined organic layers were evaporated to obtain crude N-(4-bromo-2,5-difluoro-phenyl)-7-chloro-quinoline-4-sulfonamide (200 mg, 0.48 mmol, 59% yield), which was used in next step without purification.

### Step 2: 7-chloro-N-[4-(cyanomethyl)-2,5-difluoro-phenyl]quinoline-4-sulfonamide

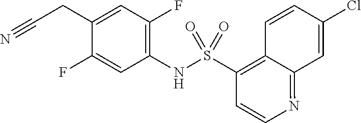 [SMILES: N#CCc1cc(F)c(NS(=O)(=O)c2ccnc3cc(Cl)ccc23)cc1F]

N-(4-bromo-2,5-difluoro-phenyl)-7-chloro-quinoline-4-sulfonamide (200 mg, 0.46 mmol) was dissolved in dimethylsulfoxide (6 ml) and water (1 ml), then with potassium fluoride (80.4 mg, 1.38 mmol) and 4-isoxazoleboronic acid pinacol ester (180 mg, 0.92 mmol) were added. The resulting mixture was evacuated and backfilled with argon for 3 times. 1,1′-bis(diphenylphosphino)ferrocene-palladium(II)dichloride dichloromethane complex (18.8 mg, 0.02 mmol) was added and the resulting mixture was heated to 120° C. for 48 h, then cooled to room temperature and filtered. The filtrate was subjected to HPLC (column: SunFire C18 100×19 mm Sum, 4% water-acetonitrile+0.1% NH4OH) to obtain the title compound as brown gum (5 mg, 3% yield), MS (ESI) m/z: 394.0 [M+H]⁺

### Example 54: 2-bromo-N-[6-(2,2-difluoroethoxy)-5-fluoro-2-methoxy-3-pyridyl]quinoline-5-sulfonamide

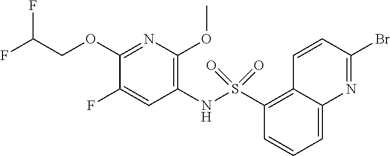 [SMILES: COc1nc(OCC(F)F)c(F)cc1NS(=O)(=O)c1cccc2nc(Br)ccc12]

To a solution of 2-chloro-N-[6-(2,2-difluoroethoxy)-5-fluoro-2-methoxy-3-pyridyl]quinoline-5-sulfonamide (50 mg, 0.112 mmol, Example 10) in propionitrile (1.2 ml) was added bromotrimethylsilane (34 mg, 30 ul, 0.22 mmol) and the mixture was stirred overnight. Then it was poured onto mixture of 2N sodium hydroxide and ice, and extracted with ethyl acetate twice. The combined organic layers were dried over Na2SO4, filtered and concentrated in vacuo. The yellow residue was purified by flash chromatography (silica gel, 0 to 100% ethyl acetate in n-heptane) to afford the title compound as red solid. (19.6 mg, 32%), MS (ESI) m/z: 494.1/492.0 [M+H]⁺

### Example 55: 2-cyclopropyl-N-[6-(2,2-difluoroethoxy)-5-fluoro-2-methoxy-3-pyridyl]quinoline-5-sulfonamide

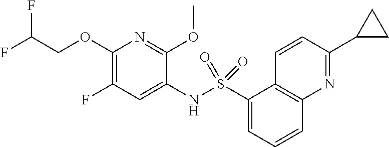 [SMILES: COc1nc(OCC(F)F)c(F)cc1NS(=O)(=O)c1cccc2nc(C3CC3)ccc12]

In a reaction tube, 2-chloro-N-[6-(2,2-difluoroethoxy)-5-fluoro-2-methoxy-3-pyridyl]quinoline-5-sulfonamide (50 mg, 0.112 mmol, Example 10), cyclopropylboronic acid (19 mg, 0.22 mmol), toluene (0.44 ml) and water (0.022 mL) were added and the mixture was purged with argon in ultrasonic bath for 5 min. Then, tricyclohexyl phosphine (6.3 mg, 0.022 mmol), palladium(II) acetate (2.5 mg, 0.011 mmol) and tripotassium phosphate (83 mg, 0.39 mmol) were added. The reaction mixture was stirred at 110° C. for 19 h, then concentrated in vacuo. To the residue was added dichloromethane and the insoluble parts were filtered off. The filtrate was concentrated in vacuo and the residue was purified by flash chromatography (silica gel, 0% to 30% ethyl acetate in n-heptane) to afford the title compound as off-white solid (2.5 mg, 5%), MS m/z=454.3 (M+H)⁺

### Example 56: N-[6-(2,2-difluoroethoxy)-5-fluoro-2-methoxy-3-pyridyl]-2-methoxy-quinoline-5-sulfonamide

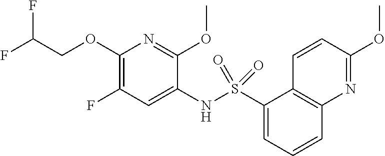 [SMILES: COc1ccc2c(S(=O)(=O)Nc3cc(F)c(OCC(F)F)nc3OC)cccc2n1]

To a solution of 2-chloro-N-[6-(2,2-difluoroethoxy)-5-fluoro-2-methoxy-3-pyridyl]quinoline-5-sulfonamide (5 mg, 0.011 mmol, Example 10) in methanol (0.06 ml) under nitrogen at room temperature, sodium methoxide (1.9 mg, 0.035 mmol) was added. The reaction mixture was stirred at 100° C. for 19 h. The mixture was cooled to room temperature and the methanol was evaporated. The residue was extracted with ethyl acetate twice, and washed with water. The combined organic layers were dried over Na2SO4, filtered and evaporated. The crude material was purified by flash chromatography (silica gel, ethyl acetate in heptane 0% to 30%) to give the title compound as off-white solid (5 mg, 98%), MS: m/z=444.1 (M+H)⁺

### Example 57: N-[6-(2,2-difluoroethoxy)-5-fluoro-2-methoxy-3-pyridyl]-1-(dimethylamino)isoquinoline-5-sulfonamide

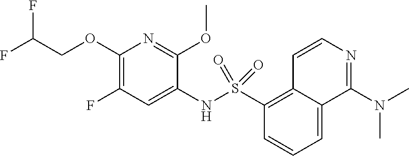 [SMILES: COc1nc(OCC(F)F)c(F)cc1NS(=O)(=O)c1cccc2c(N(C)C)nccc12]

### Step 1: 1-chloro-N-[6-(2,2-difluoroethoxy)-5-fluoro-2-methoxy-3-pyridyl]isoquinoline-5-sulfonamide

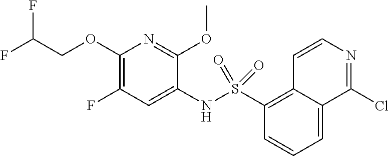 [SMILES: COc1nc(OCC(F)F)c(F)cc1NS(=O)(=O)c1cccc2c(Cl)nccc12]

To a solution of 1-chloroisoquinoline-5-sulfonyl chloride (CAS 141519-77-9, 50 mg, 0.19 mmol) in dichloromethane (1 ml) was added pyridine (0.05 ml, 0.57 mmol) and 6-(2,2-difluoroethoxy)-5-fluoro-2-methoxy-pyridin-3-amine (42 mg, 0.19 mmol, Intermediate B2). The mixture was stirred at 25° C. for 12 h. The mixture was concentrated under reduced pressure to give the crude material which was purified by column chromatography (petroleum ether-ethyl acetate 1:0 to 0:1) to give 1-chloro-N-[6-(2,2-difluoroethoxy)-5-fluoro-2-methoxy-3-pyridyl]isoquinoline-5-sulfonamide as a yellow solid (89 mg, 99% yield), MS m/z=448.0 [M+H]⁺ (ESI+).

### Step 2: N-[6-(2,2-difluoroethoxy)-5-fluoro-2-methoxy-3-pyridyl]-1-(dimethylamino)isoquinoline-5-sulfonamide

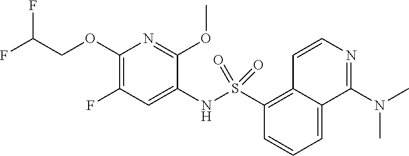 [SMILES: COc1nc(OCC(F)F)c(F)cc1NS(=O)(=O)c1cccc2c(N(C)C)nccc12]

A solution of 1-chloro-N-[6-(2,2-difluoroethoxy)-5-fluoro-2-methoxy-3-pyridyl]isoquinoline-5-sulfonamide (80.0 mg, 0.18 mmol) in dimethylamine solution (30% in ethanol, 3 ml) was stirred at 60° C. for 12 h. The mixture was concentrated under reduced pressure to give the crude material which was purified by preparative HPLC (column: Phenomenex Synergi C18 150*25 mm*10 um, water, 0.2% TFA), acetonitrile) to give the title compound as a light red solid (74.5 mg, 91% yield), MS m/z=457.1, [M+H]⁺ (ESI pos.)

### Example 58: N-[6-(2,2-difluoroethoxy)-5-fluoro-2-methoxy-3-pyridyl]tetralin-5-sulfonamide

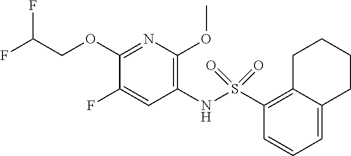 [SMILES: COc1nc(OCC(F)F)c(F)cc1NS(=O)(=O)c1cccc2c1CCCC2]

The title compound was prepared in analogy to Example 1 using tetralin-5-sulfonyl chloride (Intermediate A28) instead of naphthalene-1-sulfonyl chloride and 6-(2,2-difluoroethoxy)-5-fluoro-2-methoxy-pyridin-3-amine (Intermediate B2) instead of 2-(4-amino-2,5-difluoro-phenyl)acetonitrile as white solid, MS m/z=417.2 [M+H]⁺ (ESI pos.).

### Example A

A compound of formula I can be used in a manner known per se as the active ingredient for the production of tablets of the following composition:

| | Per tablet | |
|---|---|---|
| Active ingredient | 200 | mg |
| Microcrystalline cellulose | 155 | mg |
| Corn starch | 25 | mg |
| Talc | 25 | mg |
| Hydroxypropylmethylcellulose | 20 | mg |
| | 425 | mg |

### Example B

A compound of formula I can be used in a manner known per se as the active ingredient for the production of capsules of the following composition:

| | Per capsule | |
|---|---|---|
| Active ingredient | 100.0 | mg |
| Corn starch | 20.0 | mg |
| Lactose | 95.0 | mg |
| Talc | 4.5 | mg |
| Magnesium stearate | 0.5 | mg |
| | 220.0 | mg |

1. Compounds of formula I 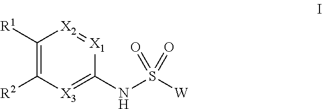
wherein,
R¹ is selected from cyanoalkyl, haloalkoxy, haloalkyl, cyclopropyl and oxetanyl;
R² is selected from alkoxy, H or halo;
X₁ is N and X₂ is CR⁴ and X₃ is N, or
X₁ is CR³, X₂ is CR⁴, and X₃ is N or CR⁵, or
X₁ is CR³, X₂ is N, and X₃ is CR⁵;
R³ is selected from alkoxy, H, halo, haloalkoxy;
R⁴ is selected from alkoxy, H, halo;
R⁵ is selected from H, halo;
W is selected from Ring A, Ring B, or Ring C 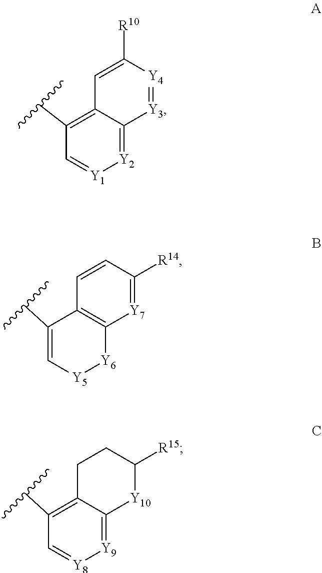
Y₁ is CR⁶, Y₂ is CR⁷, Y₃ is CR⁸ and Y₄ is CR⁹, or
Y₁ is N, Y₂ is CR⁷, Y₃ is CR⁸ and Y₄ is CR⁹, or
Y₁ is N, Y₂ is CR⁷, Y₃ is N and Y₄ is CR⁹, or
Y₁ is CR⁶, Y₂ is N, Y₃ is CR⁸ and Y₄ is CR⁹, or
Y₁ is CR⁶, Y₂ is CR⁷, Y₃ is N and Y₄ is CR⁹, or
Y₁ is CR⁶, Y₂ is CR⁷, Y₃ is CR⁸ and Y₄ is N;
R⁶ is H or alkylamino;
R⁷ is alkoxy, dimethylamino, H, methyl, methylamino, OH;
R⁸ is cyano, dimethylamino, H, halo;
R⁹ is alkoxy, cyclopropyl, haloalkyl dimethylamino, H, halo, alkyl;
R¹⁰ is H or halo;
Y₅ is NR¹¹, Y₆ is —C(═O)—, and Y₇ is CR¹³ or N, or
Y₅ is —C(═O)—, Y₆ is NR¹² and Y₇ is CR¹³;
R¹¹ is alkyl or H;
R¹² is alkyl or H;
R¹³ is H, alkyl or halo;
R¹⁴ is H, alkyl, halo, or haloalkyl;
Y₈ is —CH— or —N—;
Y₉ is —CH— or —N—;
Y₁₀ is —CH₂— or —O—;
R¹⁵ is H, alkyl, or haloalkyl;
and pharmaceutically acceptable salts thereof.

2. A compound according to claim 1, wherein R¹ is cyanoalkyl, haloalkoxy, or cyclopropyl.

3. A compound according to claim 1, wherein R² is alkoxy or halo.

4. A compound according to claim 1, wherein R³ is alkoxy, halo, or haloalkoxy.

5. A compound according to claim 1, wherein X₁ is CR³, X₂ is CR⁴, and X₃ is N
or CR⁵, or X₁ is CR3, X₂ is N, and X₃ is CR⁵.

6. A compound according to claim 1, wherein R⁴ and R⁵ are H.

7. A compound according to claim 1, wherein
Y₁ is CR⁶, Y₂ is CR⁷, Y₃ is CR⁸ and Y₄ is CR⁹, or
Y₁ is N, Y₂ is CR⁷, Y₃ is CR⁸ and Y₄ is CR⁹, or
Y₁ is N, Y₂ is CR⁷, Y₃ is N and Y₄ is CR⁹, or
Y₁ is CR⁶, Y₂ is CR⁷, Y₃ is N and Y₄ is CR⁹.

8. A compound according to claim 1, wherein R⁶ is H.

9. A compound according to claim 1, wherein R⁷ is H or methylamino.

10. A compound according to claim 1, wherein R⁸ is dimethylamino, H or halo.

11. A compound according to claim 1, wherein R⁹ is cyclopropyl, H, halo or alkyl.

12. A compound according to claim 1, wherein R¹⁰, R¹¹ and R¹² are H.

13. A compound according to claim 1, wherein R¹³ is H or halo.

14. A compound according to claim 1, wherein R¹⁴ is H or halo.

15. A compound according to claim 1, wherein R¹⁴ is halo.

16. A compound according to claim 1, wherein Y₈ is —CH—, Y₉ is —CH— and Y₁₀ is —CH— or —O—.

17. A compound according to claim 1, wherein Y₈ is —CH—, Y₉ is —CH— and Y₁₀
is —O—.

18. A compound according to claim 1, wherein R¹⁵ is H or alkyl.

19. A compound according to claim 1, wherein R¹⁵ is alkyl.

20. A compound according to claim 1, wherein
when R² is H, then X₂ is CR⁴ and R⁴ is not H;
when R¹ is haloalkyl, then R² is alkoxy; and
when R¹ is haloalkoxy and R² is halo, then X₂ is N.

21. A compound according to claim 1, wherein
R¹ is selected from cyanoalkyl, haloalkoxy, haloalkyl, cyclopropyl and oxetanyl;
R² is selected from alkoxy, H or halo;
X₁ is N and X₂ is CR⁴ and X₃ is N, or
X₁ is CR³, X₂ is CR⁴, and X₃ is N or CR⁵, or
X₁ is CR³, X₂ is N, and X₃ is CR⁵;
R³ is selected from alkoxy, H, halo, haloalkoxy;
R⁴ is selected from alkoxy, H, halo;
R⁵ is selected from H, halo;
W is selected from Ring A, Ring B, or Ring C 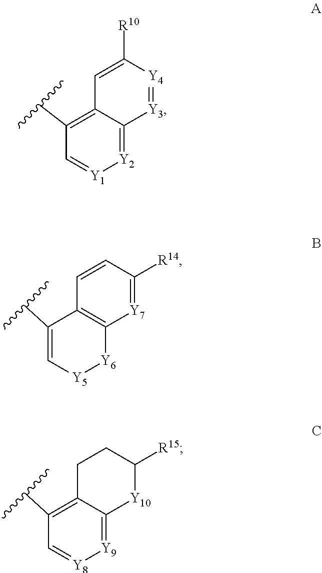
Y₁ is CR⁶, Y₂ is CR⁷, Y₃ is CR⁸ and Y₄ is CR⁹, or
Y₁ is N, Y₂ is CR⁷, Y₃ is CR⁸ and Y₄ is CR⁹, or
Y₁ is N, Y₂ is CR⁷, Y₃ is N and Y₄ is CR⁹, or
Y₁ is CR⁶, Y₂ is N, Y₃ is CR⁸ and Y₄ is CR⁹, or
Y₁ is CR⁶, Y₂ is CR⁷, Y₃ is N and Y₄ is CR⁹, or
Y₁ is CR⁶, Y₂ is CR⁷, Y₃ is CR⁸ and Y₄ is N;
R⁶ is H or alkylamino;
R⁷ is alkoxy, dimethylamino, H, methyl, methylamino, OH;
R⁸ is cyano, dimethylamino, H, halo;
R⁹ is alkoxy, cyclopropyl, haloalkyl dimethylamino, H, halo, alkyl;
R¹⁰ is H or halo;
Y₅ is NR¹¹, Y₆ is —C(═O)—, and Y₇ is CR¹³ or N, or
Y₅ is —C(═O)—, Y₆ is NR¹² and Y₇ is CR¹³;
R¹¹ is alkyl or H;
R¹² is alkyl or H;
R¹³ is H, or halo:
R¹⁴ is H or halo:
Y₈ is —CH—;
Y₉ is —CH—;
Y₁₀ is —CH₂— or —O—;
R¹⁵ is H, or alkyl;
and pharmaceutically acceptable salts thereof.

22. A compound according to claim 1, wherein
R¹ is selected from cyanoalkyl, haloalkoxy, haloalkyl, cyclopropyl and oxetanyl;
R² is selected from alkoxy, H or halo;
X₁ is N and X₂ is CR⁴ and X₃ is N, or
X₁ is CR³, X₂ is CR⁴, and X₃ is N or CR⁵, or
X₁ is CR³, X₂ is N, and X₃ is CR⁵;
R³ is selected from alkoxy, H, halo, haloalkoxy;
R⁴ is selected from alkoxy, H, halo;
R⁵ is selected from H, halo;
W is selected from Ring A, Ring B, or Ring C 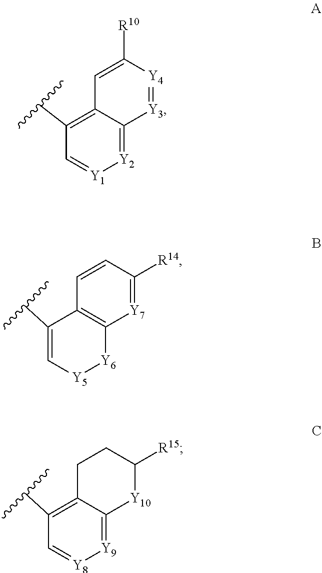
Y₁ is CR⁶, Y₂ is CR⁷, Y₃ is CR⁸ and Y₄ is CR⁹, or
Y₁ is N, Y₂ is CR⁷, Y₃ is CR⁸ and Y₄ is CR⁹, or
Y₁ is N, Y₂ is CR⁷, Y₃ is N and Y₄ is CR⁹, or
Y₁ is CR⁶, Y₂ is N, Y₃ is CR⁸ and Y₄ is CR⁹, or
Y₁ is CR⁶, Y₂ is CR⁷, Y₃ is N and Y₄ is CR⁹, or
Y₁ is CR⁶, Y₂ is CR⁷, Y₃ is CR⁸ and Y₄ is N;
R⁶ is H or alkylamino;
R⁷ is alkoxy, dimethylamino, H, methyl, methylamino, OH;
R⁸ is cyano, dimethylamino, H, halo;
R⁹ is alkoxy, cyclopropyl, haloalkyl dimethylamino, H, halo, alkyl;
R¹⁰ is H or halo;
Y₅ is NR¹¹, Y₆ is —C(═O)—, and Y₇ is CR¹³ or N, or
Y₅ is —C(═O)—, Y₆ is NR¹² and Y₇ is CR¹³;
R¹¹ is alkyl or H;
R¹² is alkyl or H;
R¹³ is H, or halo:
R¹⁴ is H or halo:
Y₈ is —CH—;
Y₉ is —CH—;
Y₁₀ is —CH₂— or —O—;
R¹⁵ is H, or alkyl;
wherein,
when R² is H, then X₂ is CR⁴ and R⁴ is not H;
when R¹ is haloalkyl, then R² is alkoxy;
when R¹ is haloalkoxy and R² is halo, then X₂ is N;
and pharmaceutically acceptable salts thereof.

23. A compound according to claim 1, wherein
R¹ is selected from cyanoalkyl, haloalkoxy, or cyclopropyl;
R² is selected from alkoxy or halo;
X₁ is CR³, X₂ is CR⁴, and X₃ is N or CR⁵, or
X₁ is CR³, X₂ is N, and X₃ is CR⁵.
R³ is selected from alkoxy, halo, or haloalkoxy;
R⁴ is H;
R⁵ is H;
W is selected from Ring A, Ring B, or Ring C 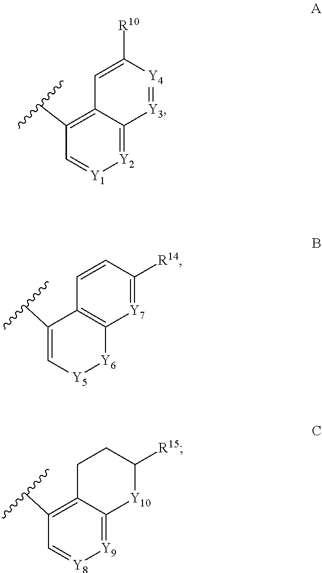
Y₁ is CR⁶, Y₂ is CR⁷, Y₃ is CR⁸ and Y₄ is CR⁹, or
Y₁ is N, Y₂ is CR⁷, Y₃ is CR⁸ and Y₄ is CR⁹, or
Y₁ is N, Y₂ is CR⁷, Y₃ is N and Y₄ is CR⁹, or
Y₁ is CR⁶, Y₂ is CR⁷, Y₃ is N and Y₄ is CR⁹.
R⁶ is H;
R⁷ is H or methylamino;
R⁸ is dimethylamino, H or halo;
R⁹ is cyclopropyl, H, halo or alkyl;
R¹⁰ is H;
Y₅ is NR¹¹, Y₆ is —C(═O)—, and Y₇ is CR¹³ or N, or
Y₅ is —C(═O)—, Y₆ is NR¹² and Y₇ is CR¹³;
R¹¹ is H;
R¹² is;
R¹³ is H, or halo:
R¹⁴ is halo:
Y₈ is —CH—;
Y₉ is —CH—;
Y₁₀ is —O—;
R¹⁵ is alkyl;
and pharmaceutically acceptable salts thereof.

24. A compound according to claim 1, wherein
R¹ is selected from cyanoalkyl, haloalkoxy, or cyclopropyl;
R² is selected from alkoxy or halo;
X₁ is CR³, X₂ is CR⁴, and X₃ is N or CR⁵, or
X₁ is CR³, X₂ is N, and X₃ is CR⁵.
R³ is selected from alkoxy, halo, or haloalkoxy;
R⁴ is H;
R⁵ is H;
W is selected from Ring A, Ring B, or Ring C 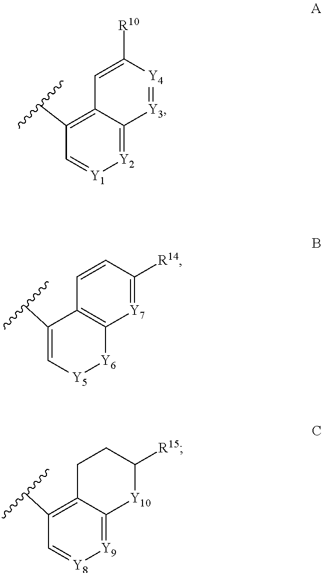
Y₁ is CR⁶, Y₂ is CR⁷, Y₃ is CR⁸ and Y₄ is CR⁹, or
Y₁ is N, Y₂ is CR⁷, Y₃ is CR⁸ and Y₄ is CR⁹, or
Y₁ is N, Y₂ is CR⁷, Y₃ is N and Y₄ is CR⁹, or
Y₁ is CR⁶, Y₂ is CR⁷, Y₃ is N and Y₄ is CR⁹.
R⁶ is H;
R⁷ is H or methylamino;
R⁸ is dimethylamino, H or halo;
R⁹ is cyclopropyl, H, halo or alkyl;
R¹⁰ is H;
Y₅ is NR¹¹, Y₆ is —C(═O)—, and Y₇ is CR¹³ or N, or
Y₅ is —C(═O)—, Y₆ is NR¹² and Y₇ is CR¹³;
R¹¹ is H;
R¹² is;
R¹³ is H, or halo:
R¹⁴ is halo.
Y₅ is —CH—;
Y₉ is —CH—;
Y₁₀ is —O—;
R¹⁵ is alkyl;
wherein,
when R² is H, then X₂ is CR⁴ and R⁴ is not H;
when R¹ is haloalkyl, then R² is alkoxy;
when R¹ is haloalkoxy and R² is halo, then X₂ is N;
and pharmaceutically acceptable salts thereof.

25. A compound according to claim 1, selected from
N-(4-(cyanomethyl)-2,5-difluorophenyl)naphthalene-1-sulfonamide;
N-[6-(2,2-difluoroethoxy)-5-fluoro-2-methoxy-3-pyridyl]naphthalene-1-sulfonamide;
2-chloro-N-[4-(cyanomethyl)-2,5-difluoro-phenyl]quinoline-5-sulfonamide;
2-chloro-N-(6-cyclopropyl-5-fluoro-2-methoxy-3-pyridyl)quinoline-5-sulfonamide;
2-chloro-N-[6-(cyanomethyl)-5-fluoro-2-methoxy-3-pyridyl]quinoline-5-sulfonamide;
2-chloro-N-[6-(difluoromethoxy)-5-fluoro-2-methoxy-3-pyridyl]quinoline-5-sulfonamide;
2-chloro-N-[5-fluoro-2-methoxy-6-(oxetan-3-yl)-3-pyridyl]quinoline-5-sulfonamide;
2-chloro-N-[5-(2,2-difluoroethyl)-4,6-dimethoxy-pyrimidin-2-yl]quinoline-5-sulfonamide;
2-chloro-N-[5-(2,2-difluoroethoxy)-3-fluoro-6-methoxy-2-pyridyl]quinoline-5-sulfonamide;
2-chloro-N-[6-(2,2-difluoroethoxy)-5-fluoro-2-methoxy-3-pyridyl]quinoline-5-sulfonamide;
N-[2,6-bis(difluoromethoxy)-5-fluoro-3-pyridyl]-2-chloro-quinoline-5-sulfonamide;
7-chloro-N-[4-(cyanomethyl)-2,5-difluoro-phenyl]isoquinoline-4-sulfonamide;
7-chloro-N-[6-(difluoromethoxy)-5-fluoro-2-methoxy-3-pyridyl]isoquinoline-4-sulfonamide;
7-chloro-N-[5-(2,2-difluoroethoxy)-3-fluoro-6-methoxy-2-pyridyl]isoquinoline-4-sulfonamide;
7-chloro-N-[6-(2,2-difluoroethoxy)-5-fluoro-2-methoxy-3-pyridyl]isoquinoline-4-sulfonamide;
N-[6-(2,2-difluoroethoxy)-5-fluoro-2-methoxy-3-pyridyl]-2-methyl-chromane-5-sulfonamide;
5-chloro-N-[4-(cyanomethyl)-2,5-difluoro-phenyl]naphthalene-1-sulfonamide;
5-chloro-N-[6-(2,2-difluoroethoxy)-5-fluoro-2-methoxy-3-pyridyl]naphthalene-1-sulfonamide;
7-chloro-N-[6-(2,2-difluoroethoxy)-5-fluoro-2-methoxy-3-pyridyl]naphthalene-1-sulfonamide;
8-chloro-N-[4-(cyanomethyl)-2,5-difluoro-phenyl]-1-hydroxy-isoquinoline-4-sulfonamide;
7-chloro-N-[4-(cyanomethyl)-2,5-difluoro-phenyl]-1-keto-2H-isoquinoline-4-sulfonamide;
7-chloro-N-[5-(2-fluoroethoxy)-4-methoxy-pyrimidin-2-yl]-1-keto-2H-isoquinoline-4-sulfonamide;
7-chloro-N-[6-(difluoromethoxy)-5-fluoro-2-methoxy-3-pyridyl]-1-keto-2H-isoquinoline-4-sulfonamide;
7-chloro-N-[5-(2,2-difluoroethyl)-4,6-dimethoxy-pyrimidin-2-yl]-1-keto-2H-isoquinoline-4-sulfonamide;
7-chloro-N-[5-(2,2-difluoroethoxy)-3-fluoro-6-methoxy-2-pyridyl]-1-oxo-2H-isoquinoline-4-sulfonamide;
7-chloro-N-[4-(2,2-difluoroethoxy)-5-fluoro-2-methoxy-phenyl]-1-keto-2H-isoquinoline-4-sulfonamide;
N-[2,6-bis(difluoromethoxy)-5-fluoro-3-pyridyl]-7-chloro-1-keto-2H-isoquinoline-4-sulfonamide;
2-chloro-N-[4-(cyanomethyl)-2,5-difluoro-phenyl]-8-oxo-7H-1,7-naphthyridine-5-sulfonamide;
2-chloro-N-[6-(2,2-difluoroethoxy)-5-fluoro-2-methoxy-3-pyridyl]-8-keto-7H-1,7-naphthyridine-5-sulfonamide;
7-chloro-N-[6-(2,2-difluoroethoxy)-5-fluoro-2-methoxy-3-pyridyl]-2-keto-1H-quinoline-4-sulfonamide;
N-[6-(2,2-difluoroethoxy)-5-fluoro-2-methoxy-3-pyridyl]-2-(dimethylamino)quinoline-5-sulfonamide;
N-[4-(cyanomethyl)-2,5-difluoro-phenyl]-2-(dimethylamino)quinoline-5-sulfonamide;
N-[6-(2,2-difluoroethoxy)-5-fluoro-2-methoxy-3-pyridyl]-2-methyl-quinoline-5-sulfonamide;
6-chloro-N-[6-(2,2-difluoroethoxy)-5-fluoro-2-methoxy-3-pyridyl]naphthalene-1-sulfonamide;
N-[6-(2,2-difluoroethoxy)-5-fluoro-2-methoxy-3-pyridyl]-2-(difluoromethyl)quinoline-5-sulfonamide;
7-chloro-N-[6-(2,2-difluoroethoxy)-5-fluoro-2-methoxy-3-pyridyl]-1-keto-2-methyl-isoquinoline-4-sulfonamide;
7-chloro-N-[6-(2,2-difluoroethoxy)-5-fluoro-2-methoxy-3-pyridyl]-2-keto-1-methyl-quinoline-4-sulfonamide;
7,8-dichloro-N-[4-(cyanomethyl)-2,5-difluoro-phenyl]-1-keto-2H-isoquinoline-4-sulfonamide;
7,8-dichloro-N-[6-(2,2-difluoroethoxy)-5-fluoro-2-methoxy-3-pyridyl]-1-keto-2H-isoquinoline-4-sulfonamide;
N-[6-(2,2-difluoroethoxy)-5-fluoro-2-methoxy-3-pyridyl]-5-(dimethylamino)naphthalene-1-sulfonamide;
7-chloro-N-[6-(2,2-difluoroethoxy)-5-fluoro-2-methoxy-3-pyridyl]-1-(dimethylamino)isoquinoline-4-sulfonamide;
7-chloro-N-[4-(cyanomethyl)-2,5-difluoro-phenyl]-1-(dimethylamino)isoquinoline-4-sulfonamide;
7-chloro-N-[6-(2,2-difluoroethoxy)-5-fluoro-2-methoxy-3-pyridyl]-1-(methylamino)isoquinoline-4-sulfonamide;
7-chloro-N-[4-(cyanomethyl)-2,5-difluoro-phenyl]-1-(methylamino)isoquinoline-4-sulfonamide;
7-chloro-N-[6-(2,2-difluoroethoxy)-5-fluoro-2-methoxy-3-pyridyl]-2-(methylamino)quinoline-4-sulfonamide;
7-chloro-8-cyano-N-[6-(2,2-difluoroethoxy)-5-fluoro-2-methoxy-3-pyridyl]isoquinoline-4-sulfonamide;
7-chloro-N-[6-(difluoromethoxy)-5-fluoro-2-methoxy-3-pyridyl]quinoline-4-sulfonamide;
7-chloro-N-[4-(cyanomethyl)-2,5-difluoro-phenyl]-1-methoxy-isoquinoline-4-sulfonamide;
2-chloro-N-[6-(2,2-difluoroethoxy)-5-fluoro-2-methoxy-3-pyridyl]-1,7-naphthyridine-5-sulfonamide;
2-chloro-N-[5-(difluoromethoxy)-4,6-dimethoxy-pyrimidin-2-yl]quinoline-5-sulfonamide;
N-(4-(cyanomethyl)-2,5-difluorophenyl)-1-oxo-1,2-dihydroisoquinoline-4-sulfonamide;
7-chloro-N-[4-(cyanomethyl)-2,5-difluoro-phenyl]quinoline-4-sulfonamide;
2-bromo-N-[6-(2,2-difluoroethoxy)-5-fluoro-2-methoxy-3-pyridyl]quinoline-5-sulfonamide;
2-cyclopropyl-N-[6-(2,2-difluoroethoxy)-5-fluoro-2-methoxy-3-pyridyl]quinoline-5-sulfonamide;
N-[6-(2,2-difluoroethoxy)-5-fluoro-2-methoxy-3-pyridyl]-2-methoxy-quinoline-5-sulfonamide;
N-[6-(2,2-difluoroethoxy)-5-fluoro-2-methoxy-3-pyridyl]-1-(dimethylamino)isoquinoline-5-sulfonamide;
N-[6-(2,2-difluoroethoxy)-5-fluoro-2-methoxy-3-pyridyl]tetralin-5-sulfonamide;
and pharmaceutically acceptable salts thereof.

26. A compound according to claim 1, selected from
2-chloro-N-(6-cyclopropyl-5-fluoro-2-methoxy-3-pyridyl)quinoline-5-sulfonamide;
2-chloro-N-[6-(cyanomethyl)-5-fluoro-2-methoxy-3-pyridyl]quinoline-5-sulfonamide;
2-chloro-N-[6-(difluoromethoxy)-5-fluoro-2-methoxy-3-pyridyl]quinoline-5-sulfonamide;
2-chloro-N-[6-(2,2-difluoroethoxy)-5-fluoro-2-methoxy-3-pyridyl]quinoline-5-sulfonamide;
7-chloro-N-[6-(2,2-difluoroethoxy)-5-fluoro-2-methoxy-3-pyridyl]isoquinoline-4-sulfonamide;
N-[6-(2,2-difluoroethoxy)-5-fluoro-2-methoxy-3-pyridyl]-2-methyl-chromane-5-sulfonamide;
5-chloro-N-[6-(2,2-difluoroethoxy)-5-fluoro-2-methoxy-3-pyridyl]naphthalene-1-sulfonamide;
7-chloro-N-[6-(difluoromethoxy)-5-fluoro-2-methoxy-3-pyridyl]-1-keto-2H-isoquinoline-4-sulfonamide;
7-chloro-N-[5-(2,2-difluoroethoxy)-3-fluoro-6-methoxy-2-pyridyl]-1-oxo-2H-isoquinoline-4-sulfonamide;
7-chloro-N-[4-(2,2-difluoroethoxy)-5-fluoro-2-methoxy-phenyl]-1-keto-2H-isoquinoline-4-sulfonamide;
N-[2,6-bis(difluoromethoxy)-5-fluoro-3-pyridyl]-7-chloro-1-keto-2H-isoquinoline-4-sulfonamide;
2-chloro-N-[6-(2,2-difluoroethoxy)-5-fluoro-2-methoxy-3-pyridyl]-8-keto-7H-1,7-naphthyridine-5-sulfonamide;
7-chloro-N-[6-(2,2-difluoroethoxy)-5-fluoro-2-methoxy-3-pyridyl]-2-keto-1H-quinoline-4-sulfonamide;
N-[6-(2,2-difluoroethoxy)-5-fluoro-2-methoxy-3-pyridyl]-2-methyl-quinoline-5-sulfonamide;
6-chloro-N-[6-(2,2-difluoroethoxy)-5-fluoro-2-methoxy-3-pyridyl]naphthalene-1-sulfonamide;
7,8-dichloro-N-[6-(2,2-difluoroethoxy)-5-fluoro-2-methoxy-3-pyridyl]-1-keto-2H-isoquinoline-4-sulfonamide;
N-[6-(2,2-difluoroethoxy)-5-fluoro-2-methoxy-3-pyridyl]-5-(dimethylamino)naphthalene-1-sulfonamide;
7-chloro-N-[6-(2,2-difluoroethoxy)-5-fluoro-2-methoxy-3-pyridyl]-1-(methylamino)isoquinoline-4-sulfonamide;
2-chloro-N-[6-(2,2-difluoroethoxy)-5-fluoro-2-methoxy-3-pyridyl]-1,7-naphthyridine-5-sulfonamide;
2-bromo-N-[6-(2,2-difluoroethoxy)-5-fluoro-2-methoxy-3-pyridyl]quinoline-5-sulfonamide;
2-cyclopropyl-N-[6-(2,2-difluoroethoxy)-5-fluoro-2-methoxy-3-pyridyl]quinoline-5-sulfonamide;
and pharmaceutically acceptable salts thereof.

27. A process to prepare a compound according to claim 1 comprising reacting a compound of formula III with a compound of formula II in the presence of a base selected from N-ethyldiisopropylamine, pyridine, potassium phosphate or sodium hydride, to provide a compound of formula I, 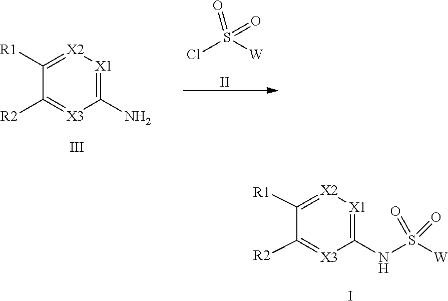
wherein R¹, R², R³, X₁, X₂, X₃ and W are as described above.

28-29. (canceled)

30. A pharmaceutical composition comprising a compound according to claim 1 and a therapeutically inert carrier.

31-35. (canceled)

36. A method for the treatment or prophylaxis of conditions resulting from direct damage to myelin sheaths (including but not limited central pontine and extra-pontine myelinolysis, carbon monoxide poisoning, nutritional deficiency, and virus-induced demyelination), demyelinating disorders (including but not limited to multiple sclerosis, acute and multiphasic disseminated encephalomyelitis, neuromyelitis optica spectrum disorders, and leukodystrophies), CNS disorders associated with myelin loss (including but not limited to Alzheimer's disease, schizophrenia, Parkinson's disease, Huntington's disease, Amyotrophic lateral sclerosis, and Ischemia due to stroke), and inflammation in the CNS for instance following encephalitis, primary angiitis, meningitis and obesity, which method comprises administering an effective amount of a compound according to claim 1 to a patient in need thereof.

37. A method for the treatment or prophylaxis of multiple sclerosis, which method comprises administering an effective amount of a compound according to claim 1 to a patient in need thereof.

38-39. (canceled)
